(19) United States
(12) Patent Application Publication
(10) Pub. No.: US 2012/0003695 A1
(43) Pub. Date: Jan. 5, 2012

(54) METABOLIC ENGINEERING OF A GALACTOSE ASSIMILATION PATHWAY IN THE GLYCOENGINEERED YEAST PICHIA PASTORIS

(76) Inventors: Robert C. Davidson, Enfield, NH (US); Piotr Bobrowicz, Hanover, NH (US); Dongxing Zha, Etna, NH (US)

(21) Appl. No.: 13/202,002

(22) PCT Filed: Feb. 24, 2010
(86) PCT No.: PCT/US2010/025163

§ 371 Date: Aug. 17, 2011

Related U.S. Application Data
(60) Provisional application No. 61/208,582, filed on Feb. 25, 2009.

(51) Int. Cl. C12P 21/00; C12N 9/64; C12N 15/81; C12N 9/16; C12N 9/24; C12N 1/19; C12N 9/72

(52) U.S. Cl. 435/69.2; 435/254.23; 435/69.1; 435/69.4; 435/69.51; 435/69.52; 435/69.6; 435/226; 435/215; 435/196; 435/200; 435/69.7; 435/471

(57) ABSTRACT

Lower eukaryotic cells such as Pichia pastoris that normally cannot use galactose as a carbon source but which have been genetically engineered according to the methods herein to use galactose as a sole source of carbon are described. The cells are genetically engineered to express several of the enzymes comprising the Leloir pathway. In particular, the cells are genetically engineered to express a galactokinase, a UDP-galactose-C4-epimerase, and a galactose-1-phosphate uridyltransferase, and optionally a galactose permease. In addition, a method is provided for improving the yield of glycoproteins that have galactose-terminated or -containing N-glycans in cells that have been genetically engineered to produce glycoproteins with N-glycans having galactose residues but which normally lack the enzymes comprising the Leloir pathway comprising transforming the cells with one or more nucleic acid molecules encoding a galactokinase, a UDP-galactose-C4-epimerase, and a galactose-1-phosphate uridyltransferase. The methods and host cells described enable the presence or lack of the ability to assimilate galactose as a selection method for making recombinant cells. The methods and host cells are shown herein to be particularly useful for making immunoglobulins and the like that have galactose-terminated or containing N-glycans.

### BACKGROUND OF THE INVENTION

(1) Field of the Invention

The present invention relates to lower eukaryotic cells, such as Pichia pastoris, that normally are unable to use galactose as a carbon source but which are rendered capable of using galactose as a sole source of carbon by genetically engineering the cells to express several of the enzymes comprising the Leloir pathway. In particular, the cells are genetically engineered to express a galactokinase, a UDP-galactose-C4-epimerase, and a galactose-1-phosphate uridyltransferase, and optionally a galactose permease. In addition, the present invention further relates to a method for improving the yield of glycoproteins that have galactose-terminated or -containing N-glycans in lower eukaryotes that have been genetically engineered to produce glycoproteins with N-glycans having galactose residues but which normally lack the enzymes comprising the Leloir pathway comprising transforming the lower eukaryote with one or more nucleic acid molecules encoding a galactokinase, a UDP-galactose-C4-epimerase, and a galactose-1-phosphate uridyltransferase.

(2) Description of Related Art

Protein-based therapeutics constitute one of the most active areas of drug discovery and are expected to be a major source of new therapeutic compounds in the next decade (Walsh, Nat. Biotechnol. 18(8): 831-3 (2000)). Therapeutic proteins, which are not glycosylated in their native state, can be expressed in hosts that lack a glycosylation machinery, such as Escherichia coli. However, most therapeutic proteins are glycoproteins, which require the post-translational addition of glycans to specific asparagine residues of the protein to ensure proper folding and subsequent stability in the human serum (Helenius and Aebi, Science 291: 2364-9 (2001)). In certain cases, the efficacy of therapeutic proteins has been improved by engineering in additional glycosylation sites. One example is the human erythropoietin, which upon the addition of two additional glycosylation sites has been demonstrated to exhibit a three-fold longer half-life in viva (Macdougall et al., J. Am. Soc. Nephrol. 10: 2392-2395 (1999)). Most glycoproteins intended for therapeutic use in humans require N-glycosylation and thus, mammalian cell lines, such as Chinese Hamster Ovary (CHO) cells, that approximate human glycoprotein processing are currently most often used for production of therapeutic glycoproteins. However, these cell lines have significant drawbacks including poor genetic tractability, long fermentation times, heterogeneous glycosylation, and ongoing viral containment issues (Birch and Racher, Adv. Drug Deliv. Rev. 58: 671-85 (2006); Kalyanpur, Mol. Biotechnol. 22: 87-98 (2002)).

Many industrial protein expression systems are based on yeast strains that can be grown to high cell density in chemically defined medium and generally do not suffer from the abovementioned limitations (Cereghino and Cregg FEMS Microbiol. Rev. 24: 45-66 (2000); Hollenberg and Gellisen, Curr. Opin. Biotechnol. 8: 554-560 (1997); Muller, Yeast 14: 1267-1283 (1998). While yeasts have been used for the production of aglycosylated therapeutic proteins, such as insulin, they have not been used for glycoprotein production because yeast produce the glycoproteins with non-human, high mannose-type N-glycans (See FIG. 1), which result in glycoproteins with a shortened in vivo half-life and which have the potential to be immunogenic in higher mammals (Tanner et al., Biochim. Biophys. Acta. 906: 81-99 (1987)).

To address these issues, the present inventors and others have focused on the re-engineering of glycosylation pathways in a variety of different yeasts and filamentous fungi to obtain human-like glycoproteins from these protein expression hosts. For example, Gerngross et al., U.S. Published Application No. 2004/0018590, the disclosure of which is hereby incorporated herein by reference, provides cells of the yeast Pichia pastoris, which have been genetically engineered to eliminate production of high mannose-type N-glycans typical of yeasts and filamentous fungi, and to provide a host cell with the glycosylation machinery to produce glycoproteins with hybrid or complex N-glycans more typical of glycoproteins produced from mammalian cells.

Gerngross et al. above discloses recombinant yeast strains that can produce recombinant glycoproteins in which a high percentage of the N-glycans thereon contain galactose residues: i.e., yeast strains that produce N-glycans having predominantly the oligosaccharide structures GalGlcNAc₂Man₃GlcNAc₂ (G1) or Gal₂GlcNAc₂Man₃GlcNAc₂ (G2) and lesser amounts of the oligosaccharide structure GlcNAc₂Man₃GlcNAc₂ (G0). Yields of 70-85% G2 have been obtained. However, it has been found some glycoproteins such as immunoglobulins and immunoadhesions are produced in these cells in which the ratio of G0:G1/G2 is reduced to about 2:1 (See for example, Li et al., Nature Biotechnol. 24: 210-215 (2006) wherein the yield of galactose-terminated N-glycans from these cells was improved by treating the glycoproteins in vitro with galactose and a soluble form of β-1,4-galactosyltransferase). In contrast, immunoglobulins produced in mammalian cells such as CHO cells have a G0:G1/G2 ratio of about 1:1. Thus, it would be desirable to provide a recombinant yeast host cell that is capable of producing recombinant glycoproteins in vivo in which the G0:G1/G2 ratio is less than 2:1.

Pichia pastoris, can use only a limited number of carbon sources for survival. Currently, these carbon sources are glycerol, glucose, methanol, and perhaps rhamnose and mannose but not galactose. It would be desirable to have Pichia pastoris strains that can use carbon source other than those listed above.

### BRIEF SUMMARY OF THE INVENTION

The present invention solves the above identified problems. The present invention provides methods and materials for generating from host cells that lack the ability to assimilate galactose as a carbon source, recombinant host cells that have the ability to use galactose as an energy source. When the recombinant host cells are further genetically engineered to produce glycoproteins that have galactose-terminated or -containing N-glycans, the host cells are capable of producing recombinant glycoproteins such as antibodies in which the G0:G1/G2 ratio is less than 2:1, or a G0:G1/G2 ratio that is about 1:1 or less, or a G0:G1/G2 ratio that is about 1:2 or less. In general, the method comprises introducing into the host cells nucleic acid molecules encoding the Leloir pathway enzymes: galactokinase, UDP-galactose-C4-epimerase, and galactose-1-phosphate uridyltransferase, and optionally a galactose permease. Thus, the methods and materials herein provide a selection system that can be used to identify host cells that have been transformed simply by growing the cells on medium containing galactose as the carbon source and provides a method for producing glycoproteins such as immunoglobulins that have a high level of galactose-terminated or -containing N-glycans.

The ability to utilize galactose as a carbon source provides flexibility and economy as to the choice of expression systems to use. For example, in systems designed for the expression of recombinant glycoproteins with terminal galactose or terminal sialylation, galactose can be added to the medium where it is taken up by the cells and used by the cells both as an energy source and to provide galactose residues for incorporation into N-glycans being synthesized on the recombinant glycoproteins. The advantage of the present invention is that by having galactose present in the medium or adding galactose during fermentation and/or induction of recombinant glycoprotein, production of the recombinant protein can result in higher levels of galactosylated or sialylated glycoprotein. Accordingly, as demonstrated with Pichia pastoris, a yeast species that normally lacks the Leloir pathway, genetically engineering Pichia pastoris in the manner disclosed herein results in recombinant Pichia pastoris cell lines that can use galactose as a sole carbon source. In addition, genetically engineering Pichia pastoris cell lines to include the Leloir pathway enzymes and the enzymes needed to render the cells capable of making glycoproteins that have galactose-terminated or -containing N-glycans results in a recombinant cell line in which the yield of galactose-terminated or -containing N-glycans is greater than when the cell line lacks the Leloir pathway enzymes. Thus, the present invention results in increased productivity in Pichia pastoris cell lines that have been genetically engineered to produce galactosylated or sialylated glycoproteins.

In particular, the present invention provides methods and materials which are useful for the production of antibodies with high levels of galactose or sialic acid in vivo. Using the methods and materials of the present invention, galactose is added to cell growth medium in order to accomplish multiple purposes including (a) selection of host cells which are able to use galactose as a sugar source; (b) providing a carbon source for the growth of the host cells; and (c) providing a source of galactose residues for incorporation into N-glycans, either as the terminal galactose residues in the N-glycans or to provide a substrate for subsequent addition of terminal sialic acid residues to the N-glycans. Thus, the present invention provides methods and materials by which levels of galactosylation can be increased through in vivo processes, rather than using less efficient and more expensive in vitro reactions in which charged galactose and a soluble galactosyl transferase enzyme are added to the medium or solution containing purified but partially galactosylated recombinant glycoproteins.

One embodiment of the present invention is the development of Pichia pastoris host cells that are capable of surviving on media in which galactose is present as the sole carbon source. Using the materials and methods of the present invention, one skilled in the art will be able to produce recombinant glycoproteins from the transformed host cells disclosed herein using galactose as the carbon source for selecting and maintaining transformed host cells. Further, by supplying the cell culture medium with galactose, the present invention can be used to increase the levels of galactosylated or sialylated glycoprotein which is produced from the cells when the host cell has been genetically engineered to produce galactosylated or sialylated N-glycans.

Therefore, a Pichia pastoris host cell is provided that has been genetically engineered to express a galactokinase activity, a UDP-galactose-4-epimerase activity, a galactose-1-phosphate uridyl transferase activity, and optionally a galactose permease activity, wherein the host cell is capable of using galactose as a sole carbon energy source.

In particular aspects, the Pichia pastoris host cell has been further genetically engineered to be capable of producing recombinant glycoproteins that have hybrid or complex N-glycans that comprise galactose residues. In particular embodiments, the UDP-galactose-4-epimerase activity is provided in a fusion protein comprising the catalytic domain of a galactosyltransferase and the catalytic domain of an UDP-galactose-4-epimerase.

In general, the host cell is capable of producing glycoproteins that have complex N-glycans in which the G0:G1/G2 ratio is less than 2:1.

In particular embodiments, the glycoproteins produced in the above cells have predominantly an N-glycan selected from the group consisting of GalGlcNAcMan₅GlcNAc₂; NANAGalGlcNAcMan₅GlcNAc₂; GalGlcNAcMan₃GlcNAc₂; NANAGalGlcNAcMan₃GlcNAc₂; GalGlcNAc₂Man₃GlcNAc₂; Gal₂GlcNAc₂Man₃GlcNAc₂; NANAGal₂GlcNAc₂Man₃GlcNAc₂; and NANA₂Gal₂GlcNAc₂Man₃GlcNAc₂.

In further embodiments, N-glycan is a galactose-terminated N-glycan selected from the group consisting of GalGlcNAcMan₅GlcNAc₂; Gal₂GlcNAc₂Man₃GlcNAc₂; and Gal₂GlcNAc₂Man₃GlcNAc₂. In other embodiments, the N-glycan is a galactose-terminated hybrid N-glycan and in further embodiments, the N-glycan is a sialylated N-glycan selected from the group consisting of: NANAGalGalNAcMan₅GlcNAc₂; NANAGal₂GlcNAc₂Man₃GlcNAc₂; and NANA₂Gal₂GlcNAc₂Man₃GlcNAc₂.

Further provided is a method of producing a recombinant glycoprotein in a Pichia pastoris host with N-glycans that have galactose residues, said method comprising; a) providing a recombinant host cell that has been genetically engineered to express (i) a glycosylation pathway that renders the host cell capable of producing recombinant glycoproteins that have hybrid or complex N-glycans that comprise galactose residues; (ii) a galactokinase activity, a UDP-galactose-4-epimerase activity, a galactose-1-phosphate uridyl transferase activity and optionally a galactose permease activity; and (iii) a recombinant glycoprotein; and b) culturing the host cells in a medium containing galactose to produce the recombinant glycoprotein that has one or more N-glycans that have galactose residues.

In further aspects of the method, the UDP-galactose-4-epimerase activity is provided in a fusion protein comprising the catalytic domain of a galactosyltransferase and the catalytic domain of an UDP-galactose-4-epimerase.

The present invention further provides a method for selecting a recombinant host cell that expresses a heterologous protein. Recombinant host cells that express one or two but not all of the Leloir pathway enzyme activities are transformed with one or more nucleic acid molecules encoding the heterologous protein and the Leloir pathway enzymes not present in the recombinant host cell. Since the transformed recombinant host cell contains a complete Leloir pathway, selection of the transformed recombinant host cell that expresses the heterologous protein from non-transformed cells can be achieved by culturing the transformed recombinant host cells in a medium in which galactose is the sole carbon source. Thus, further provided is a method for producing a recombinant host cell that expresses a heterologous protein, comprising: (a) providing a host cell that has been genetically engineered to express one or two enzymes selected from the group consisting of a galactokinase, a UDP-galactose-4-epimerase, and a galactose-1-phosphate uridyl transferase; (b) transforming the host cell with one or more nucleic acid molecules encoding the heterologous protein and the enzyme or enzymes from the group in step (a) not expressed in the host cell of step (a); and (c) culturing the host cells in a medium containing galactose as the sole carbon source to provide the recombinant Pichia pastoris host cell that expresses a heterologous protein.

In further aspects of the method, the host cell is further genetically engineered to express a galactose permease. In further still aspects, the host cell is genetically modified to produce glycoproteins that have one or more N-glycans that comprise galactose.

Further provided is a method of producing and selecting Pichia pastoris host cells capable of using galactose as a sole carbon source, the method comprising;

a) providing a Pichia pastoris host cell; b) transforming the host cell with one or more nucleic acid molecules encoding a galactokinase, a UDP-galactose-4-epimerase, a galactose-1-phosphate uridyl transferase, and optionally a galactose permease; c) culturing the transformed host cells of on a medium containing galactose as the sole carbon source; and d) selecting the host cells that can grow on the medium containing galactose as the sole carbon source.

The host cells and methods herein enable the production of compositions comprising a recombinant glycoprotein wherein the ratio of G0:G1/G2 glycoforms thereon is less than 2:1 in a pharmaceutically acceptable carrier. In particular embodiments, the recombinant glycoprotein is selected from the group consisting erythropoietin (EPO); cytokines such as interferon α, interferon β, interferon γ, and interferon ω; and granulocyte-colony stimulating factor (GCSF); GM-CSF; coagulation factors such as factor VIII, factor IX, and human protein C; antithrombin III; thrombin,; soluble IgE receptor α-chain; immunoglobulins such as IgG, IgG fragments, IgG fusions, and IgM; immunoadhesions and other Fc fusion proteins such as soluble TNF receptor-Fc fusion proteins; RAGE-Fc fusion proteins; interleukins; urokinase; chymase; and urea trypsin inhibitor; IGF-binding protein; epidermal growth factor; growth hormone-releasing factor; annexin V fusion protein; angiostatin; vascular endothelial growth factor-2; myeloid progenitor inhibitory factor-1; osteoprotegerin; α-1-antitrypsin; α-feto proteins; DNase II; kringle 3 of human plasminogen; glucocerebrosidase; TNF binding protein 1; follicle stimulating hormone; cytotoxic T lymphocyte associated antigen 4-Ig; transmembrane activator and calcium modulator and cyclophilin ligand; glucagon like protein 1; and IL-2 receptor agonist.

In further embodiments, glycoprotein is an antibody, in particular, a humanized, chimeric or human antibody. In particular embodiments, the antibody is selected from the group consisting of anti-Her2 antibody, anti-RSV (respiratory syncytial virus) antibody, anti-TNFα antibody, anti-VEGF antibody, anti-CD3 receptor antibody, anti-CD41 7E3 antibody, anti-CD25 antibody, anti-CD52 antibody, anti-CD33 antibody, anti-IgE antibody, anti-CD11a antibody, anti-EGF receptor antibody, and anti-CD20 antibody, and variants thereof. Examples of the antibodies include Muromonab-CD3, Abciximab, Rituximab, Daclizumab, Basiliximab, Palivizumab, Infliximab, Trastuzumab, Gemtuzumab ozogamicin, Alemtuzumab, Ibritumomab tiuxeten, Adalimumab, Omalizumab, Tositumomab-¹³¹I, Efalizumab, Cetuximab, Golimumab, and Bevacizumab.

In further still embodiments, the glycoprotein is an Fc fusion protein, for example etanercept.

While Pichia pastoris is proved as an example of a host cell that can be modified as disclosed herein, the methods and host cells are not limited to Pichia pastoris. The methods herein can be used to produce recombinant host cells from other lower eukaryote species that normally do not express the Leloir pathway enzymes and as such are incapable of using galactose as a carbon source. Thus, in further embodiments, the host cell is any lower eukaryote species that normally do not express the Leloir pathway enzymes.

### DEFINITIONS

Unless otherwise defined, all technical and scientific terms used herein have the same meaning as commonly understood by one of ordinary skill in the art to which this invention pertains. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999). Exemplary methods and materials are described below, although methods and materials similar or equivalent to those described herein can also be used in the practice of the present invention and will be apparent to those of skill in the art. All publications and other references mentioned herein are incorporated by reference in their entirety for the disclosure for which they are cited. In case of conflict, the present specification, including definitions, will control. The materials, methods, and examples are illustrative only and not intended to be limiting in any manner.

The following terms, unless otherwise indicated, shall be understood to have the following meanings:

As used herein, the terms “humanized,” “humanization” and “human-like” are used interchangeably, and refer to the process of engineering non-human cells, such as lower eukaryotic host cells, in a manner which results in the ability of the engineered cells to produce proteins, in particular, glycoproteins, which have glycosylation which more closely resembles mammalian glycosylation patterns than glycoproteins produced by non-engineered, wild-type non-human cell of the same species. Humanization may be performed with respect to either N-glycosylation, O-glycosylation, or both. For example, wild-type Pichia pastoris and other lower eukaryotic cells typically produce hypermannosylated proteins at N-glycosylation sites. In preferred embodiments of the present invention, “humanized” host cells of the present invention are capable of producing glycoproteins with hybrid and/or complex N-glycans; i.e., “human-like N-glycosylation.” The specific “human-like” glycans predominantly present on glycoproteins produced from the humanized host cells will depend upon the specific humanization steps that are performed.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs cotranslationally in the lumen of the ER and continues in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.” FIG. 2 shows various high mannose, hybrid, and complex N-glycans that have been produced in Pichia pastoris genetically engineered to produce mammalian-like N-glycans.

As used herein, the terms “O-glycan” and “glycoform” are used interchangeably and refer to an O-linked oligosaccharide, e.g., a glycan that is attached to a peptide chain via the hydroxyl group of either a serine or threonine residue. In fungal cells, native O-glycosylation occurs through attachment of a first mannosyl residue transferred from a dolichol monophosphate mannose (Dol-P-Man) to the protein in the endoplasmic reticulum, and additional mannosyl residues may be attached via transfer from GPD-Man in the Golgi apparatus. Higher eukaryotic cells, such as human or mammalian cells, undergo O-glycosylation through covalent attachment of N-acetyl-galactosamine (GlcNac) to the serine or threonine residue.

As used herein, the term “human-like O-glycosylation” will be understood to mean that fungal-specific phosphorylated mannose structures are reduced or eliminated, resulting in reduction or elimination of charge and beta-mannose structures, or that the predominant O-glycan species present on a glycoprotein or in a composition of glycoprotein comprises a glycan capped with a terminal residue selected from GlcNac; Gal, or NANA (or Sia). In this manner, the recombinant glycoprotein bearing predominantly human-like O-glycosylation may be recognized by a human or mammalian cell as if it were a natively produced glycoprotein, which result in improved therapeutic properties of the recombinant glycoprotein.

Abbreviations used herein are of common usage in the art, see, e.g., abbreviations of sugars, above. Other common abbreviations include “PNGase”, or “glycanase” or “glucosidase” which all refer to peptide N-glycosidase F (EC 3.2.2.18).

As used herein, the terms “antibody,” “immunoglobulin,” “immunoglobulins” and “immunoglobulin molecule” are used interchangeably. Each immunoglobulin molecule has a unique structure that allows it to bind its specific antigen, but all immunoglobulins have the same overall structure as described herein. The basic immunoglobulin structural unit is known to comprise a tetramer of subunits. Each tetramer has two identical pairs of polypeptide chains, each pair having one “light” chain (LC) (about 25 kDa) and one “heavy” chain (HC) (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Light chains (LCs) are classified as either kappa or lambda. Heavy chains (HCs) are classified as gamma, mu, alpha, delta, or epsilon, and define the antibody's isotype as IgG, IgM, IgA, IgD and IgE, respectively.

The light and heavy chains are subdivided into variable regions and constant regions (See generally, Fundamental Immunology (Paul, W., ed., 2nd ed. Raven Press, N.Y., 1989), Ch. 7. The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. The terms include naturally occurring forms, as well as fragments and derivatives. Included within the scope of the term are classes of immunoglobulins (Igs), namely, IgG, IgA, IgE, IgM, and IgD. Also included within the scope of the terms are the subtypes of IgGs, namely, IgG1, IgG2, IgG3 and IgG4. The term is used in the broadest sense and includes single monoclonal antibodies (including agonist and antagonist antibodies) as well as antibody compositions which will bind to multiple epitopes or antigens. The terms specifically cover monoclonal antibodies (including full length monoclonal antibodies), polyclonal antibodies, multispecific antibodies (for example, bispecific antibodies), and antibody fragments so long as they contain or are modified to contain at least the portion of the C_{H}2 domain of the heavy chain immunoglobulin constant region which comprises an N-linked glycosylation site of the C_{H}2 domain, or a variant thereof. Included within the terms are molecules comprising only the Fc region, such as immunoadhesins (U.S. Published Patent Application No. 20040136986), Fc fusions, and antibody-like molecules. Alternatively, these terms can refer to an antibody fragment of at least the Fab region that at least contains an N-linked glycosylation site.

The term “Fc” fragment refers to the ‘fragment crystallized’ C-terminal region of the antibody containing the C_{H}2 and C_{H}3 domains. The term “Fab” fragment refers to the ‘fragment antigen binding’ region of the antibody containing the V_{H}, C_{H}1, V_{L} and C_{L} domains.

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each mAb is directed against a single determinant on the antigen. In addition to their specificity, monoclonal antibodies are advantageous in that they can be synthesized by hybridoma culture, uncontaminated by other immunoglobulins. The term “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., (1975) Nature, 256: 495, or may be made by recombinant DNA methods (See, for example, U.S. Pat. No. 4,816,567 to Cabilly et al.).

The term “fragments” within the scope of the terms “antibody” or “immunoglobulin” include those produced by digestion with various proteases, those produced by chemical cleavage and/or chemical dissociation and those produced recombinantly, so long as the fragment remains capable of specific binding to a target molecule. Among such fragments are Fc, Fab, Fab′, Fv, F(ab′)₂, and single chain Fv (scFv) fragments. Hereinafter, the term “immunoglobulin” also includes the term “fragments” as well.

Immunoglobulins further include immunoglobulins or fragments that have been modified in sequence but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (See, for example, Intracellular Antibodies: Research and Disease Applications, (Marasco, ed., Springer-Verlag New York, Inc., 1998).

The term “catalytic antibody” refers to immunoglobulin molecules that are capable of catalyzing a biochemical reaction. Catalytic antibodies are well known in the art and have been described in U.S. Pat. Nos. 7,205,136; 4,888,281; and 5,037,750 to Schochetman et al., U.S. Pat. Nos. 5,733,757; 5,985,626; and 6,368,839 to Barbas, III et al.

The term “vector” as used herein is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”).

As used herein, the term “sequence of interest” or “gene of interest” refers to a nucleic acid sequence, typically encoding a protein, that is not normally produced in the host cell. The methods disclosed herein allow efficient expression of one or more sequences of interest or genes of interest stably integrated into a host cell genome. Non-limiting examples of sequences of interest include sequences encoding one or more polypeptides having an enzymatic activity, e.g., an enzyme which affects N-glycan synthesis in a host such as mannosyltransferases, N-acetylglucosaminyl transferases, UDP-N-acetylglucosamine transporters, galactosyltransferases, UDP-N-acetylgalactosyltransferase, sialyltransferases and fucosyltransferases.

The term “marker sequence” or “marker gene” refers to a nucleic acid sequence capable of expressing an activity that allows either positive or negative selection for the presence or absence of the sequence within a host cell. For example, the P. pastoris URA5 gene is a marker gene because its presence can be selected for by the ability of cells containing the gene to grow in the absence of uracil. Its presence can also be selected against by the inability of cells containing the gene to grow in the presence of 5-FOA. Marker sequences or genes do not necessarily need to display both positive and negative selectability. Non-limiting examples of marker sequences or genes from P. pastoris include ADE1, ARG4, HIS4 and URA3. For antibiotic resistance marker genes, kanamycin, neomycin, geneticin (or G418), paromomycin and hygromycin resistance genes are commonly used to allow for growth in the presence of these antibiotics.

The term “operatively linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “eukaryotic” refers to a nucleated cell or organism, and includes insect cells, plant cells, mammalian cells, animal cells and lower eukaryotic cells.

The term “lower eukaryotic cells” includes yeast, fungi, collar-flagellates, microsporidia, alveolates (e.g., dinoflagellates), stramenopiles (e.g, brown algae, protozoa), rhodophyta (e.g., red algae), plants (e.g., green algae, plant cells, moss) and other protists. Yeast and filamentous fungi include, but are not limited to: Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crasser.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “chimeric gene” or “chimeric nucleotide sequences” refers to a nucleotide sequence comprising a nucleotide sequence or fragment coupled to heterologous nucleotide sequences. Chimeric sequences are useful for the expression of fusion proteins. Chimeric genes or chimeric nucleotide sequences may also comprise one or more fragments or domains which are heterologous to the intended host cell, and which may have beneficial properties for the production of heterologous recombinant proteins. Generally, a chimeric nucleotide sequence comprises at least 30 contiguous nucleotides from a gene, more preferably at least 60 or 90 or more nucleotides. Chimeric nucleotide sequences which have at least one fragment or domain which is heterologous to the intended host cell, but which is homologous to the intended recombinant protein, have particular utility in the present invention. For example, a chimeric gene intended for use in an expression system using P. pastoris host cells to express recombinant human glycoproteins will preferably have at least one fragment or domain which is of human origin, such as a sequence which encodes a human protein with potential therapeutic value, while the remainder of the chimeric gene, such as regulatory sequences which will allow the host cell to process and express the chimeric gene, will preferably be of P. pastoris origin. If desired, the fragment of human origin may also be codon-optimized for expression in the host cell. (See, e.g., U.S. Pat. No. 6,884,602, hereby incorporated by reference).

The term “fusion protein” or “chimeric protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present invention have particular utility. The heterologous polypeptide included within the fusion protein of the present invention is at least 6 amino acids in length, often at least 8 amino acids in length, and usefully at least 15, 20, and 25 amino acids in length. Fusions also include larger polypeptides, or even entire proteins, such as the green fluorescent protein (“GFP”) chromophore-containing proteins having particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

The term “non-peptide analog” refers to a compound with properties that are analogous to those of a reference polypeptide. A non-peptide compound may also be termed a “peptide mimetic” or a “peptidomimetic”. See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15: 29 (1986); Veber and Freidinger, Trends Neurosci., 8: 392-396 (1985); and references sited in each of the above, which are incorporated herein by reference. Such compounds are often developed with the aid of computerized molecular modeling. Peptide mimetics that are structurally similar to useful peptides of the invention may be used to produce an equivalent effect and are therefore envisioned to be part of the invention.

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule.

As used herein, the term “molecule” means any compound, including, but not limited to, a small molecule, peptide, protein, sugar, nucleotide, nucleic acid, lipid, etc., and such a compound can be natural or synthetic.

As used herein, the term “comprise” or variations such as “comprises” or “comprising”, will be understood to imply the inclusion of a stated integer or group of integers but not the exclusion of any other integer or group of integers.

As used herein, the term “predominantly” or variations such as “the predominant” or “which is predominant” will be understood to mean the glycan species that has the highest mole percent (%) of total O-glycans or N-glycans after the glycoprotein has been treated with enzymes and released glycans analyzed by mass spectroscopy, for example, MALDI-TOF MS. In other words, the phrase “predominantly” is defined as an individual entity, such that a specific “predominant” glycoform is present in greater mole percent than any other individual entity. For example, if a composition consists of species A in 40 mole percent, species Ban 35 mole percent and species C in 25 mole percent, the composition comprises predominantly species A.

### BRIEF DESCRIPTION OF THE DRAWINGS

FIG. 1 illustrates the N-glycosylation pathways in humans and P. pastoris. Early events in the ER are highly conserved, including removal of three glucose residues by glucosidases I and II and trimming of a single specific α-1,2-linked mannose residue by the ER mannosidase leading to the same core structure, Man₈GlcNAc₂ (Man8B). However, processing events diverge in the Golgi. Mns, α-1,2-mannosidase; MnsII, mannosidase H; GnT I, α-1,2-N-acetylglucosaminyltransferase I; GnT II, α-1,2-N-acetylglucosaminyltransferase II; MnT, mannosyltransferase. The two core GlcNAc residues, though present in all cases, were omitted in the nomenclature.

FIG. 2 illustrates the key intermediate steps in N-glycosylation as well as a shorthand nomenclature referring to the genetically engineered Pichia pastoris strains producing the respective glycan structures (GS).

FIG. 3 illustrates MALDI-TOF Mass Spectroscopy (MS) analysis of N-glycosidase F released N-glycans. K3 (the kringle 3 domain of human Plasminogen) was produced in P. pastoris strains GS115-derived wild-type control (Invitrogen, Carlsbad, Calif.), YSH44, YSH71, RDP52, and RDP80 and purified from culture supernatants by Ni-affinity chromatography. N-glycans were released by N-glycosidase F treatment and subjected to MALDI-TOF MS analysis (positive mode, except for FIG. 3G which was negative mode) appearing as sodium or potassium adducts. The two core GlcNAc residues, though present, were omitted in the nomenclature. GN, GlcNAc; M, mannose.

FIG. 3A: N-glycans produced in GS115-derived wild-type control strain;

FIG. 3B: N-glycans produced on K3 in strain YSH44;

FIG. 3C: N-glycans produced on K3 in strain YSH71 (YSH44 expressing hGalTI),

FIG. (3D) N-glycans produced on K3 in strain RDP52 (YSH44 expressing hGalTI and SpGALE),

FIG. (3E) N-glycans produced on K3 in strain RDP80 (YSH44 expressing hGaITI, SpGALE, and DmUGT);

FIG. (3F) glycans from RDP80 after α-galactosidase treatment in vitro; and

FIG. (3G) glycans from RDP80 in negative mode after treatment with α-2,6-(N)-sialyltransferase.

FIG. 4 illustrates the Leloir galactose utilization pathway. Extracellular galactose is imported via a galactose permease. The galactose is converted into glucose-6-phosphate by the action of the enzymes galactokinase, galactose-1-phosphate uridyltransferase, and UDP-galactose C4-epimerase. Protein names from S. cerevisiae are in parentheses.

FIG. 5 shows the construction of P. pastoris glycoengineered strain YGLY578-1. The P. pastoris genes OCH1, MNN4, PNO1, MNN4L1, and BMT2 encoding Golgi glycosyltransferases were knocked out followed by knock-in of 12 heterologous genes, including the expression cassette for secreted hK3. YGLY578-1 is capable of producing glycoproteins that have Gal₂GlcNAc₂Man₃GlcNAc₂ N-glycans. CS, counterselect.

FIG. 6 shows a feature diagram of plasmid pRCD977b. This plasmid is an arg1::HIS1 knock out plasmid that integrates into and deletes the P. pastoris ARG1 gene while using the PpHIS1 gene as a selectable marker and contains expression cassettes encoding the full-length D. melanogaster Golgi UDP-galactose transporter (DmUGT), full-length S. cerevisiae galactokinase (ScGAL1), full-length S. cerevisiae galactose-1-phosphate uridyl transferase (ScGAL7), and S. cerevisiae galactose permease (ScGAL2) under the control of the PpOCH₁, PpGAPDH, PpPMA1, and PpTEF promoters, respectively. TT refers to transcription termination sequence.

FIG. 7 shows that glycoengineered P. pastoris strains expressing S. cerevisiae GAL1, GAL2, and GAL7 genes can grow on galactose as a sole carbon source whereas the parent strain cannot. Glycoengineered strain YGLY578-1, which expresses the SeGAL10 and hGalTIβ, was transformed with the plasmid pRCD977b, which contains expression cassettes encoding ScGAL1, ScGAL7, and ScGAL2. Strains were cultivated on defined medium containing yeast nitrogen base, biotin, and either 3% galactose or 2% glucose as a carbon source, or neither.

FIG. 8 shows the construction of P. pastoris glycoengineered strain YGLY317-36. P. pastoris strain YGLY16-3 was generated by knock-out of five yeast glycosyltransferases. Subsequent knock-in of eight heterologous genes, yielded RDP696-2, a strain capable of transferring the human N-glycan Gal₂GlcNAc₂Man₃GlcNAc₂ to secreted proteins. Selection of robust clones via CSTR cultivation and introduction of a plasmid expressing secreted human Fc yielded strain YGLY317-36. CS, counterselect.

FIG. 9 shows a feature diagram of plasmid pGLY954. This plasmid is a KINKO plasmid that integrates into the P. pastoris TRP1 locus without deleting the gene. The plasmid contains expression cassettes encoding the full-length S. cerevisiae galactokinase (ScGAL1) and the full-length S. cerevisiae galactose-1-phosphate uridyl transferase (ScGAL7) under the control of the PpHHT1 and PpPMA1 promoters, respectively. The plasmid also contains an expression cassette encoding a secretory pathway targeted fusion protein (CO hGalTI) comprising the ScMnt1 (ScKre2) leader peptide (33) fused to the N-terminus of the human Galactosyl Transferase I catalytic domain under the control of the PpGAPDH promoter. TT refers to transcription termination sequence.

FIG. 10 shows a MALDI-TOF MS analysis of the N-glycans on a human Fc fragment produced in strains PBP317-36 and RDP783 either induced in BMMY medium alone or in medium containing glucose or galactose. Strains were inoculated from a saturated seed culture to about one OD, cultivated in 800 mL of BMGY for 72 hours, then split and 100 mL aliquots of culture broths were centrifuged and induced for 24 hours in 25 mL of BMMY, 25 mL of BMMY+0.5% glucose, or 25 mL of BMMY+0.5% galactose. Protein A purified protein was subjected to Protein N-glycosidase F digestion and the released N-glycans analyzed by MALDI-TOF MS. Figures A-C, N-glycans on the human Fc produced in strain PBP317-36; Figures D-E, N-glycans on the human Fc produced in strain RDP783.

FIG. 11 shows the construction of P. pastoris glycoengineered strain YDX477. P. pastoris strain YGLY16-3 (Δoch1, Δpno1, Δbmt2, Δmnn4a, Δmnn4b) was generated by knock-out of five yeast glycosyltransferases. Subsequent knock-in of eight heterologous genes, yielded RDP697-1, a strain capable of transferring the human N-glycan Gal₂GlcNAc₂Man₃GlcNAc₂ to secreted proteins. Introduction of a plasmid expressing a secreted antibody and a plasmid expressing a secreted form of Trichoderma reesei MNS1 yielded strain YDX477. CS, counterselect

FIG. 12 shows a feature diagram of plasmid pGLY1418. This plasmid is a KINKO plasmid that integrates into the P. pastoris TRP1 locus without deleting the gene. The plasmid contains expression cassettes encoding the full-length ScGAL1 and ScGAL7 under the control of the PpHHT1 and PpPMA1 promoters, respectively. The plasmid also contains an expression cassette encoding a secretory pathway targeted fusion protein (hGalTI) comprising the ScMnt1 (ScKre2) leader peptide fused to the N-terminus of the human Galactosyl Transferase I catalytic domain under the control of the PpGAPDH promoter. TT refers to transcription termination sequence.

FIG. 13A-F shows a MALDI-TOF MS analysis of N-glycans on an anti-Her2 antibody produced in strains YDX477 and RDP968-1 either induced in BMMY medium alone or in medium containing galactose. Strains were cultivated in 150 mL of BMGY for 72 hours, then split and 50 mL aliquots of culture broths were centrifuged and induced for 24 hours in 25 mL of BMMY, 25 mL of BMMY+0.1% galactose, or 25 mL of BMMY+0.5% galactose. Protein A purified protein was subjected to Protein N-glycosidase F digestion and the released N-glycans analyzed by MALDI-TOF MS. FIGS. 13A-C, N-glycans on the antibody produced in strain YDX477; FIGS. 13D-F, N-glycans on the antibody produced in strain RDP968-1.

FIG. 14 shows a feature diagram of plasmid pAS24. This plasmid is a P. pastoris bmt2 knock-out plasmid that contains the PpURA3 selectable marker and contains an expression cassette encoding the full length Mouse Golgi UDP-GlcNAc Transporter (MmSLC35A3) under control of the PpOCH1 promoter. TT refers to transcription termination sequence.

FIG. 15 shows a feature diagram of plasmid pRCD742b. This plasmid is a KINKO plasmid that contains the PpURA5 selectable marker as well as expression cassette encoding a secretory pathway targeted fusion protein (FB8 MannI) comprising a ScSec12 leader peptide fused to the N-terminus of a mouse Mannosidase I catalytic domain under control of the PpGAPDH promoter, an expression cassette encoding a secretory pathway targeted fusion protein (CONA10) comprising a PpSec12 leader peptide fused to the N-terminus of a human GlcNAc Transferase I (GnT I) catalytic domain under control of the PpPMA1 promoter, and a full length gene encoding the Mouse Golgi UDP-GlcNAc transporter (MmSLC35A3) under control of the PpSEC4 promoter. TT refers to transcription termination sequence.

FIG. 16 shows a feature diagram of Plasmid pDMG47. The plasmid comprises an expression cassette encoding a secretory pathway targeted fusion protein (KD53) comprising the ScMnn2 leader peptide fused to the N-terminus of the catalytic domain of the Drosophila melanogaster Mannosidase II under control of the PpGAPDH promoter. The plasmid also contains an expression cassette encoding a secretory pathway targeted fusion protein (TC54) comprising the ScMnn2 leader peptide fused to the N-terminus of the catalytic domain of the rat GlcNAc Transferase II (GnT II) under control of the PpPMA1 promoter. TT refers to transcription termination sequence.

FIG. 17 shows a feature diagram of plasmid pRCD823b. This plasmid is a KINKO plasmid that integrates into the P. pastoris HIS4 locus without deleting the gene, and contains the PpURA5 selectable marker. The plasmid comprises an expression cassette encoding a secretory pathway targeted fusion protein (TA54) comprising the ScMnn2 leader peptide fused to the N-terminus of the rat GlcNAc Transferase II (GnT II) catalytic domain under the control of the PpGAPDH promoter and expression cassettes encoding the full-length D. melanogaster Golgi UDP-galactose transporter (DmUGT) and the S. cerevisiae UDP-galactose C4-epimerase (ScGAL10) under the control of the PpOCH1 and PpPMA1 promoters respectively. TT refers to transcription termination sequence.

FIG. 18 shows a feature diagram of plasmid pGLY893a. This plasmid is a P. pastoris his1 knock-out plasmid that contains the PpARG4 selectable marker. The plasmid contains an expression cassette encoding a secretory pathway targeted fusion protein (KD10) comprising the PpSEC12 leader peptide fused to the N-terminus of the Drosophila melanogaster Mannosidase II catalytic domain under control of the PpPMA1 promoter, an expression cassette encoding a secretory pathway targeted fusion protein (TA33) comprising the ScMnt1 (ScKre2) leader peptide fused to the N-terminus of the rat GlcNAc Transferase II (GnT II) catalytic domain under the control of the PpTEF promoter, and an expression cassette encoding a secretory pathway targeted fusion protein comprising the ScMnn2 leader peptide used to the N-terminus of the human Galactosyl Transferase I catalytic domain under the control of the PpGAPDH promoter. TT refers to transcription termination sequence.

FIG. 19 shows a feature diagram of plasmid pRCD742a. This plasmid is a KINKO plasmid that integrates into the P. pastoris ADE1 locus without deleting the gene, and contains the PpURA5 selectable marker. The plasmid contains an expression cassette encoding a secretory pathway targeted fusion protein (FB8 MannI) comprising the ScSEC12 leader peptide fused to the N-terminus of the mouse Mannosidase I catalytic domain under the control of the PpGAPDH promoter, an expression cassette encoding a secretory pathway targeted fusion protein (CONA10) comprising the PpSEC12 leader peptide fused to the N-terminus of the human GlcNAc Transferase I (GnT I) catalytic domain under the control of the PpPMA1 promoter, and an expression cassette encoding the full length mouse Golgi UDP-GlcNAc transporter (MmSLC35A3) under the control of the PpSEC4 promoter. TT refers to transcription termination sequence.

FIG. 20 shows a feature diagram of plasmid pRCD1006. This plasmid is a P. pastoris his1 knock-out plasmid that contains the PpURA5 gene as a selectable marker. The plasmid contains an expression cassette encoding a secretory pathway targeted fusion protein (XB33) comprising the ScMnt1 (ScKre2) leader peptide fused to the N-terminus of the human Galactosyl Transferase I catalytic domain under the control of the PpGAPDH promoter and expression cassettes encoding the full-length D. melanogaster Golgi UDP-galactose transporter (DmUGT) and the S. pombe UDP-galactose C4-epimerase (SpGALE) under the control of the PpOCH1 and PpPMA1 promoters, respectively. TT refers to transcription termination sequence.

FIG. 21 shows a feature diagram of plasmid pGLY167b. The plasmid is a P. pastoris arg1 knock-out plasmid that contains the PpURA3 selectable marker and contains an expression cassette encoding a secretory pathway targeted fusion protein (CO-KD53) comprising the ScMNN2 leader peptide fused to the N-terminus of the Drosophila melanogaster Mannosidase II catalytic domain under the control of the PpGAPDH promoter and an expression cassette encoding a secretory pathway targeted fusion protein (CO-TC54) comprising the ScMnn2 leader peptide fused to the N-terminus of the rat GlcNAc Transferase II (GnT II) catalytic domain under the control of the PpPMA1 promoter. TT refers to transcription termination sequence.

FIG. 22 shows a feature diagram of plasmid pBK138. The plasmid is a roll-in plasmid that integrates into the P. pastoris AOX1 promoter while duplicating the promoter. The plasmid contains an expression cassette encoding a fusion protein comprising the S. cerevisiae Alpha Mating Factor pre-signal sequence fused to the N-terminus of the human Fc antibody fragment (C-terminal 233-aa of human IgG1 H chain). TT refers to transcription termination sequence.

FIG. 23 shows a feature diagram of plasmid pGLY510. The plasmid is a roll-in plasmid that integrates into the P. pastoris TRP2 gene while duplicating the gene and contains an AOX1 promoter-ScCYC1 terminator expression cassette as well as the PpARG1 selectable marker. TT refers to transcription termination sequence.

FIG. 24 shows a feature diagram of plasmid pDX459-1. The plasmid is a roll-in plasmid that targets and integrates into the P. pastoris AOX2 promoter and contains the Zeo^{R} while duplicating the promoter. The plasmid contains separate expression cassettes encoding an anti-HER2 antibody Heavy chain and an anti-HER2 antibody Light chain, each fused at the N-terminus to the Aspergillus niger alpha-amylase signal sequence and under the control of the P. pastoris AOX1 promoter. TT refers to transcription termination sequence.

FIG. 25 shows a feature diagram of plasmid pGLY1138. This plasmid is a roll-in plasmid that integrates into the P. pastoris ADE1 locus while duplicating the gene and contains a ScARR3 selectable marker gene cassette that confers arsenite resistance as well as an expression cassette encoding a secreted Trichoderma reesei MNS1 comprising the MNS1 catalytic domain fused at its N-terminus to the S. cerevisiae alpha factor pre signal sequence under the control of the PpAOX1 promoter. TT refers to transcription termination sequence.

### DETAILED DESCRIPTION OF THE INVENTION

Yeast have been successfully used for the production of recombinant proteins, both intracellular and secreted (See for example, Cereghino Cregg FEMS Microbiology Reviews 24(1): 45-66 (2000); Harkki, et al. Bio-Technology 7(6): 596 (1989); Berka, et al. Abstr. Papers Amer. Chem. Soc. 203: 121-BIOT (1992); Svetina, et al. J. Biotechnol. 76(2-3): 245-251 (2000)). Various yeasts, such as K lactis, Pichia pastoris, Pichia methanolica, and Hansenula polymorpha, have played particularly important roles as eukaryotic expression systems for producing recombinant proteins because they are able to grow to high cell densities and secrete large quantities of recombinant protein. However, glycoproteins expressed in any of these eukaryotic microorganisms differ substantially in N-glycan structure from those produced in mammals. This difference in glycosylation has prevented the use of yeast or filamentous fungi as hosts for the production of many therapeutic glycoproteins.

To enable the use of yeast to produce therapeutic glycoproteins, yeast have been genetically engineered to produce glycoproteins having hybrid or complex N-glycans. Recombinant yeast capable of producing compositions comprising particular hybrid or complex N-glycans have been disclosed in for example, U.S. Pat. No. 7,029,872 and U.S. Pat. No. 7,449,308. In addition, Hamilton et al., Science 313:1441-1443 (2006) and U.S. Published application No. 2006/0286637 reported the humanization of the glycosylation pathway in the yeast Pichia pastoris and the secretion of a recombinant human glycoprotein with complex N-glycosylation with terminal sialic acid. A precursor N-glycan for terminal sialic acid having the oligosaccharide structure Gal₂GlcNAc₂Man₃GlcNAc₂ (G2) is a structure that has also been found as the predominant N-glycan on several proteins isolated from human serum including, follicle stimulating hormone (FSH), asialotransferrin and, most notably, in differing amounts on human immunoglobulins (antibodies). Davidson et al. in U.S. Published Application No. 2006/0040353 teaches an efficient process for obtaining galactosylated glycoproteins using yeast cells that have been genetically engineered to produce galactose terminated N-glycans. These host cells are capable of producing glycoprotein compositions having various mixtures of G2 or G1 (GalGlcNAc₂Man₃GlcNAc₂) oligosaccharide structures with varying amount of G0 oligosaccharide structures. G0 is GlcNAc₂Man₃GlcNAc₂, which is a substrate for galactosyltransferase. However, for certain glycoproteins, in particular antibodies and Fc fragments, it has been found that the efficiency of the galactose transfer process is less than optimal. In the case of antibodies and Fc fragments, it is believed that the location and accessibility of the glycosylation sites on the antibody or Fc fragment during intracellular processing inhibit efficient galactose transfer onto the N-glycan of the antibody or Fc fragment. Thus, the amount of oligosaccharide structures containing galactose is less that what has been observed for other glycoproteins such as the Kringle 3 protein. To overcome this problem, the present invention provides a means for increasing the amount of galactose transfer onto the N-glycan of the antibody or Fc fragment, thus increasing the amount of G1 and G2 containing antibodies or Fc fragments over G0 containing antibodies or Fc fragments.

Pichia pastoris, can use only a limited number of carbon sources for survival. Currently, these carbon sources are known to be glycerol, glucose, methanol, and perhaps rhamnose and mannose but not galactose. In many commercial production processes using Pichia pastoris, expression of recombinant proteins is under control of the AOX promoter, which is active in the presence of methanol but is repressed in the presence of glycerol. Thus, Pichia pastoris is usually grown in a medium containing glycerol or glycerol/methanol until the concentration of cells reaches a desired level at which time expression of the recombinant protein is by replacing the medium with medium containing only methanol as the carbon source. However, the cells are in a low energy state because methanol contains only one carbon which makes it a poor carbon source. Thus, it would be desirable to have a production process in which the Pichia pastoris could use a higher energy source such as galactose. The present invention solves this problem as well by providing genetically engineered Pichia pastoris that are able to use galactose as a sole carbon source.

Thus, the present invention has solved both of the above identified problems. The present invention provides recombinant lower eukaryote cells, in particular yeast and fungal cells, that have been glycoengineered to produce glycoproteins such as antibodies or Fc fragments in which the level of terminal galactose on the N-glycans thereon is increased compared to cells that have not been genetically engineered as taught herein. The genetically engineered host cells can be used in methods for making glycoproteins having N-glycans containing galactose wherein the amount of galactose in the N-glycan is higher than what would be obtainable in host cells that have not been genetically engineered as taught herein. The present invention also provides genetically engineered host cells wherein host cells that normally are incapable of using galactose as a sole carbon source have been genetically engineered as taught herein to be capable of using galactose as a sole carbon source. The methods herein for rendering host cells capable of using galactose as a sole carbon source used Pichia pastoris as a model. The methods herein can be used to render other yeast or fungal species that normally cannot use galactose as a carbon source capable of using galactose as a carbon source.

To solve both problems, genetically engineered host cells, which have been genetically engineered be capable of producing galactose-terminated N-glycans, are further genetically engineered to express the Leloir pathway enzymes: a galactokinase (EC 2.7.1.6), a UDP-galactose-4-epimerase (EC 5.1.3.2), and a galactose-1-phosphate uridyl transferase (EC 2.7.7.12). Optionally, the host cells can further express a galactose permease. This enables the host cells to use galactose as a carbon source and to produce a pool of UDP-galactose, which in turn serves as a substrate for galactose transferase involved in the synthesis of N-glycans that include galactose residues. Thus, the host cells and methods herein enable the production of glycoprotein compositions, in particular, antibody and Fc fragment compositions, wherein the proportion of galactose-terminated N-glycans is higher than which is obtainable in glycoengineered lower eukaryote cells. The recombinant host cells can produce recombinant glycoproteins such as antibodies and Fc fusion proteins in which the G0:G1/G2 ratio is less than 2:1, or a G0:G1/G2 ratio that is about 1:1 or less, or a G0:G1/G2 ratio that is about 1:2 or less.

The host cells and methods described herein are particularly useful for producing antibodies and Fc fragment containing fusion proteins that have N-glycans that are terminated with galactose residues. The N-glycan at Asn-297 of the heavy chain of antibodies or antibody fragments is important to the structure and function of an antibody. These functions include Fc gamma receptor binding, ability to activate complement, ability to activate cytotoxic T cells (ADCC), and serum stability. However, current antibody production in yeast or mammalian cells generally suffers from a lack of control over N-glycosylation, particularly that which occurs at Asn-297 of the constant or Fc region of the heavy chain, and particularly in the ability to control the level of terminal galactose on the N-glycans. In general, it has been found that while yeast cells that have been genetically engineered to produce glycoproteins that include galactose residues in the N-glycan can produce many glycoproteins with N-glycans that contain galactose efficiently, the ability of the cells to produce antibodies with N-glycans that contain galactose is not as efficient. As shown herein, the host cells and methods herein provide host cells that can produce antibodies in which a higher level of the antibodies have N-glycans containing galactose than in cells that are not genetically engineered as described herein.

While terminal galactose levels on N-glycans that do not have terminal galactose residues can be increased in vitro in a reaction that uses a soluble galactosyltransferase to add a charged galactose residue to the termini of the N-glycans, this in vitro process is expensive, cumbersome, and not easily scalable for production quantities of the glycoprotein. Thus, the host cells disclosed herein and which are capable of producing secreted glycoproteins, including antibodies or antibody fragments, with N-glycans having increased levels of terminal galactose in vivo provide a more desirable means for producing antibody compositions with increased levels of galactose-containing N-glycans. Thus, the present invention is a significant advancement in antibody production and provides for the first time, the ability to control particular antibody characteristics, e.g., level of galactose in the N-glycans, and in particular, the ability to produce recombinant glycoproteins with improved functional characteristics.

In addition, when the methods herein are used with host cells that have been genetically engineered to make glycoproteins that have sialylated N-glycans. Galactose terminated N-glycans are a substrate for sialyltransferase. Therefore, the amount of sialylated N-glycans is, in part, a function of the amount of galactose-terminated N-glycans available for sialylation. The host cells and methods herein provide a means for increasing the amount of galactose-terminated or -containing N-glycans, which when produced in a host cell that has been genetically engineered to make sialylated N-glycans, results in a host cell that makes an increased amount of sialylated N-glycans compared to the host cell not genetically engineered as taught herein.

While the present invention is useful for producing glycoproteins comprising galactose-terminated or -containing N-glycans, the present invention is also useful as a selection method for selecting a recombinant host cell that expresses a heterologous protein of any type, glycoprotein or not. Recombinant host cells that express one or two but not all of the Leloir pathway enzyme activities are transformed with one or more nucleic acid molecules encoding the heterologous protein and the Leloir pathway enzymes not present in the recombinant host cell. Since the transformed recombinant host cell contains a complete Leloir pathway, selection of the transformed recombinant host cell that expresses the heterologous protein from non-transformed cells can be achieved by culturing the transformed recombinant host cells in a medium in which galactose is the sole carbon source. Thus, provided is a method for producing a recombinant host cell that expresses a heterologous protein, comprising the following steps. Providing a host cell that has been genetically engineered to express one or two Leloir pathway enzymes selected from the group consisting of galactokinase, UDP-galactose-4-epimerase, and galactose-1-phosphate uridyl transferase. In some embodiments, a host cell is capable of making glycoproteins that have human-like N-glycans, and in other embodiments, the host cell does not make glycoproteins that have human-like N-glycans because the heterologous protein that is to be expressed in the host cell does not have N-glycans. The host cell is transformed with one or more nucleic acid molecules encoding the heterologous protein and the Leloir pathway enzyme or enzymes not expressed in the provided recombinant host cell. The transformed host cell is cultured in a medium containing galactose as the sole carbon source to provide the recombinant host cell that expresses the heterologous protein. Optionally, the host cell can further include a nucleic acid molecule encoding a galactose permease. In particular embodiments, the host cells are genetically engineered to control O-glycosylation or grown under conditions that control O-glycosylation or both. In further embodiments, the host cells further have been modified to reduce phosphomannosyltransferase and/or beta-mannosyltransferase activity.

### Genetically Engineering Glycosylation Pathways in Lower Eukaryotes

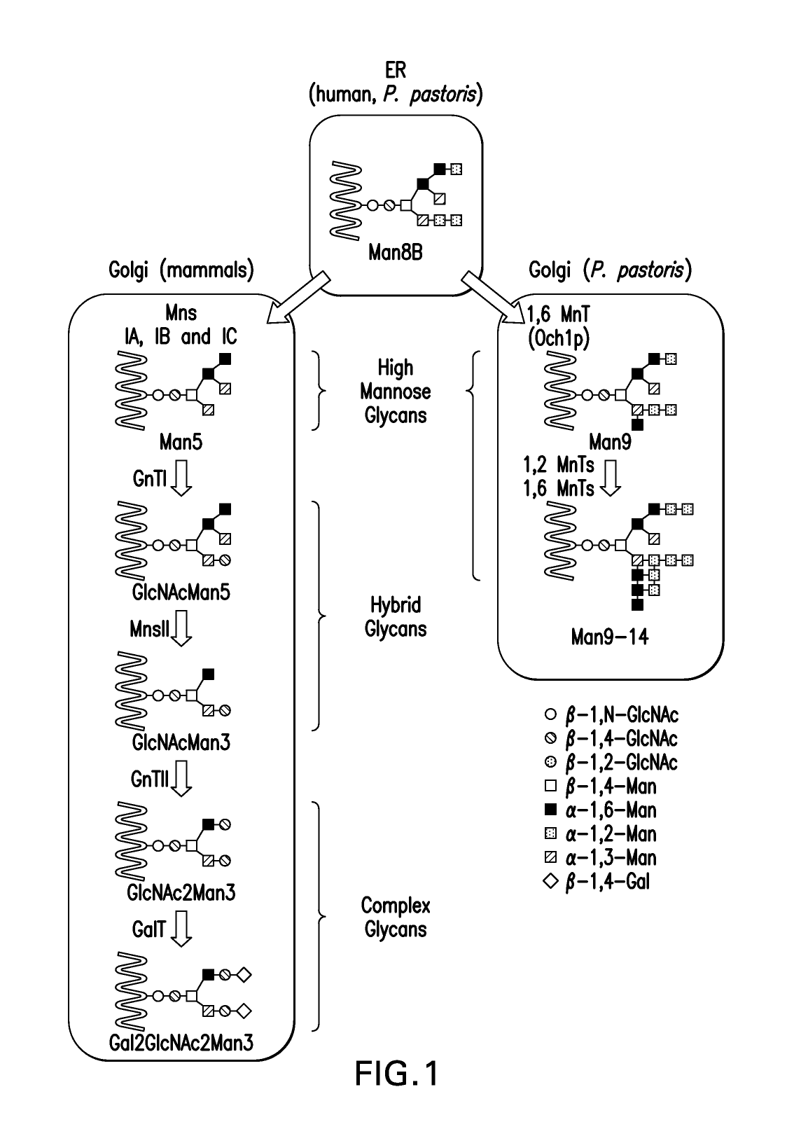

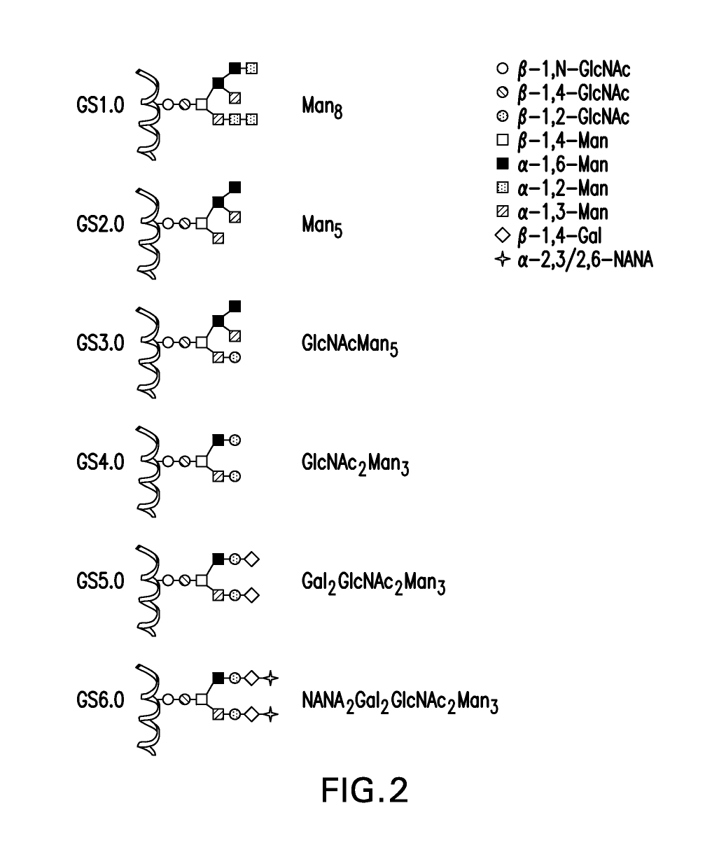

N-glycosylation in most eukaryotes begins in the endoplasmic reticulum (ER) with the transfer of a lipid-linked Glc₃Man₉GlcNAc₂ oligosaccharide structure onto specific Asn residues of a nascent polypeptide (Lehle and Tanner, Biochim. Biophys. Acta 399: 364-74 (1975); Kornfeld and Kornfeld, Annu. Rev. Biochem 54: 631-64 (1985); Burda and Aebi, Biochim. Biophys. Acta-General Subjects 1426: 239-257 (1999)). Trimming of all three glucose moieties and a single specific mannose sugar from the N-linked oligosaccharide results in Man₈GlcNAc₂ (See FIG. 1), which allows translocation of the glycoprotein to the Golgi apparatus where further oligosaccharide processing occurs (Herscovics, Biochim. Biophys. Acta 1426: 275-285 (1999); Moremen et al., Glycobiology 4: 113-125 (1994)). It is in the Golgi apparatus that mammalian N-glycan processing diverges from yeast and many other eukaryotes, including plants and insects. Mammals process N-glycans in a specific sequence of reactions involving the removal of three terminal α-1,2-mannose sugars from the oligosaccharide before adding GlcNAc to form the hybrid intermediate N-glycan GlcNAcMan₅GlcNAc₂ (Schachter, Glycoconj. J. 17: 465-483 (2000)) (See FIG. 1). This hybrid structure is the substrate for mannosidase II, which removes the terminal α-1,3- and α-1,6-mannose sugars on the oligosaccharide to yield the N-glycan GlcNAcMan₃GlcNAc₂ (Moremen, Biochim. Biophys. Acta 1573(3): 225-235 (1994)). Finally, as shown in FIG. 1, complex N-glycans are generated through the addition of at least one more GlcNAc residue followed by addition of galactose and sialic acid residues (Schachter, (2000), above), although sialic acid is often absent on certain human proteins, including IgGs (Keusch et al., Clin. Chim. Acta 252: 147-158 (1996); Creus et al., Clin. Endocrinol. (Oxf) 44: 181-189 (1996)).

In Saccharomyces cerevisiae, N-glycan processing involves the addition of mannose sugars to the oligosaccharide as it passes throughout the entire Golgi apparatus, sometimes leading to hypermannosylated glycans with over 100 mannose residues (Trimble and Verostek, Trends Glycosci. Glycotechnol. 7: 1-30 (1995); Dean, Biochim. Biophys. Acta-General Subjects 1426: 309-322 (1999)) (See FIG. 1). Following the addition of the first α-1,6-mannose to Man₈GlcNAc₂ by α-1,6-mannosyltransferase (Och1p), additional mannosyltransferases extend the Man₉GlcNAc₂ glycan with α-1,2-, α-1,6-, and terminal α-1,3-linked mannose as well as mannosylphosphate. Pichia pastoris is a methylotrophic yeast frequently used for the expression of heterologous proteins, which has glycosylation machinery similar to that in S. cerevisiae, (Bretthauer and Castellino, Biotechnol. Appl. Biochem. 30: 193-200 (1999); Cereghino and Cregg, Ferns Microbiol. Rev.24: 45-66 (2000); Verostek and Trimble, Glycobiol. 5: 671-681 (1995)). However, consistent with the complexity of N-glycosylation, glycosylation in P. pastoris differs from that in S. cerevisiae in that it lacks the ability to add terminal α-1,3-linked mannose, but instead adds other mannose residues including phosphomannose and β-linked mannose (Miura et al., Gene 324: 129-137 (2004); Blanchard et al., Glycoconj. J. 24: 33-47 (2007); Mille et al., J. Biol. Chem. 283: 9724-9736 (2008)).

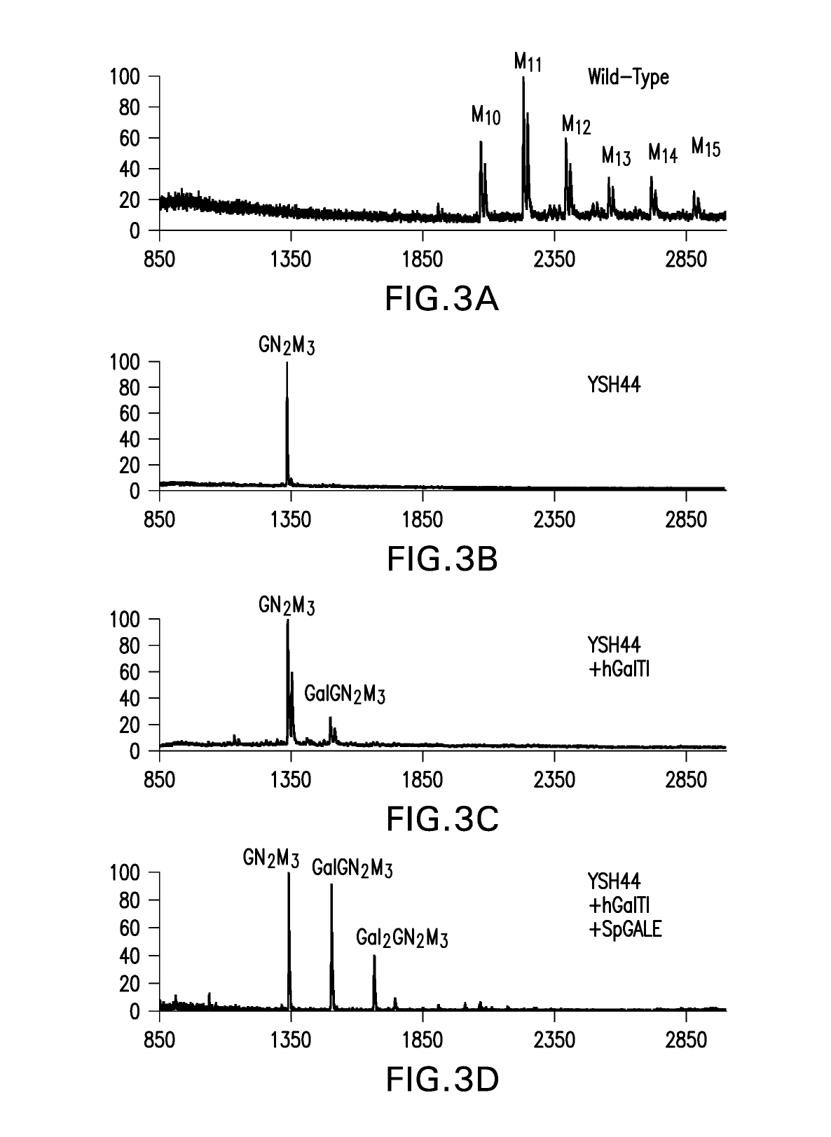

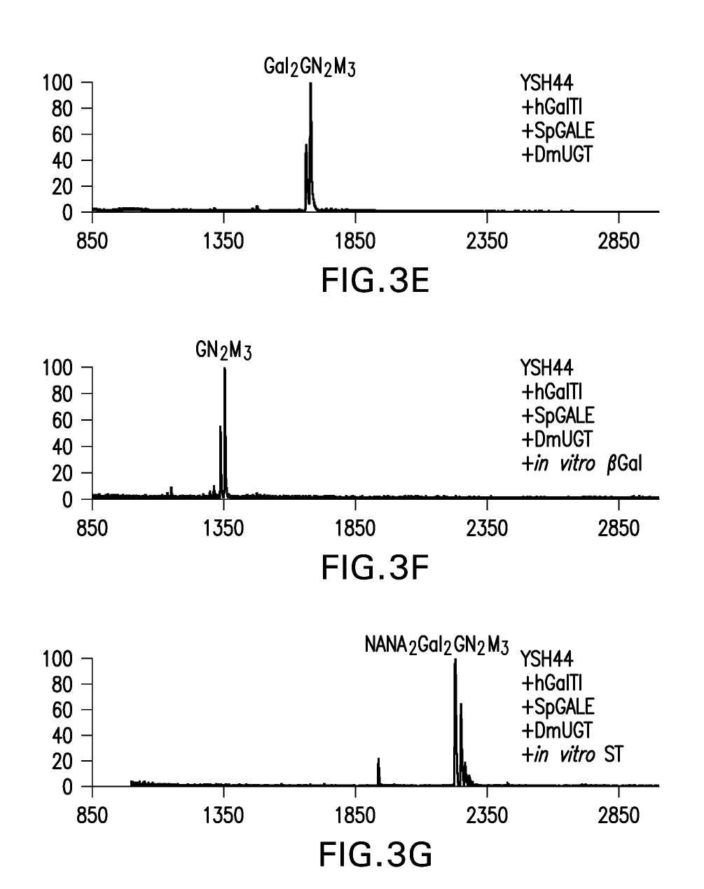

In previous work, we demonstrated that an och1 mutant of P. pastoris lacked the ability to initiate yeast-type outer chain formation and, therefore, was not able to hypermannosylate N-glycans (Choi et al., Proc. Natl. Acad. Sci. USA 100: 5022-5027 (2003)). Furthermore, we identified a novel gene family encoding Golgi-residing enzymes responsible for β-mannose transfer and demonstrated that deletion of various members of this family reduces or eliminates immunogenic β-mannose transfer (See U.S. Published Application No. US2006/0211085). Subsequent introduction of five separate glycosylation enzymes yielded a strain that produced complex human N-glycans on a secreted model protein (See FIG. 3A vs. FIG. 3B) (Choi et al., Proc. Natl. Acad. Sci. USA 100: 5022-5027 (2003); Hamilton et al. Science 310: 1244-1246 (2003); U.S. Pat. No. 7,029,872; U.S. Published Application No. 2004/0018590; U.S. Published Application No. 2004/023004; U.S. Published Application No. 2005/0208617; U.S. Published Application Number 2004/0171826; U.S. Published Application No. 2005/0208617; U.S. Published Application No. 2005/0170452; and U.S. Published Application No. 2006/0040353. More recently, the construction of recombinant yeast strains capable of producing fully sialylated N-glycans on a secreted protein produced by the yeast strain have been described in U.S. Published Application Nos. 2005/0260729 and 2006/0286637 and Hamilton et al., Science 313: 1441-1443 (2006).

The maturation of complex N-glycans involves the addition of galactose to terminal GlcNAc moieties, a reaction that can be catalyzed by several galactosyltransferases (GalTs). In humans, there are seven isoforms of GalTs (I-VII), at least four of which have been shown to transfer galactose to terminal GlcNAc in the presence of UDP-galactose in vitro (Guo, et al., Glycobiol. 11: 813-820 (2001)). The first enzyme identified, known as GalTI, is generally regarded as the primary enzyme acting on N-glycans, which is supported by in vitro experiments, mouse knock-out studies, and tissue distribution analysis (Berger and Rohrer, Biochimie 85: 261-74 (2003); Furukawa and Sato, Biochim. Biophys. Acta 1473: 54-66 (1999)). As shown herein, expression of human GalTI, when properly localized in the Golgi apparatus of the host cell, can transfer galactose onto complex N-glycans in a glycoengineered yeast strain capable of generating the terminal GlcNAc-containing precursor. Moreover, expression of a UDP-galactose 4-epimerase to generate a pool of UDP-galactose and a UDP-galactose transporter to move the substrate into the Golgi yields nearly quantitative transfer of β-1,4-galactose onto N-glycans in a strain capable of generating the terminal GlcNAc precursor. Iterative screening of a localization sub-library yielded improved generation of complex N-glycan structures over hybrid N-glycan structures presumably via separation of and reduced competition amongst GlcNAc and galactosyltransferases for intermediate N-glycan substrates.

Previously, human GalTI was shown to be active in transferring β-1,4-galactose to terminal GlcNAc in an elegant set of experiments that required first generating a mutant of the Alg1p enzyme that transfers the core or Pauci mannose to the growing N-glycan precursor molecule (Schwientek et al, J. of Biol. Chem. 271: 3398-3405 (1996)). This mutation results in the partial transfer of a GlcNAc₂ truncated N-glycan to proteins, and yields a terminal GlcNAc. Following this, the authors show that human GalTI is capable of transferring galactose in a β-1,4-linkage to this artificial terminal GlcNAc structure. Importantly, it is shown that human GalTI can be expressed in active form in the Golgi of a yeast.

Based upon the above, the present invention was first tested with expression of human GalTI-leader peptide fusion proteins targeted to the Golgi apparatus of the host cell. After subsequent screening of human GalTII, GalTIII, GalTIV, GalTV, Bovine GalTI, and a pair of putative C. elegans GalTs, it was found that human GalTI appeared to be the most active enzyme in transferring galactose to complex biantennary N-glycans in this heterologous system. This may indicate that hGalTI is the most capable enzyme for transferring to this substrate (biantennary complex N-glycan) or it might simply be the most stable and active of the GalT enzymes tested or a combination of both. Interestingly, when GalT was localized to the Golgi apparatus using the same leader peptide used to localize the mannosidase II and GnT II catalytic domain-leader fusion proteins to the Golgi apparatus, a significant percentage of hybrid N-glycan structures (up to 20%) resulted in which a terminal galactose was on the α-1,3 arm. An increase in expression of the mannosidase II or GnT II activity did not significantly reduce this phenomenon. However, screening a library of yeast type II secretory pathway localization leader peptides (peptides that localize to a desired organelle of the secretory pathway such as the ER, Golgi or the trans Golgi network) yielded several active GalT-leader peptide fusion proteins that when transformed into the host cell resulted in significantly reduced levels of hybrid N-glycan structures and an increase in complex N-glycan structures. Previously, it has been reported that bisecting GlcNAc transfer is a stop signal for subsequent sugar transfer in the maturation of N-glycans in the Golgi, including preventing the transfer of fucose (Umana et al., Nature Biotechnol. 17: 176-180 (1999)). The data here suggests that transfer of galactose to the α-1,3 arm results in a similar stop signal preventing maturation of the α-1,6 arm by mannosidase II and GnT II.

The above results suggest that the ratio of galactose-terminated or -containing hybrid N-glycans to galactose-terminated or -containing complex N-glycans produced in a recombinant host cells is a product of where the GalTI is localized in the Golgi apparatus with respect to where the mannosidase II and GnT II are localized and that by manipulating where the three enzymes are localized, the ratio of hybrid N-glycans to complex N-glycans can be manipulated. To increase the yield of galactose-terminated or -containing hybrid N-glycans, all three enzyme activities should be targeted to the same region of the Golgi apparatus, for example, by using the same secretory pathway targeting leader peptide for targeting all three enzyme activities. Alternatively, a library of GalT-leader peptide fusion proteins is screened to identify a fusion protein that places the GalT activity in a position in the Golgi apparatus where it is more likely to act on the N-glycan substrate before the other two enzymes can act. This increases the yield of galactose-terminated or containing hybrid N-glycans compared to galactose-terminated or containing complex N-glycans. Conversely, to reduce the yield of galactose-terminated or -containing hybrid N-glycans, the GalT activity should be localized using a secretory pathway targeting leader peptide that is different from secretory pathway targeting leader peptide that is used with the other two enzyme activities. This can be achieved by screening GalT-leader peptide fusion protein libraries to identify a GalT-leader peptide fusion protein combination that results in a host cell in which the yield of galactose-terminated or containing complex N-glycans is increased compared to the yield of galactose-terminated or containing hybrid N-glycans.

The results further underscore the value of screening libraries of both catalytic domains and secretory pathway localization leader peptides when expressing chimeric glycosylation enzymes in a heterologous host system, a concept previously illustrated by Choi et al., Proc. Natl. Acad. Sci. USA 100: 5022-5027 (2003) and described in U.S. Pat. Nos. 7,029,872 and 7,449,308. With the availability of whole genome sequences, more sophisticated PCR and cloning techniques, and higher throughput analytical techniques like mass spectrometry, screening hundreds even thousands of combinations is possible. Importantly, this type of combinatorial screening has proven to be the difference between detecting enzyme activity and driving stepwise reactions, each dependent on the previous product to completion. Here, while in the case of UDP-galactose 4-epimerase several enzymes screened seemed to have relatively equal abilities to generate a pool of UDP-galactose, only one of the four UDP-galactose transporters tested proved active in this heterologous host including, surprisingly, one inactive transporter from a fellow yeast (S. pombe). Furthermore, from a screen of dozens of secretory pathway localization leader peptides, many of which yielded active enzyme combinations, only three were found that yielded a high degree of uniform complex N-glycans (>85%) with biantennary terminal galactose (G2). Reduction of high mannose and hybrid intermediate structures has important consequences for the use of such a heterologous expression system in the production of therapeutic glycoproteins as high mannose structures have been shown to be potently immunogenic and recent evidence has suggested that liver toxicity can result from an overabundance of hybrid N-glycans.

Thus, U.S. Published application No. 2006/0286637 taught that to achieve galactose transfer in a host cell that does not normally produce glycoproteins that contain galactose, three conditions bad to be overcome: (1) the absence of endogenous galactosyltransferase (GalT) in the Golgi, (2) the absence of endogenous UDP-Gal transport into the Golgi, and (3) a low endogenous cytosolic UDP-Gal pool. In the absence of a UDP-Gal transporter, the transfer of galactose to the terminal GlcNAc residue on the N-glycan was about 55-60%. In the absence of a UDP-glucose-4-epimerase, the transfer of galactose to the terminal GlcNAc residue on the N-glycan was about 10-15%.

All of the above modifications to produce host cells that can make galactosylated N-glycans had been made using a reporter protein (K3 domain of human plasminogen) with exposed N-glycans that are typically sialylated in humans and has been reported in U.S. Published Application No. 20060040353. However, in a manner that is comparable to mammalian cells, these glycoengineered yeast strain yielded only partial galactose transfer onto the Fc glycan of antibodies, resulting in a pool of N-glycans with less than 10% G2 and less than 25% G1 structures in favor of complex N-glycans with terminal GlcNAc. This is similar to mammalian cell lines, where antibody (IgG Fc Asn 297) N-glycans contain reduced amounts of terminal galactose.

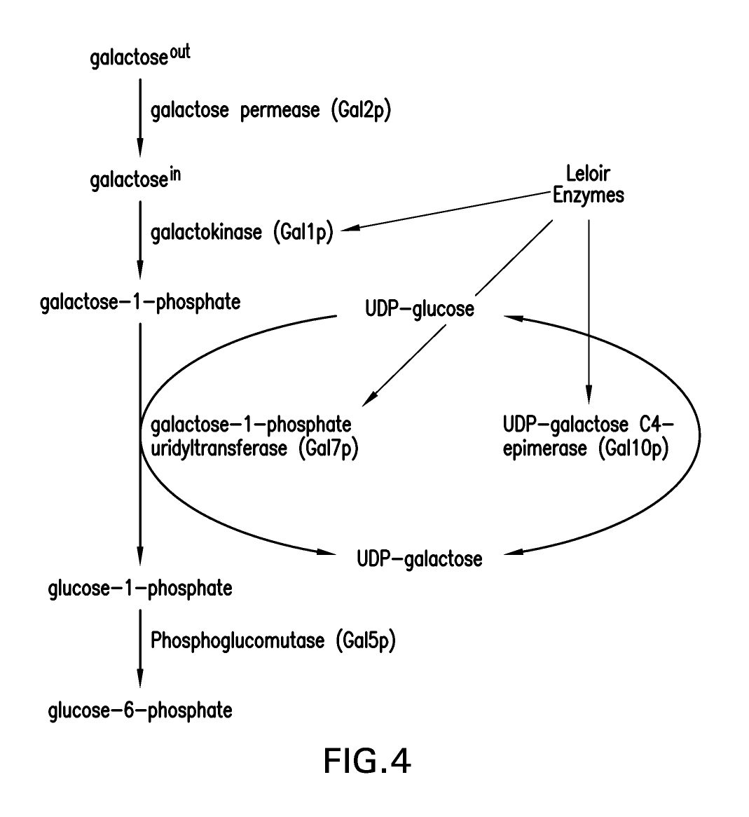

The transfer of galactose residues onto N-glycans requires a pool of activated galactose (UDP-Gal). One way to generate such a pool above endogenous levels in a lower eukaryote is the expression of a UDP-galactose 4 epimerase as stated above and shown in U.S. Published Application No. 2006/0040353. Another way is the present invention, which includes the above cells, wherein the host cells are transformed with nucleic acid molecules encoding at least the following three Leloir pathway enzymes: galactokinase (EC 2.7.1.6), galactose-1-phosphate uridyl transferase EC 2.7.7.12), and UDP-galactose 4 epimerase (EC 5.1.3.2). Galactokinase is an enzyme that catalyzes the first step of galactose metabolism, namely the phosphorylation of galactose to galactose-1-phosphate. Galactose-1-phosphate uridyl transferase catalyzes the second step of galactose metabolism, which is the conversion of UDP-glucose and galactose-1-phosphate to UDP-galactose and glucose-1-phosphate. Optionally, further included can be a nucleic acid molecule encoding a plasma membrane galactose permease. Galactose permease is a plasma membrane hexose transporter, which imports galactose from an exogenous source. The Leloir pathway is shown in FIG. 4.

As shown herein, when the genes encoding a galactokinase activity, a UDP-galactose-4-epimerase activity, a galactose-1-phosphate uridyl transferase activity and optionally a galactose permease activity into the above yeast along with feeding the yeast exogenous galactose are introduced into the cells and antibody expression is induced, the levels of terminal galactose on the Fc N-glycan of the antibody are substantially increased, thus increasing the amount of G2 N-glycans produced: N-glycans could be obtained on a human Fc wherein greater than 50% of the N-glycans were G2. The permease is optional because it was found that endogenous permeases in the cell were capable of importing galactose into the cell. Therefore, when used, the galactose permease can be any plasma membrane hexose transporter capable of transporting galactose across the cell membrane, for example, the GAL2 galactose permease from S. cerevisiae (See GenBank: M81879). The galactokinase can be any enzyme that can catalyze the phosphorylation of galactose to galactose-1-phosphate, for example, the GAL1 gene from S. cerevisiae (See GenBank: X76078). The galactose-1-phosphate uridyl transferase can be any enzyme that catalyzes the conversion of UDP-glucose and galactose-1-phosphate to UDP-galactose and glucose-1-phosphate, for example, the GAL7 of S. cerevisiae (GenBank: See M12348). The UDP-galactose 4 epimerase can be any enzyme that catalyzes the conversion of UDP-glucose to UDP-galactose, for example the GAL10 of S. cerevisiae (See GenBank NC_{—}001134), GALE (See GenBank NC_{—}003423) of S. pombe, and hGALE of human (See GenBank NM_{—}000403). The epimerase can also be provided as a fusion protein in which the catalytic domain of the epimerase is fused to the catalytic domain of a galactosyltransferase (See U.S. Published Application No. US2006/0040353).

Introducing the above Leloir pathway enzymes into a P. pastoris strain produced a recombinant cell that was capable of assimilating environmental galactose. Further, when the above Leloir pathway enzymes are introduced into a cell capable of producing glycoproteins with N-glycans that contain galactose, the resulting recombinant cells were able to produce glycoproteins that had higher levels of β-1,4-galactose on complex N-glycans than glycoproteins produced in cells that had not been so engineered. Thus, the combination of these engineering steps has yielded host cells specifically glycoengineered and metabolically engineered for increased control over glycosylation of the N-glycans of glycoproteins such as antibodies and Fc-fusion proteins. Thus, these glycoengineered yeast cell lines enable efficient production of recombinant antibodies and Fc fusion proteins while allowing control over N-glycan processing.

### Expression Vectors

In general, the galactokinase, UDP-galactose-4-epimerase, and galactoctose-1-phosphate uridyl transferase (and optionally galactose permease) are expressed as components of an expression cassette from an expression vector. In further aspects, further included in the host cell is an expression vector encoding a recombinant protein of interest, which in particular embodiments further includes a sequence that facilitates secretion of the recombinant protein from the host cell. For each Leloir pathway enzyme and recombinant protein of interest, the expression vector encoding it minimally contains a sequence, which affects expression of the nucleic acid sequence encoding the Leloir pathway enzyme or recombinant protein. This sequence is operably linked to a nucleic acid molecule encoding the Leloir pathway enzyme or recombinant protein. Such an expression vector can also contain additional elements like origins of replication, selectable markers, transcription or termination signals, centromeres, autonomous replication sequences, and the like.

According to the present invention, nucleic acid molecules encoding a recombinant protein of interest and the above Leloir pathway enzymes, respectively, can be placed within expression vectors to permit regulated expression of the overexpressed recombinant protein of interest and the above Leloir pathway enzymes. While the recombinant protein and the above Leloir pathway enzymes can be encoded in the same expression vector, the above Leloir pathway enzymes are preferably encoded in an expression vector which is separate from the vector encoding the recombinant protein. Placement of nucleic acid molecules encoding the above Leloir pathway enzymes and the recombinant protein in separate expression vectors can increase the amount of recombinant protein produced.

As used herein, an expression vector can be a replicable or a non-replicable expression vector. A replicable expression vector can replicate either independently of host cell chromosomal DNA or because such a vector has integrated into host cell chromosomal DNA. Upon integration into host cell chromosomal DNA such an expression vector can lose some structural elements but retains the nucleic acid molecule encoding the recombinant protein or the above Leloir pathway enzymes and a segment which can effect expression of the recombinant protein or the above Leloir pathway enzymes. Therefore, the expression vectors of the present invention can be chromosomally integrating or chromosomally nonintegrating expression vectors.

Following introduction of nucleic acid molecules encoding the above Leloir pathway enzymes and the recombinant protein, the recombinant protein is then overexpressed by inducing expression of the nucleic acid encoding the recombinant protein. In another embodiment, cell lines are established which constitutively or inducibly express the above Leloir pathway enzymes. An expression vector encoding the recombinant protein to be overexpressed is introduced into such cell lines to achieve increased production of the recombinant protein. In particular embodiments, the nucleic acid molecules encoding the Leloir pathway enzymes are operably linked to constitutive promoters.

The present expression vectors can be replicable in one host cell type, e.g., Escherichia coli, and undergo little or no replication in another host cell type, e.g., a eukaryotic host cell, so long as an expression vector permits expression of the above Leloir pathway enzymes or overexpressed recombinant protein and thereby facilitates secretion of such recombinant proteins in a selected host cell type.

Expression vectors as described herein include DNA or RNA molecules engineered for controlled expression of a desired gene, that is, genes encoding the above Leloir pathway enzymes or recombinant protein. Such vectors also encode nucleic acid molecule segments which are operably linked to nucleic acid molecules encoding the present above Leloir pathway enzymes or recombinant protein. Operably linked in this context means that such segments can effect expression of nucleic acid molecules encoding above Leloir pathway enzymes or recombinant protein. These nucleic acid sequences include promoters, enhancers, upstream control elements, transcription factors or repressor binding sites, termination signals and other elements which can control gene expression in the contemplated host cell. Preferably the vectors are vectors, bacteriophages, cosmids, or viruses.

Expression vectors of the present invention function in yeast or mammalian cells. Yeast vectors can include the yeast 2μ circle and derivatives thereof, yeast vectors encoding yeast autonomous replication sequences, yeast minichromosomes, any yeast integrating vector and the like. A comprehensive listing of many types of yeast vectors is provided in Parent et al. (Yeast 1:83-138 (1985)).

Elements or nucleic acid sequences capable of effecting expression of a gene product include promoters, enhancer elements, upstream activating sequences, transcription termination signals and polyadenylation sites. All such promoter and transcriptional regulatory elements, singly or in combination, are contemplated for use in the present expression vectors. Moreover, genetically-engineered and mutated regulatory sequences are also contemplated herein.

Promoters are DNA sequence elements for controlling gene expression. In particular, promoters specify transcription initiation sites and can include a TATA box and upstream promoter elements. The promoters selected are those which would be expected to be operable in the particular host system selected. For example, yeast promoters are used in the present expression vectors when a yeast host cell such as Saccharomyces cerevisiae, Kluyveromyces lactis, or Pichia pastoris is used whereas fungal promoters would be used in host cells such as Aspergillus niger, Neurospora crassa, or Tricoderma reesei. Examples of yeast promoters include but are not limited to the GAPDH, AOX1, SEC4, HH1, PMA1, OCH1, GAL1, PGK, GAP, TP1, CYC1, ADH2, PH05, CUP1, MFα1, FLD1, PMA1, PDI, TEF, and GUT1 promoters. Romanos et al. (Yeast 8: 423-488 (1992)) provide a review of yeast promoters and expression vectors. Hartner et al., Nucl. Acid Res. 36: e76 (pub on-line 6 Jun. 2008) describes a library of promoters for fine-tuned expression of heterologous proteins in Pichia pastoris.

The promoters that are operably linked to the nucleic acid molecules disclosed herein can be constitutive promoters or inducible promoters. Inducible promoters, that is promoters which direct transcription at an increased or decreased rate upon binding of a transcription factor. Transcription factors as used herein include any factor that can bind to a regulatory or control region of a promoter and thereby affect transcription. The synthesis or the promoter binding ability of a transcription factor within the host cell can be controlled by exposing the host to an inducer or removing an inducer from the host cell medium. Accordingly, to regulate expression of an inducible promoter, an inducer is added or removed from the growth medium of the host cell. Such inducers can include sugars, phosphate, alcohol, metal ions, hormones, heat, cold and the like. For example, commonly used inducers in yeast are glucose, galactose, and the like.

Transcription termination sequences that are selected are those that are operable in the particular host cell selected. For example, yeast transcription termination sequences are used in the present expression vectors when a yeast host cell such as Saccharomyces cerevisiae, Kluyveromyces lactis, or Pichia pastoris is used whereas fungal transcription termination sequences would be used in host cells such as Aspergillus niger, Neurospora crassa, or Tricoderma reesei. Transcription termination sequences include but are not limited to the Saccharomyces cerevisiae CYC transcription termination sequence (ScCYC TT), the Pichia pastoris ALG3 transcription termination sequence (ALG3 TT), the Pichia pastoris ALG6 transcription termination sequence (ALG6 TT), the Pichia pastoris ALG12 transcription termination sequence (ALG12 TT), the Pichia pastoris AOX1 transcription termination sequence (AOX1 TT), the Pichia pastoris OCH1 transcription termination sequence (OCH1 TT) and Pichia pastoris PMA1 transcription termination sequence (PMA1 TT).

The expression vectors of the present invention can also encode selectable markers. Selectable markers are genetic functions that confer an identifiable trait upon a host cell so that cells transformed with a vector carrying the selectable marker can be distinguished from non-transformed cells. Inclusion of a selectable marker into a vector can also be used to ensure that genetic functions linked to the marker are retained in the host cell population. Such selectable markers can confer any easily identified dominant trait, e.g. drug resistance, the ability to synthesize or metabolize cellular nutrients and the like.

Yeast selectable markers include drug resistance markers and genetic functions which allow the yeast host cell to synthesize essential cellular nutrients, e.g. amino acids. Drug resistance markers which are commonly used in yeast include chloramphenicol, kanamycin, methotrexate, G418 (geneticin), Zeocin, and the like. Genetic functions which allow the yeast host cell to synthesize essential cellular nutrients are used with available yeast strains having auxotrophic mutations in the corresponding genomic function. Common yeast selectable markers provide genetic functions for synthesizing leucine (LEU2), tryptophan (TRP1 and TRP2), uracil (URA3, URA5, URA6), histidine (HIS3), lysine (LYS2), adenine (ADE1 or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al., J. Biol. Chem. 272:30061-30066 (1997)). A number of suitable integration sites include those enumerated in U.S. Published application No. 2007/0072262 and include homologs to loci known for Saccharomyces cerevisiae and other yeast or fungi. Methods for integrating vectors into yeast are well known, for example, see WO2007136865.

Therefore the present expression vectors can encode selectable markers which are useful for identifying and maintaining vector-containing host cells within a cell population present in culture. In some circumstances selectable markers can also be used to amplify the copy number of the expression vector. After inducing transcription from the present expression vectors to produce an RNA encoding an overexpressed recombinant protein or Leloir pathway enzymes, the RNA is translated by cellular factors to produce the recombinant protein or Leloir pathway enzymes.

In yeast and other eukaryotes, translation of a messenger RNA (mRNA) is initiated by ribosomal binding to the 5′ cap of the mRNA and migration of the ribosome along the mRNA to the first AUG start codon where polypeptide synthesis can begin. Expression in yeast and mammalian cells generally does not require specific number of nucleotides between a ribosomal-binding site and an initiation codon, as is sometimes required in prokaryotic expression systems. However, for expression in a yeast or a mammalian host cell, the first AUG codon in an mRNA is preferably the desired translational start codon.

Moreover, when expression is performed in a yeast host cell the presence of long untranslated leader sequences, e.g. longer than 50-100 nucleotides, can diminish translation of an mRNA. Yeast mRNA leader sequences have an average length of about 50 nucleotides, are rich in adenine, have little secondary structure and almost always use the first AUG for initiation. Since leader sequences which do not have these characteristics can decrease the efficiency of protein translation, yeast leader sequences are preferably used for expression of an overexpressed gene product or a chaperone protein in a yeast host cell. The sequences of many yeast leader sequences are known and are available to the skilled artisan, for example, by reference to Cigan et al. (Gene 59: 1-18 (1987)).

In addition to the promoter, the ribosomal-binding site and the position of the start codon, factors which can affect the level of expression obtained include the copy number of a replicable expression vector. The copy number of a vector is generally determined by the vector's origin of replication and any cis-acting control elements associated therewith. For example, an increase in copy number of a yeast episomal vector encoding a regulated centromere can be achieved by inducing transcription from a promoter which is closely juxtaposed to the centromere. Moreover, encoding the yeast FLP function in a yeast vector can also increase the copy number of the vector.

One skilled in the art can also readily design and make expression vectors which include the above-described sequences by combining DNA fragments from available vectors, by synthesizing nucleic acid molecules encoding such regulatory elements or by cloning and placing new regulatory elements into the present vectors. Methods for making expression vectors are well-known. Overexpressed DNA methods are found in any of the myriad of standard laboratory manuals on genetic engineering.

The expression vectors of the present invention can be made by ligating the coding regions for the above Leloir pathway enzymes and recombinant protein in the proper orientation to the promoter and other sequence elements being used to control gene expression. After construction of the present expression vectors, such vectors are transformed into host cells where the overexpressed recombinant protein and the Leloir pathway enzymes can be expressed. Methods for transforming yeast and other lower eukaryotic cells with expression vectors are well known and readily available to the skilled artisan. For example, expression vectors can be transformed into yeast cells by any of several procedures including lithium acetate, spheroplast, electroporation, and similar procedures.

### Host Cells

Yeast such as Pichia pastoris, Pichia methanolica, and Hansenula polymorpha are useful for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale. In general, lower eukaryotes useful for practicing the methods herein include yeast and fungi that cannot normally use galactose as a carbon source. Examples of yeast that cannot use galactose as a carbon source include but are not limited to methylotrophic yeast of the Pichia genus, e.g., Pichia pastoris, and yeast such as S. kudriavzevii, C. glabrata, K waltii, and E. gossypii. Yeast are useful for expression of glycoproteins because they can be economically cultured, give high yields, and when appropriately modified are capable of suitable glycosylation. Yeast particularly offers established genetics allowing for rapid transformations, tested protein localization strategies and facile gene knock-out techniques. Suitable vectors have expression control sequences, such as promoters, including 3-phosphoglycerate kinase or other glycolytic enzymes, and an origin of replication, termination sequences and the like as desired. Thus, the above host cells, which cannot normally use galactose as a carbon source, are genetically engineered to express a galactokinase activity, a UDP-galactose-4-epimerase activity, a galactoctose-1-phosphate uridyl transferase activity and optionally a galactose permease activity, which renders the host cells capable of using galactose as a carbon source.

Lower eukaryotes, particularly yeast, can also be genetically modified so that they express glycoproteins in which the glycosylation pattern is human-like or humanized. Such can be achieved by eliminating selected endogenous glycosylation enzymes and/or supplying exogenous enzymes as described by Gerngross et al., U.S. Published Application No. 2004/0018590. For example, a host cell can be selected or engineered to be depleted in 1,6-mannosyl transferase activities, which would otherwise add mannose residues onto the N-glycan on a glycoprotein.

In one embodiment, the host cells genetically engineered to assimilate environmental galactose as a carbon source as described herein is also genetically engineered to make complex N-glycans as described below. Such host cells further includes an α-1,2-mannosidase catalytic domain fused to a cellular targeting signal peptide not normally associated with the catalytic domain and selected to target the α-1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform. For example, U.S. Pat. No. 7,029,872 and U.S. Published Patent Application Nos. 2004/0018590 and 2005/0170452 disclose lower eukaryote host cells capable of producing a glycoprotein comprising a Man₅GlcNAc₂ glycoform. These host cells when further genetically engineered to express a galactokinase activity, a UDP-galactose-4-epimerase activity, a galactoctose-1-phosphate uridyl transferase activity and optionally a galactose permease activity as taught herein are capable of using galactose as a carbon source.

The immediately preceding host cell further includes a GlcNAc transferase I (GnT I) catalytic domain fused to a cellular targeting signal peptide not normally associated with the catalytic domain and selected to target the GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example a recombinant glycoprotein composition comprising predominantly a GlcNAcMan₅GlcNAc₂ glycoform. U.S. Pat. No. 7,029,872 and U.S. Published Patent Application Nos. 2004/0018590 and 2005/0170452 disclose lower eukaryote host cells capable of producing a glycoprotein comprising a GlcNAcMan₅GlcNAc₂ glycoform. The glycoprotein produced in the above cells can be treated in vitro with a hexaminidase to produce a recombinant glycoprotein comprising a Man₅GlcNAc₂ glycoform. These host cells when further genetically engineered to express a galactokinase activity, a UDP-galactose-4-epimerase activity, a galactoctose-1-phosphate uridyl transferase activity and optionally a galactose permease activity as taught herein are capable of using galactose as a carbon source.

Then, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Published Patent Application No. 2004/0230042 discloses lower eukaryote host cells that express mannosidase II enzymes and are capable of producing glycoproteins having predominantly a GlcNAc₂Man₃GlcNAc₂ glycoform. The glycoprotein produced in the above cells can be treated in vitro with a hexaminidase to produce a recombinant glycoprotein comprising a Man₃GlcNAc₂ glycoform. These host cells when further genetically engineered to express a galactokinase activity, a UDP-galactose-4-epimerase activity, a galactoctose-1-phosphate uridyl transferase activity and optionally a galactose permease activity as taught herein are capable of using galactose as a carbon source.

Then, the immediately preceding host cell further includes GlcNAc transferase H (GnT II) catalytic domain fused to a cellular targeting signal peptide not normally associated with the catalytic domain and selected to target the GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ (G0) glycoform, for example a recombinant glycoprotein composition comprising predominantly a GlcNAc₂Man₃GlcNAc₂ glycoform. U.S. Pat. No. 7,029,872 and U.S. Published Patent Application Nos. 2004/0018590 and 2005/0170452 disclose lower eukaryote host cells capable of producing a glycoprotein comprising a GlcNAc₂Man₃GlcNAc₂ glycoform. The glycoprotein produced in the above cells can be treated in vitro with a hexaminidase to produce a recombinant glycoprotein comprising a Man₃GlcNAc₂ glycoform. These host cells when further genetically engineered to express a galactokinase activity, a UDP-galactose-4-epimerase activity, a galactoctose-1-phosphate uridyl transferase activity and optionally a galactose permease activity as taught herein are capable of using galactose as a carbon source.

Finally, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂Man₃GlcNAc₂ (G1) or Gal₂GlcNAc₂Man₃GlcNAc₂ (G2) glycoform, or mixture thereof for example a recombinant glycoprotein composition comprising predominantly a GalGlcNAc₂Man₃GlcNAc₂ (G1) glycoform or Gal₂GlcNAc₂Man₃GlcNAc₂ (G2) glycoform or mixture thereof. U.S. Pat. No. 7,029,872 and U.S. Published Patent Application No. 2006/0040353 discloses lower eukaryote host cells capable of producing a glycoprotein comprising a Gal₂GlcNAc₂Man₃GlcNAc₂ glycoform. The glycoprotein produced in the above cells can be treated in vitro with a galactosidase to produce a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. These host cells when further genetically engineered to express a galactokinase activity, a UDP-galactose-4-epimerase activity, a galactoctose-1-phosphate uridyl transferase activity and optionally a galactose permease activity as taught herein are capable of using galactose as a carbon source and are capable of producing glycoproteins wherein the proportion of N-glycans containing galactose is greater than in host cells that have not been genetically engineered to include the above-mention Leloir pathway enzymes.

In a further embodiment, the immediately preceding host cell, which is capable of making complex N-glycans terminated with galactose and which is capable of assimilating galactose as a carbon source as disclosed herein, can further include a sialyltransferase catalytic domain fused to a cellular targeting signal peptide not normally associated with the catalytic domain and selected to target sialytransferase activity to the ER or Golgi apparatus of the host cell. Passage of the recombinant glycoprotein through the ER or Golgi apparatus of the host cell produces a recombinant glycoprotein comprising predominantly a NANA₂Gal₂GlcNAc₂Man₃GlcNAc₂ glycoform or NANAGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Published Patent Application No. 2005/0260729 discloses a method for genetically engineering lower eukaryotes to have a CMP-sialic acid synthesis pathway and U.S. Published Patent Application No. 2006/0286637 discloses a method for genetically engineering lower eukaryotes to produce sialylated glycoproteins. These host cells when further genetically engineered to express a galactokinase activity, a UDP-galactose-4-epimerase activity, a galactoctose-1-phosphate uridyl transferase activity and optionally a galactose permease activity as taught herein are capable of using galactose as a carbon source and are capable of producing glycoproteins wherein the proportion of N-glycans containing galactose is greater than in host cells that have not been genetically engineered to include the above-mention Leloir pathway enzymes.

In another embodiment, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target Galactosyltransferase activity to the ER or Golgi apparatus of the host cell. Passage of the recombinant glycoprotein through the ER or Golgi apparatus of the host cell produces a recombinant glycoprotein comprising predominantly the GalGlcNAcMan₅GlcNAc₂ glycoform. These host cells when further genetically engineered to express a galactokinase activity, a UDP-galactose-4-epimerase activity, a galactoctose-1-phosphate uridyl transferase activity and optionally a galactose permease activity as taught herein are capable of using galactose as a carbon source.

In a further embodiment, the immediately preceding host cell, which is capable of making hybrid N-glycans terminated with galactose and which is capable of assimilating galactose as a carbon source as disclosed herein, can further include a sialyltransferase catalytic domain fused to a cellular targeting signal peptide not normally associated with the catalytic domain and selected to target sialytransferase activity to the ER or Golgi apparatus of the host cell. Passage of the recombinant glycoprotein through the ER or Golgi apparatus of the host cell produces a recombinant glycoprotein comprising a NANAGalGlcNAcMan₅GlcNAc₂ glycoform. These host cells when further genetically engineered to express a galactokinase activity, a UDP-galactose-4-epimerase activity, a galactoctose-1-phosphate uridyl transferase activity and optionally a galactose permease activity as taught herein are capable of using galactose as a carbon source.

Various of the preceding host cells further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

Host cells further include the cells that are genetically engineered to eliminate glycoproteins having α-mannosidase-resistant N-glycans by deleting or disrupting one or more of the β-mannosyltransferase genes (e.g., BMT1, BMT2, BMT3, and BMT4) (See, U.S. Published Patent Application No. 2006/0211085) and glycoproteins having phosphomannose residues by deleting or disrupting one or both of the phosphomannosyl transferase genes PNO1 and MNN4B

(See for example, U.S. Pat. Nos. 7,198,921 and 7,259,007), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. The host cells can further include any one of the aforementioned host cells modified to produce particular N-glycan structures.

Host cells further include lower eukaryote cells (e.g., yeast such as Pichia pastoris) that are genetically modified to control β-glycosylation of the glycoprotein by deleting or disrupting one or more of the protein O-mannosyltransferase (Dol-P-Man:Protein (Ser/Thr)

Mannosyl Transferase genes) (PMTS) (See U.S. Pat. No. 5,714,377) or grown in the presence of Pmtp inhibitors and/or an alpha-mannosidase as disclosed in Published International Application No. WO 2007061631, or both. Disruption includes disrupting the open reading frame encoding the Pmtp or disrupting expression of the open reading frame or abrogating translation of RNAs encoding one or more of the Pmtps using interfering RNA, antisense RNA, or the like. The host cells can further include any one of the aforementioned host cells modified to produce particular N-glycan structures.

Pmtp inhibitors include but are not limited to a benzylidene thiazolidinediones. Examples of benzylidene thiazolidinediones that can be used are 5-[[3,4-bis(phenylmethoxy)phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic Acid.

In particular embodiments, the function or expression of at least one endogenous PMT gene is reduced, disrupted, or deleted. For example, in particular embodiments the function or expression of at least one endogenous PMT gene selected from the group consisting of the PMT1, PMT2, PMT3, and PMT4 genes is reduced, disrupted, or deleted; or the host cells are cultivated in the presence of one or more PMT inhibitors. In further embodiments, the host cells include one or more PMT gene deletions or disruptions and the host cells are cultivated in the presence of one or more Pmtp inhibitors. In particular aspects of these embodiments, the host cells also express a secreted alpha-1,2-mannosidase.

PMT deletions or disruptions and/or Pmtp inhibitors control O-glycosylation by reducing O-glycosylation occupancy, that is by reducing the total number of O-glycosylation sites on the glycoprotein that are glycosylated. The further addition of an alpha-1,2-mannosidase that is secreted by the cell controls O-glycosylation by reducing the mannose chain length of the O-glycans that are on the glycoprotein. Thus, combining PMT deletions or disruptions and/or Pmtp inhibitors with expression of a secreted alpha-1,2-mannosidase controls O-glycosylation by reducing occupancy and chain length. In particular circumstances, the particular combination of PMT deletions or disruptions, Pmtp inhibitors, and alpha-1,2-mannosidase is determined empirically as particular heterologous glycoproteins (Fabs and antibodies, for example) may be expressed and transported through the Golgi apparatus with different degrees of efficiency and thus may require a particular combination of PMT deletions or disruptions, Pmtp inhibitors, and alpha-1,2-mannosidase. In another aspect, genes encoding one or more endogenous mannosyltransferase enzymes are deleted. This deletion(s) can be in combination with providing the secreted alpha-1,2-mannosidase and/or PMT inhibitors or can be in lieu of providing the secreted alpha-1,2-mannosidase and/or PMT inhibitors.

Thus, the control of O-glycosylation can be useful for producing particular glycoproteins in the host cells disclosed herein in better total yield or in yield of properly assembled glycoprotein. The reduction or elimination of O-glycosylation appears to have a beneficial effect on the assembly and transport of whole antibodies and Fab fragments as they traverse the secretory pathway and are transported to the cell surface. Thus, in cells in which O-glycosylation is controlled, the yield of properly assembled antibodies or Fab fragments is increased over the yield obtained in host cells in which O-glycosylation is not controlled.

Thus, contemplated are host cells that have been genetically modified to produce glycoproteins wherein the predominant N-glycans thereon include but are not limited to Man₈GlcNAc₂, Man₇GlcNAc₂, Man₆GlcNAc₂, Man₅GlcNAc₂, GlcNAcMan₅GlcNAc₂, GalGlcNAcMan₅GlcNAc₂, NANAGalGlcNAcMan₅GlcNAc₂, Man₃GlcNAc₂, GlcNAc₍₁₋₄₎Man₃GlcNAc₂, Gal₍₁₋₄₎GlcNAc₍₁₋₄₎Man₃GlcNAc₂, NANA₍₁₋₄₎Gal₍₁₋₄₎GlcNAc₍₁₋₄₎Man₃GlcNAc₂. Further included are host cells that produce glycoproteins that have particular mixtures of the aforementioned N-glycans thereon.

The host cells and methods herein are useful for producing a wide range of recombinant protein and glycoproteins. Examples of recombinant proteins and glycoproteins that can be produced in the host cells disclosed herein include but are not limited to erythropoietin (EPO); cytokines such as interferon α, interferon β, interferon γ, and interferon ω; and granulocyte-colony stimulating factor (GCSF); GM-CSF; coagulation factors such as factor VIII, factor IX, and human protein C; antithrombin III; thrombin,; soluble IgE receptor α-chain; immunoglobulins such as IgG, IgG fragments, IgG fusions, and IgM; immunoadhesions and other Fc fusion proteins such as soluble TNF receptor-Fc fusion proteins; RAGE-Fc fusion proteins; interleukins; urokinase; chymase; and urea trypsin inhibitor; IGF-binding protein; epidermal growth factor; growth hormone-releasing factor; annexin V fusion protein; angiostatin; vascular endothelial growth factor-2; myeloid progenitor inhibitory factor-1; osteoprotegerin; α-1-antitrypsin; α-feto proteins; DNase II; kringle 3 of human plasminogen; glucocerebrosidase; TNF binding protein 1; follicle stimulating hormone; cytotoxic T lymphocyte associated antigen 4-Ig; transmembrane activator and calcium modulator and cyclophilin ligand; glucagon like protein 1; and IL-2 receptor agonist.

The recombinant host cells of the present invention disclosed herein are particularly useful for producing antibodies, Fc fusion proteins, and the like where it is desirable to provide antibody compositions wherein the percent galactose-containing N-glycans is increased compared to the percent galactose obtainable in the host cells prior to modification as taught herein. In general, the host cells enable antibody compositions to be produced wherein the ratio of G0:G1/G2 glycoforms is less than 2:1. Examples of antibodies that can be made in the host cells herein and have a ratio of G0:G1/G2 of less than 2:1 include but are not limited to human antibodies, humanized antibodies, chimeric antibodies, heavy chain antibodies (e.g., camel or llama). Specific antibodies include but are not limited to the following antibodies recited under their generic name (target): Muromonab-CD3 (anti-CD3 receptor antibody), Abciximab (anti-CD41 7E3 antibody), Rituximab (anti-CD20 antibody), Daelizumab (anti-CD25 antibody), Basiliximab (anti-CD25 antibody), Palivizumab (anti-RSV (respiratory syncytial virus) antibody), Infliximab (anti-TNFα antibody), Trastuzumab (anti-Her2 antibody), Gemtuzumab ozogamicin (anti-CD33 antibody), Alemtuzumab (anti-CD52 antibody), Ibritumomab tiuxeten (anti-CD20 antibody), Adalimumab (anti-TNFα antibody), Omalizumab (anti-IgE antibody), Tositumomab-¹³¹I (iodinated derivative of an anti-CD20 antibody), Efalizumab (anti-CD11a antibody), Cetuximab (anti-EGF receptor antibody), Golimumab (anti-TNFα antibody), Bevacizumab (anti VEGF-A antibody), and variants thereof. Examples of Fc-fusion proteins that can be made in the host cells disclosed herein include but are not limited to etancercept (TNFR-Fc fusion protein), FGF-21-Fc fusion proteins, GLP-1-Fc fusion proteins, RAGE-Fc fusion proteins, EPO-Fc fusion proteins, ActRIIA-Fc fusion proteins, ActRIIB-Fc fusion proteins, glucagon-Fc fusions, oxyntomodulin-Fc-fusions, and analogs and variants thereof.

The recombinant cells disclosed herein can be used to produce antibodies and Fc fragments suitable for chemically conjugating to a heterologous peptide or drug molecule. For example, WO2005047334, WO2005047336, WO2005047337, and WO2006107124 discloses chemically conjugating peptides or drug molecules to Fc fragments. EP1180121, EP1105409, and U.S. Pat. No. 6,593,295 disclose chemically conjugating peptides and the like to blood components, which includes whole antibodies.

The host cells and/or plasmid vectors encoding various combinations of the Leloir pathway enzymes as taught herein can be provided as kits that provide a selection system for making recombinant Pichia pastoris that express heterologous proteins. The Pichia pastoris host cell is genetically engineered to express one or two of the Leloir pathway enzymes selected from the group consisting of galactokinase, UDP-galactose-4-epimerase, and galactose-1-phosphate uridyl transferase. Optionally, the host cell can express a galactose permease as well. The cloning vector comprises a multiple cloning site and an expression cassette encoding the Leloir pathway enzyme or enzymes not in the provided host cell. The vector can further comprise a Pichia pastoris operable promoter and transcription termination sequence flanking the multiple cloning site and can further comprise a targeting sequence for targeting the vector to a particular location in the host cell genome. In some embodiments, the kit provides a vector that encodes all three Leloir pathway enzymes (galactokinase, UDP-galactose-4-epimerase, and galactose-1-phosphate uridyl transferase) and includes a multiple cloning site and a host cell that lacks the three Leloir pathway enzymes. The kit will further include instructions, vector maps, and the like.

The following examples are intended to promote a further understanding of the present invention.

### Example 1

In this example, a Pichia pastoris host cell capable of producing galactose-containing N-glycans was constructed in general following the methods disclosed in Davidson et al. in U.S. Published Application No. 2006/0040353. The methods herein can be used to make recombinant host cells of other species that are normally incapable of using galactose as a carbon source into a recombinant host cell that is capable of using galactose as a sole carbon source.

The Galactosyltransferase I chimeric enzyme. The Homo sapiens β-1,4-galactosyltransferase I gene (hGalTI, Genbank AH003575) was PCR amplified from human kidney cDNA (Clontech) using PCR primers RCD192 (5′-GCCGCGACCTGAGCC GCCTGCCCCAAC-3′ (SEQ ID NO:1)) and RCD186 (5′-CTAGCTCGGTGTCCCGATGTCCACTGT-3′ (SEQ ID NO:2)). This PCR product was cloned into the pCR2.1 vector (Invitrogen) and sequenced. From this clone, a PCR overlap mutagenesis was performed for three purposes: 1) to remove a NotI site within the open reading frame while maintaining the wild-type protein sequence, 2) to truncate the protein immediately downstream of the endogenous transmembrane domain to provide only the catalytic domain, and 3) to introduce AscI and PacI sites at the 5′ and 3′ ends, respectively, for modular cloning. To do this, the 5′ end of the gene up to the NotI site was PCR amplified using PCR primers RCD198 (5′-CTTAGGCGCGCCGGCCGCGACCTGAGCCGCCTGCCC-3′ (SEQ ID NO:3)) and RCD201 (5′-GGGGCATATCTGCCGCCCATC-3′ (SEQ ID NO:4)) and the 3′ end was PCR amplified with PCR primers RCD200 (5′-GATGGGCGGCAGATATGCCCC-3′ (SEQ ID NO:5)) and RCD199 (5′-CTTCTTAATTAACTAGCTCGGTGTCCCGATGTCCAC-3′ (SEQ ID NO:6)). The products were overlapped together with primers RCD198 and RCD199 to re-synthesize the truncated open reading frame (ORE) encoding the galactosyltransferase with the wild-type amino acid sequence while eliminating the NotI site. The new hGalTIβ PCR catalytic domain product was cloned into the pCR2.1 vector (Invitrogen, Carlsbad, Calif.) and sequenced. The introduced AscI and PacI sites were cleaved with their cognizant restriction enzyme and the DNA fragment subcloned into plasmid pRCD259 downstream of the PpGAPDH promoter to create plasmid pRCD260. The nucleotide sequence encoding the hGalTIβ43 catalytic domain (lacking the first 43 amino acids; SEQ ID NO:50) is shown in SEQ ID NO:49.

A library of yeast leader sequences from S. cerevisiae, P. pastoris, and K. lactis that target proteins to various location in the Golgi was then ligated into this vector between the NotI and AscI sites, thus fusing these leader encoding sequences in-frame with the open reading frame encoding the hGalTIβ43 catalytic domain. The above described combinatorial library of GalT fusion proteins was expressed in YSH44 and the resulting transformants were analyzed by releasing the N-glycans from purified K3 from each transformant and determining their respective molecular mass by MALDI-TOF MS. The P. pastoris strain YSH44 expresses the kringle 3 domain of human plasminogen (K3) as a virtually uniform complex glycoform with bi-antennary terminal GlcNAc residues (GlcNAc₂Man₃GlcNAc₂, See FIG. 1). One of the active constructs was Mnn2-hGalTIβ43, which encoded a fusion protein comprising the N-terminus of S. cerevisiae Mnn2 targeting peptide (amino acids 1-36 (53) SEQ ID NO:20) fused to the N-terminus of the hGalTIβ43 catalytic domain (amino acids 44-398; SEQ ID NO:50). The leader sequence contained the first 108 by of the S. cerevisiae MNN2 gene and it was this sequence that had been inserted between the NotI and AscI sites of pRCD260 to create plasmid pXB53. Plasmid pXB53 was linearized with XbaI. and transformed into yeast strain YSH44 to generate strain YSH71. Strain YSH44 has been described in U.S. Published Application Nos. 20070037248, 20060040353, 20050208617, and 20040230042 and Strain YSH71 has been described in U.S. Published Application No. 20060040353.

As shown in FIG. 3C, a minor portion (about 10%) of the N-glycans produced by strain YSH71 was of a mass consistent with the addition of a single galactose sugar to the GlcNAc₂Man₃GlcNAc₂ (G0) N-glycan substrate on the K3 to make a GalGlcNAc₂Man₃GlcNAc₂ (G1) N-glycan, while the remainder of the N-glycans are identical to the N-glycans produced in the parent strain YSH44 (FIG. 3B). FIG. 3A shows the N-glycans produced in wild-type yeast.

We considered several explanations for the incomplete galactose transfer. These explanations included poor UDP-galactose transport, low endogenous levels of UDP-galactose, or suboptimal GalT activity. It appears that the transfer galactose onto N-glycans might be low because the strains might require a transporter to translocate UDP-galactose from the cytosol to the Golgi apparatus (Ishida et al., J. Biochem. 120: 1074-1078 (1996); Miura et al., J. Biochem (Tokyo) 120: 236-241 (1996); Tabuchi et al., Biochem. Biophys. Res. Corn. 232: 121-125 (1997); Segawa et al., FEBS Letts. 451: 295-298 (1999)). Transporters are complex proteins with multiple transmembrane domains that may not localize properly in a heterologous host. However, several sugar nucleotide transporters, including UDP-galactose transporters have been actively expressed in heterologous systems (Sun-Wada et al., J. Biochem. (Tokyo) 123: 912-917 (1998); Segawa et al. Eur. J. Biochem. 269: 128-138 (2002); Kainuma et al., Glycobiol. 9: 133-141 (1999); Choi et al., Proc Natl Acad Sci USA 100(9): 5022-5027 (2003)). To ensure efficient transport of UDP-galactose into the Golgi, the Drosophila melanogaster gene encoding a UDP-galactose transporter, DmUGT (GenBank accession no. AB055493), was cloned and the clone transformed into strain YSH71 expressing the MNN2-hGalTIβ43 construct as follows.

Cloning of UDP-galactose Transporter. The D. melanogaster gene encoding the UDP Galactose Transporter (GenBank AB055493) referred to as DmUGT was PCR amplified from a D. melanogaster cDNA library (UC Berkeley Drosophila Genome Project, ovary 2,-ZAP library GM) using PCR primers DmUGT-5′ (5′-GGCTCGAGCGGC CGCCACCATGAATAGCATACACATGAACGCCAATACG-3′ (SEQ ID NO:7)) and DmUGT-3′ (5′-CCCTCGAGTTAATTAACTAGACGCGCGGCAGCAGCTTCTCCTCATCG-3′ (SEQ ID NO:8)) and the PCR amplified DNA fragment was cloned into pCR2.1 (Invitrogen, Carlsbad, Calif.) and sequenced. The NotI and PacI sites were then used to subclone this open reading frame into plasmid pRCD393 downstream of the PpOCH1 promoter between the NotI and PacI sites to create plasmid pSH263. The nucleotide sequence encoding the DmUGT is shown in SEQ ID NO:37 and the amino acid sequence of the DmUGT is shown in SEQ ID NO:38. This plasmid was linearized with AgeI and transformed into strain YSH71 to generate strain YSH80. However, no significant change in the N-glycan profile of K3 was found when the plasmid encoding the DmUGT was transformed into YSH71. Therefore, we decided to focus our efforts on enhancing the intracellular pool of UDP-galactose.

Because P. pastoris cannot assimilate galactose as a carbon source (Kurtzman Pichia. The Yeasts: A Taxonomic Study. C. P. a. F. Kurtzman, J. W. Amsterdam, Elsevier Science Publ.: 273-352 (1998)), we speculated that the pool of UDP-galactose in the strain might not be sufficient. The enzyme UDP-galactose 4-epimerase, which is conserved among galactose assimilating organisms, including bacteria and mammals, catalyzes the 3″ step of the Leloir pathway. This enzyme is typically localized in the cytosol of eukaryotes and is responsible for the reversible conversion of UDP-glucose and UDP-galactose (Allard et al., Cell. Mal. Life Sci. 58: 1650-1665 (2001)). We reasoned that expression of a heterologous UDP-galactose 4-epimerase would generate a cytosolic UDP-galactose pool that upon transport into the Golgi would allow the galactose transferase to transfer galactose onto N-glycans.

Cloning of UDP-galactose 4-epimerase. A previously uncharacterized gene encoding a protein that has significant identity with known UDP-galactose 4-epimerases was cloned from the yeast Schizosaccharomyces pombe, designated SpGALE as follows. The 1.1 Kb S. pombe gene encoding a predicted UDP galactose-4-epimerase (GenBank NC_{—}003423), referred to as SpGALE, was PCR amplified from S. pombe (ATCC24843) genomic DNA using primers PCR primers GALE2-L (5′-ATGACTGGTGTTCATGAAGGG-3′ (SEQ ID NO:9)) and GALE2-R (5′-TTACTTATA TGTCTTGGTATG-3′ ((SEQ ID NO:10)). The PCR amplified product was cloned into pCR2.1 (Invitrogen, Carlsbad, Calif.) and sequenced. Sequencing revealed the presence of an intron (175 bp) at the +66 position. To eliminate the intron, upstream PCR primer GD1 (5′-GCGGCCGCATGA CTGGTGTTCA TGAAGGGACT GTGTTGGTTA CTGGCGGCGC TGGTTATATA GGTTCTCATA CGTGCGTTGT TTTGTTAGAA AA-3′ ((SEQ ID NO:11)) was designed, which has a NotI site, 66 bases upstream of the intron, followed by 20 bases preceding the intron and downstream PCR primer GD2 (5′-TTAATTAATT ACTTATATGT CTTGGTATG-3′ ((SEQ ID NO:12)), which has a PacI site. Primers GD1 and GD2 were used to amplify the SpGALE intronless gene from the pCR2.1 subclone and the product cloned again into pCR2.1 and sequenced. SpGALE was then subcloned between the NotI and PacI sites into plasmids pRCD402 and pRCD403 to create plasmids pRCD406 (P_{OCH1}-SpGALE-CYC1TT) and pRCD407 (P_{SEC4}-SpGALE-CYC1TT), respectively. These plasmids have been described previously in described in U.S. Published Application No. 20060040353. The nucleotide sequence encoding SpGALE without intron is shown in SEQ ID NO:35 and the amino acid sequence shown in SEQ ID NO:36.

The human UDP galactose-4-epimerase (hGalE) has the amino acid sequence shown in SEQ ID NO:48, which is encoded by the nucleotide sequence shown in SEQ ID NO:47. The hGalE can be used in place of the SpGALE.

Construction of a double GalT/galactose-4-epimerase construct. Plasmid pXB53, containing the ScMNN2-hGalTIβ43 fusion gene, was linearized with XhoI and made blunt with T4 DNA polymerase. The P_{PpOCH1}SpGALE-CYC1TT cassette was then removed from plasmid pRCD406 with XhoI and SphI, the ends made blunt with T4 DNA polymerase, and the fragment inserted into the pXB53 plasmid above to create plasmid pRCD425. This plasmid was linearized with XbaI and transformed into strain YSH44 to generate strain RDP52, which has been previously described in described in U.S. Published Application No. 20060040353. N-glycans on purified K3 isolated from several of the transformants were analyzed by MALDI-TOF MS. As shown in FIG. 3D, a significant proportion of the N-glycans were found to have acquired a mass consistent with the addition of either two (about 20% G2: Gal₂GlcNAc₂Man₃GlcNAc₂) or a single galactose moiety (about 40% G1: Gal₁GlcNAc₂Man₃GlcNAc₂) onto the G0 (GlcNAc₂Man₃GlcNAc₂) substrate while the remainder of the N-glycans remained unchanged from that found in the YSH44 parent (FIG. 3B), that is G0.

Construction of a triple GalT/galactose-4-epimerase/UDP galactose transporter construct. The G418R plasmid containing P_{OCH1}-DmUGT-CYC1TT, pSH263, was linearized by digesting with Sad and making the ends blunt with T4 DNA polymerase. The P_{SEC4}-SpGALE-CYC1TT cassette was removed from plasmid pRCD407 by digesting with XhoI and SphI and making the ends blunt with T4 DNA polymerase. The blunt-ended SpGALE fragment was then inserted into the pSH263 above to create plasmid pRCD446. The P_{GAPDH}ScMNN2-hGalTIβ43-CYC1TT cassette was released from plasmid pXB53 by digesting with BglII/BamHI and the ends made blunt with T4 DNA polymerase. The blunt-ended hGalTI-53 was then inserted into the blunt EcoRI site of pRCD446 to create plasmid pRCD465, which is a triple G418^{R} plasmid containing hGalTI-53, SpGALE, and DmUGT. Plasmid pRCD465 was linearized with AgeI and transformed into strain YSH44 to generate strain RDP80, which as been described in described in U.S. Published Application No. 20060040353. N-glycans released from secreted K3 produced by the strain were analyzed by MALDI-TOF MS. The N-glycans were found to be of a mass consistent with the quantitative addition of two galactose residues to the G0 substrate to yield the human galactosylated, biantennary complex N-glycan, G2 (Gal₂GlcNAc₂Man₃GlcNAc₂) (FIG. 3E). In vitro β-galactosidase digestion of this N-glycan resulted in a mass decrease corresponding to the removal of two galactose residues yielding G0 (GlcNAc₂Man₃GlcNAc₂) (FIG. 3F).

In addition, in vitro treatment of purified K3 from strain RDP80 with rat et-2,6-N-sialyltransferase in the presence of CMP-Sialic acid resulted in nearly uniform conversion to NANA₂Gal₂GlcNAc₂Man₃GlcNAc₂ (FIG. 3G). These results indicate that highly efficient extension of complex N-glycans produced in P. pastoris is achievable through (1) the metabolic engineering of a sufficient intracellular UDP-galactose pool, (2) the expression of an active and properly localized GalT, and (3) the translocation of UDP-galactose into the Golgi apparatus by an active UDP-galactose transporter. However, the efficiency of galactose transfer was improved by further including the enzymes of the Leloir pathway into the host cell as shown in Example 2.

Strains and Media. E. coli strains TOP10 or DH5α were used for recombinant DNA work, P. pastoris strain YSH44 (Hamilton et al., Science 301: 1244-1246 (2003)), derived from strain JC308 (J. Cregg, Claremont, Calif.) was used for generation of various yeast strains. Transformation of yeast strains was performed by electroporation as previously reported (Cregg, et al., Mol. Biotechnol. 16: 23-52 (2000)). Protein expression was carried out at room temperature in a 96-well plate format (except for bioreactor experiments) with buffered glycerol-complex medium (BMGY) consisting of 1% yeast extract, 2% peptone, 100 mM potassium phosphate buffer, pH 6.0, 1.34% yeast nitrogen base, 4×10⁻⁵% biotin, and 1% glycerol as a growth medium; and buffered methanol-complex medium (BMMY) consisting of 1% methanol instead of glycerol in BMGY as an induction medium. YPD is 1% yeast extract, 2% peptone, 2% dextrose and 2% agar.

Restriction and modification enzymes were from New England BioLabs (Beverly, Mass.). Oligonucleotides were obtained from Integrated DNA Technologies (Coralville, Iowa). The β-Galactosidase enzyme was obtained from QA bio (San Mateo, Calif.). Ninety-six-well lysate-clearing plates were from Promega (Madison, Wis.). Protein-binding 96-well plates were from Millipore (Bedford, Mass.). Salts and buffering agents were from Sigma (St. Louis, Mo.). MALDI matrices were from. Aldrich (Milwaukee, Wis.).

Protein purification and N-glycan analysis. Purification of K3 was described previously (Choi et al., Proc. Natl. Acad. Sci. U.S.A. 100: 5022-5027 (2003)). N-glycans were released from K3 using the enzyme N-glycosidase F, obtained from New England Biolabs (Beverly, Mass.) as described previously (Choi et al., ibid.). Molecular weights of glycans were determined using a Voyager DE PRO linear MALDI-TOF Mass Spectrometer from Applied Biosystems (Foster City, Calif.) as described previously (Choi et al, ibid.).

Bioreactor Cultivations. A 500 mL baffled volumetric flask with 150 mL of BMGY media was inoculated with 1 mL of seed culture (see flask cultivations). The inoculum was grown to an OD₆₀₀ of 4-6 at 24° C. (approx 18 hours). The cells from the inoculum culture were then centrifuged and resuspended into 50 mL of fermentation media (per liter of media: CaSO₄.2H₂O 0.30 g, K₂SO₄ 6.00 g, MgSO₄.7H₂O 5.00 g, Glycerol 40.0 g, PTM₁ salts 2.0 mL, Biotin 4×10⁻³ g, H₃PO₄ (85%) 30 mL, PTM₁ salts per liter: CuSO₄.H₂O 6.00 g, NaI 0.08 g, MnSO₄.7H₂O 3.00 g, NaMoO₄.2H₂O 0.20 g, H₃BO₃ 0.02 g, CoCl₂.6H₂O 0.50 g, ZnCl₂ 20.0 g, FeSO₄.7H₂O 65.0 g, Biotin 0.20 g, H₂SO₄ (98%) 5.00 mL).

Fermentations were conducted in three-liter dished bottom (1.5 liter initial charge volume) Applikon bioreactors. The fermenters were run in a fed-batch mode at a temperature of 24° C., and the pH was controlled at 4.5±0.1 using 30% ammonium hydroxide. The dissolved oxygen was maintained above 40% relative to saturation with air at 1 atm by adjusting agitation rate (450-900 rpm) and pure oxygen supply. The air flow rate was maintained at 1 vvm. When the initial glycerol (40 g/L) in the batch phase is depleted, which is indicated by an increase of DO, a 50% glycerol solution containing 12 ml/L of PTM₁ salts was fed at a feed rate of 12 mL/L/h until the desired biomass concentration was reached. After a half an hour starvation phase, the methanol feed (100% methanol with 12 mL/L PTM₁) is initiated. The methanol feed rate is used to control the methanol concentration in the fermenter between 0.2 and 0.5%. The methanol concentration is measured online using a TGS gas sensor (TGS822 from Figaro Engineering Inc.) located in the offgas from the fermenter. The fermenters were sampled every eight hours and analyzed for biomass (OD₆₀₀, wet cell weight and cell counts), residual carbon source level (glycerol and methanol by HPLC using Aminex 87H) and extracellular protein content (by SDS page, and Bio-Rad protein assay).

In vitro β-galactosidase digest. N-glycans from RDP80 were incubated with β1,4-galactosidase (QA bio, San Mateo, Calif.) in 50 mM NH₄HCO₃, pH6.0 at 37° C. for 16-20 hours.

In vitro sialic acid transfer. K3 purified from strain RDP80 was used as the substrate for sialic acid transfer. Of this protein, 200 μg was incubated with 50 pg CMP-sialic acid and 15 mU rat recombinant α-(2,6)-(N)-sialyltransferase from EMD Biosciences (San Diego, Calif., formerly Calbiochem) in 50 mM NH₄HCO₃, pH6.0 at 37° C. for 16-20 hours. N-glycan was then released by PNGaseF digest and detected by MALDI-TOF MS.

### Example 2

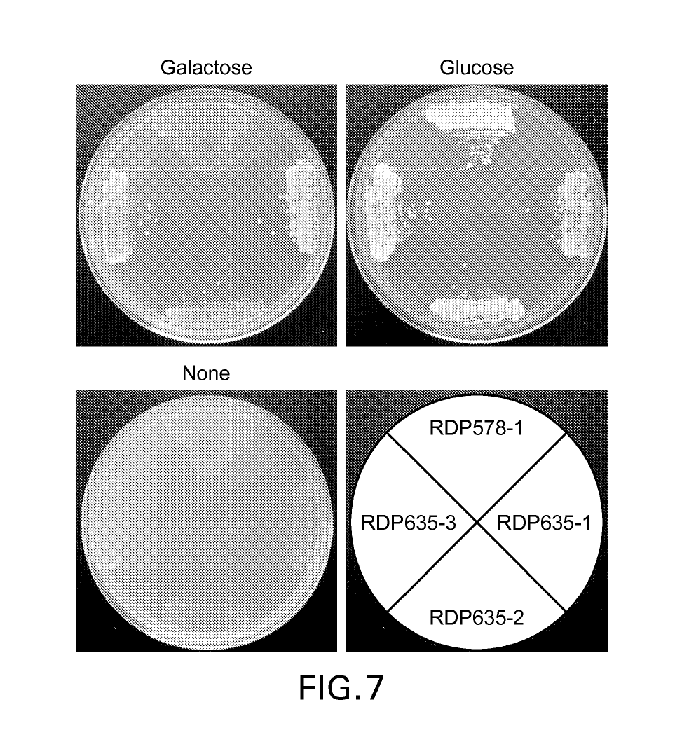

The enzyme UDP-galactose 4-epimerase catalyzes the 3^{rd} step of the Leloir pathway (FIG. 4). As shown in Example 1, heterologous expression of the gene encoding this enzyme in a glycoengineered strain of P. pastoris resulted in the generation of an intracellular pool of UDP-galactose as evidenced by the dramatic increase in galactose transfer in strains expressing this heterologous gene. However, as also shown, addition of this enzyme alone did not confer upon P. pastoris strains the ability to grow on galactose as a sole carbon source (See FIG. 7, strain RDP578-1). Therefore, the remainder of the Leloir pathway in S. cerevisiae was introduced into various strains of Example 1. Thus, in this example, a Pichia pastoris host cell capable of using galactose as a sole carbon source was constructed. The methods herein can be used to make recombinant host cells of other species that are normally incapable of using galactose as a carbon source into a recombinant host cell that is capable of using galactose as a sole carbon source.

Cloning of S. cerevisiae GAL1. The S. cerevisiae gene encoding the galactokinase (GenBank NP_{—}009576) referred to as ScGAL1 was PCR amplified from S. cerevisiae genomic DNA (Strain W303, standard smash and grab genomic DNA preparation) using PCR primers PB158 (5′-TTAGCGGCCGCAGGAATGACTAAATCTCATTCA-3′ (SEQ ID NO:13)) and PB159 (5′-AACTTAATTAAGCTTATAATTCATATAGACAGC-3′ (SEQ ID NO:14)) and the PCR amplified DNA fragment was cloned into pCR2.1 (Invitrogen, Carlsbad, Calif.) and sequenced. The resulting plasmid was named pRCD917. The DNA fragment encoding the galactokinase was released from the plasmid with NotI and PacI and the DNA fragment subcloned into plasmid pGLY894 downstream of the P. pastoris HHT1 strong constitutive promoter between the NotI and PacI sites to create plasmid pGLY939. The galactokinase has the amino acid sequence shown in SEQ ID NO:40 and is encoded by the nucleotide sequence shown in SEQ ID NO:39.

Cloning of S. cerevisiae GAL2. The S. cerevisiae gene encoding the galactose permease (GenBank NP_{—}013182) referred to as ScGAL2 was PCR amplified from S. cerevisiae genomic DNA (Strain W303, standard “smash and grab” genomic DNA preparation) using PCR primers PB156 (5′-TTAGCGGCCGC-3′ (SEQ ID NO:15)) and PB157 (5′-AACTTAATTAA-3′ (SEQ ID NO:16)) and the PCR amplified DNA fragment was subcloned into pCR2.1 (Invitrogen, Carlsbad, Calif.) and sequenced. The resulting plasmid was named pPB290. The DNA fragment encoding the galactose permease was released from the plasmid with NotI and PacI and the DNA fragment subcloned into plasmid pJN664 downstream of the PpPMA1 promoter between the NotI and PacI sites to create plasmid pPB292. The galactose permease has the amino acid sequence shown in SEQ ID NO:44 and is encoded by the nucleotide sequence shown in SEQ ID NO:43.

Cloning of S. cerevisiae GAL7. The S. cerevisiae gene encoding the galactose-1-phosphate uridyl transferase (GenBank NP_{—}009574) referred to as ScGAL7 was PCR amplified from S. cerevisiae genomic DNA (Strain W303, standard smash and grab genomic DNA preparation) using PCR primers PB160 (5′-TTAGCGGCCG CAGGAATGAC TGCTGAAGAA TT-3′ (SEQ ID NO:17)) and PB161 (5′-AACTTAATTA AGCTTACAGT CTTTGTAGAT AATC-3′ (SEQ ID NO:18) and the PCR amplified DNA fragment was cloned into pCR2.1 (Invitrogen, Carlsbad, Calif.) and sequenced. The resulting plasmid was named pRCD918. The DNA fragment encoding the galactose-1-phosphate uridyl transferase was released from the plasmid with NotI and PacI and the DNA fragment subcloned into plasmid pGLY143 downstream of the PpPMA1 strong constitutive promoter at NotI/PacI to create plasmid pGLY940. Separately, the NotI and PacI sites were also used to subclone this ORF into plasmid pRCD830 downstream of the P. pastoris TEF1 strong constitutive promoter at NotI/PacI to create plasmid pRCD929. The galactose-1-phosphate uridyl transferase has the amino acid sequence shown in SEQ ID NO:42 and is encoded by the nucleotide sequence shown in SEQ ID NO:41.

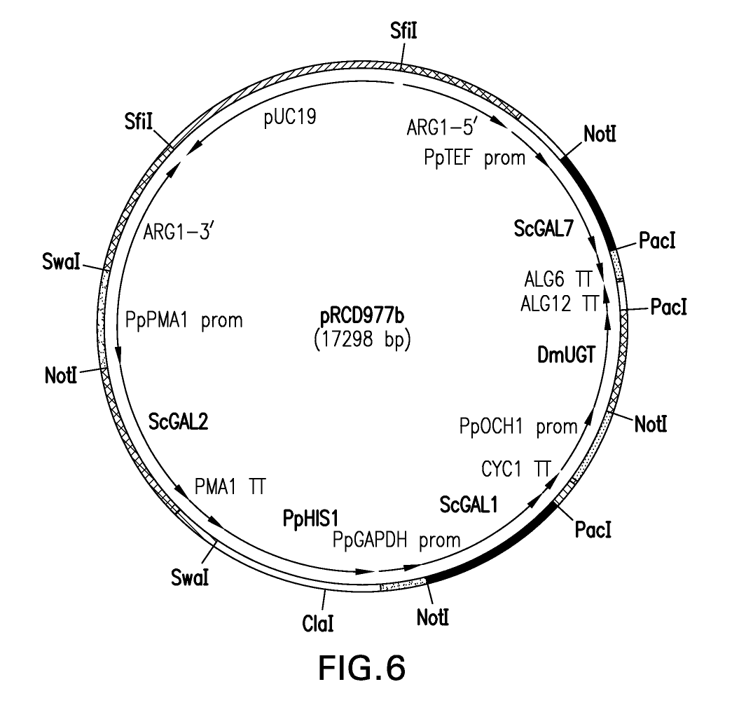

Construction of a triple ScGAL1/ScGAL7/ScGAL2 construct. The ScGAL1 open reading frame from pGLY917 was subcloned into pJN702, a P. pastoris his1 knock-out vector with the P. pastoris ARG1 selectable marker (his1::ARG1, see U.S. Pat. No. 7,479,389), and also containing a P_{GAPDH}-promoter cassette and this new vector containing the P_{GAPDH}-ScGAL1 fusion was named pRCD928. The P_{TEF1}-ScGAL7 cassette from pGLY929 was subcloned into pGLY928 and the new vector was named pGLY946a. Next, the P_{OCH1}-DmUGT (Golgi UDP-galactose transporter) cassette from pRCD634 was subcloned into pGLY946a to create pGLY956b. Finally, the P_{GAPDH}-ScGAL2 cassette from pPB292 was subcloned into pRCD956b to produce plasmid pRCD977b (See FIG. 6). Plasmid pRCD977b contains DmUGT, ScGAL1, ScGAL7, and ScGAL2 expression cassettes along with the ARG1 dominant selectable marker cassette.

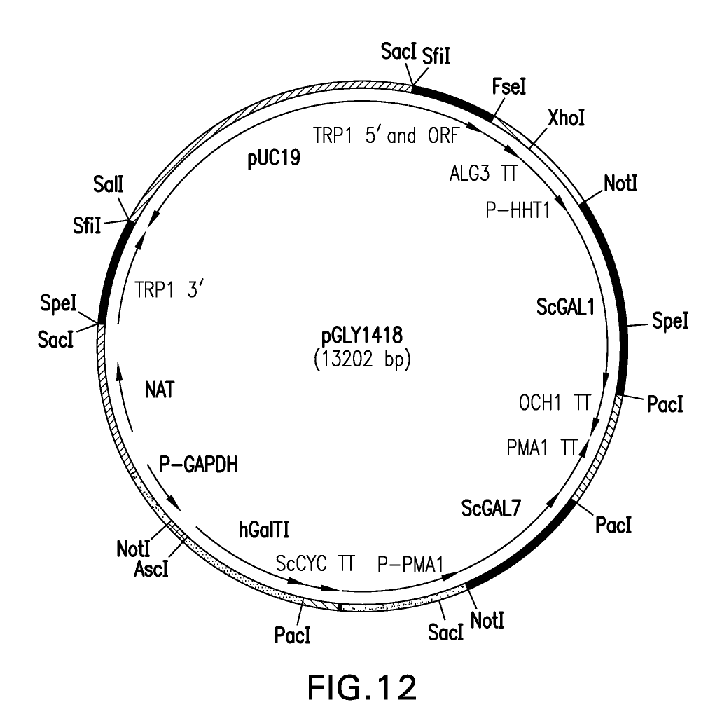

Construction of a double ScGAL1/ScGAL7 construct. The P_{TEF1}-ScGAL1 cassette from pGLY939 was subcloned into pGLY941, a knock-in vector with the P. pastoris ARG1 selectable marker, TRP1 locus knock-in region, and also containing a P_{GAPDH}-hGalTIβ cassette and this new vector was named pGLY952. The P_{PMA1}-ScGAL7 cassette from pGLY940 was subcloned into pGLY952 and the new vector was named pGLY955. Finally, the Nourseothricin resistance cassette (NAT^{R}) was subcloned from pGLY597 (originally from pAG25 from EROSCARF, Scientific Research and Development GmbH, Daimlerstrasse 13a, D-61352 Bad Homburg, Germany, See Goldstein et al., 1999, Yeast 15: 1541) into pGLY952 to produce plasmid pGLY1418 (See FIG. 12), which contains hGalTIβ, ScGAL1, and ScGAL7 expression cassettes along with the NAT^{R} dominant selectable marker cassette.

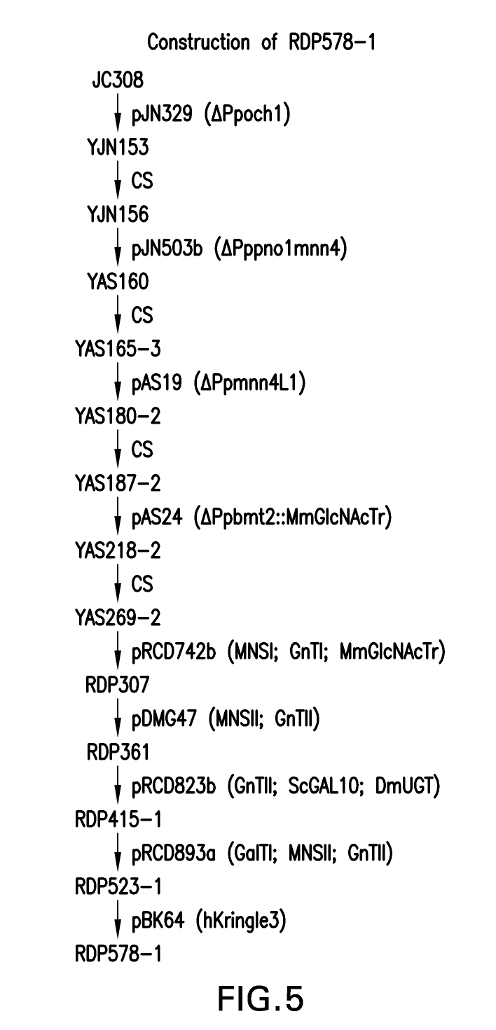

The single integration plasmid harboring all three genes, pRCD977b (FIG. 6), was transformed into the P. pastoris strain RDP578-1 to produce strains RDP635-1, -2, and -3. Strain RDP578-1 already contained the heterologous genes and gene knockouts for producing human N-glycan containing terminal β-1,4-galactose residues (See FIG. 5 and Example 3 for construction. Strain RDP578-1 also includes an expression cassette encoding the Saccharomyces cerevisiae UDP-Galactose 4-epimerase encoding gene, ScGAL10, and expresses the test protein human kringle 3. The resulting strains RDP635-1, -2, and -3 have two copies of the DmUGT galactose transporter.

The parental strain, RDP578-1, and the transformants with the ScGAL1, ScGAL2, ScGAL7, and ScGAL10 genes (RDP635-1, -2, and -3) were grown on minimal medium containing glucose, galactose, or no carbon source for five days and then photographed. Interestingly, despite having the ability to secrete proteins with galactose-terminated N-glycans, RDP578-1 displayed no ability to assimilate galactose, while growing normally on glucose, as would be expected for wild-type P. pastoris. However, the transformants expressing the ScGAL1, ScGAL2, and ScGAL7 genes were capable of assimilating galactose as shown in FIG. 7. As expected, minimal growth was observed on the plates lacking a carbon source. These results indicate that a recombinant P. pastoris can be constructed that can assimilate galactose as a carbon source when reconstituted with the basic structural (but not regulatory) elements of the Leloir galactose assimilation pathway.

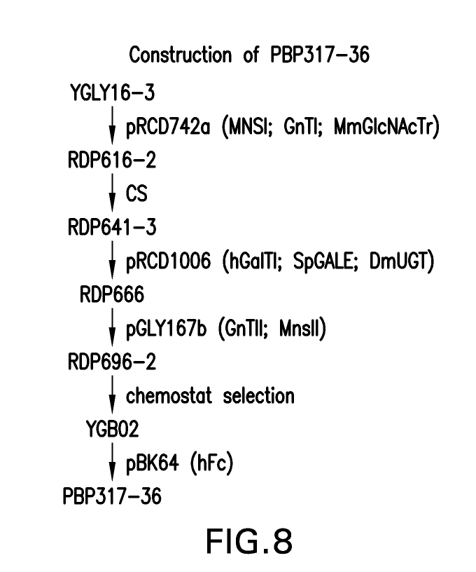

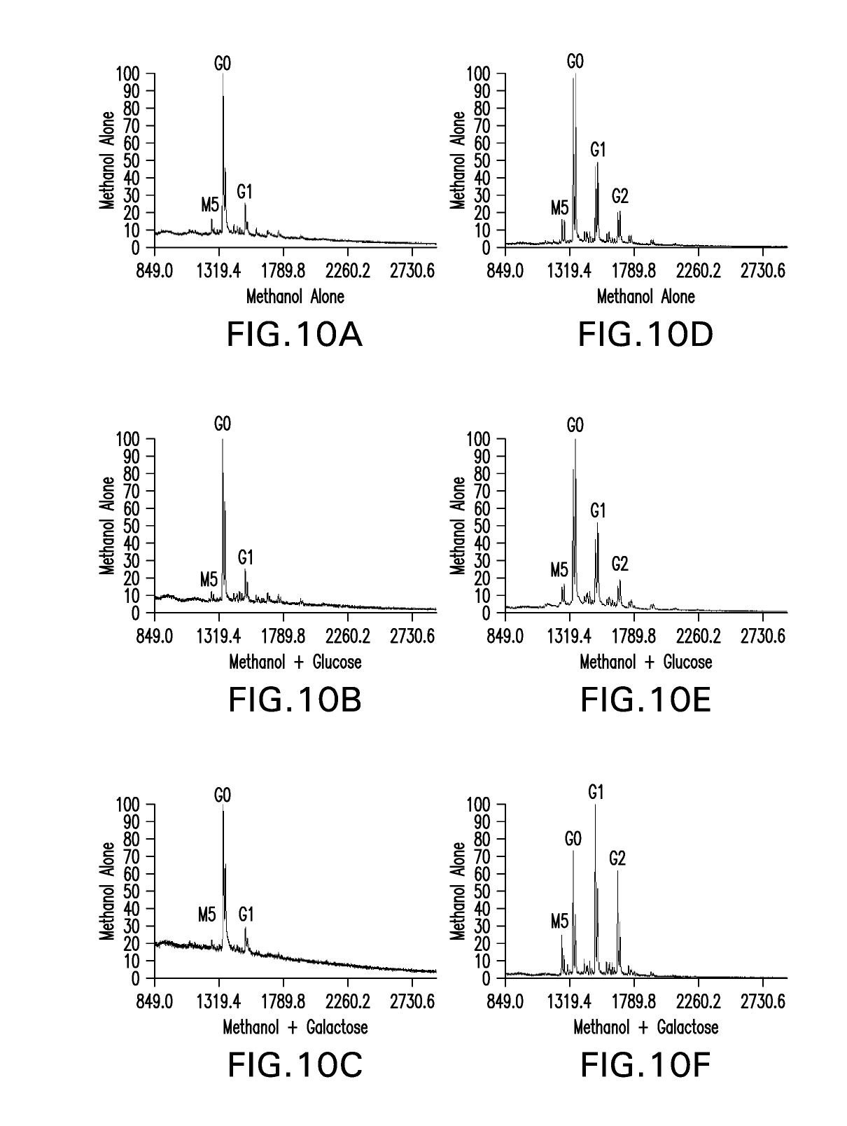

Determination of N-glycans at Asn residue 297 of Fc expressed in glycoengineered P. pastoris. The Fc portion of human IgGs contains a single N-glycan site per heavy chain dimer (Asn₂₉₇, Kabat numbering) that typically contains an N-glycan profile distinct from that of other secreted human proteins. Generally, naturally occurring human antibodies contain N-glycans with terminal GlcNAc and an amount of terminal galactose that can differ based on various factors and rarely contain a significant amount of terminal sialic acid. After demonstrating a high level of terminal β-1,4-galactose to N-glycans, we sought to determine the profile of N-glycans that are observed on antibodies produced in such a glycoengineered yeast strain. Therefore, a P. pastoris strain that had been genetically engineered to produce N-glycans with terminal galactose (YGB02; See FIG. 8 and Example 4) was transformed with a plasmid (pBK138) encoding the Fc domain or C-terminal half of the human Immunoglobulin G1 (IgG1) heavy chain under control of the AOX1 promoter. A selected positive clone identified by PCR was named PBP317-36 (FIG. 8 and Example 4). This strain was grown in a shake flask and induced with methanol as a sole carbon source. The supernatant was harvested by centrifugation and was subjected to purification by protein A affinity chromatography. Purified protein was separated on SDS-PAGE and coomassie stained. A labeled band of the expected size was observed. The purified protein was then subjected to PNGase digestion and the released N-glycans analyzed by MALDI-TOF MS. The resulting N-glycans (FIG. 10A) revealed a predominant mass consistent with a complex human core structure with terminal GlcNAc (G0: GlcNAc₂Man₃GlcNAc₂), a lesser species with a single terminal galactose (G1: GalGlcNAc₂Man₃GlcNAc₂), and a minor species where both arms of a complex species are capped with galactose (G2: Gal₂GlcNAc₂Man₃GlcNAc₂). The masses of these species differed predictably from the canonical values reported in the literature due to the lack of a single fucose residue. Glycoengineered yeast strains do not contain an endogenous fucosyltransferase and therefore lack the inherent ability to add a fucose to the core human N-glycan structure. Another minor species consistent with Man₅GlcNAc₂ was also observed.

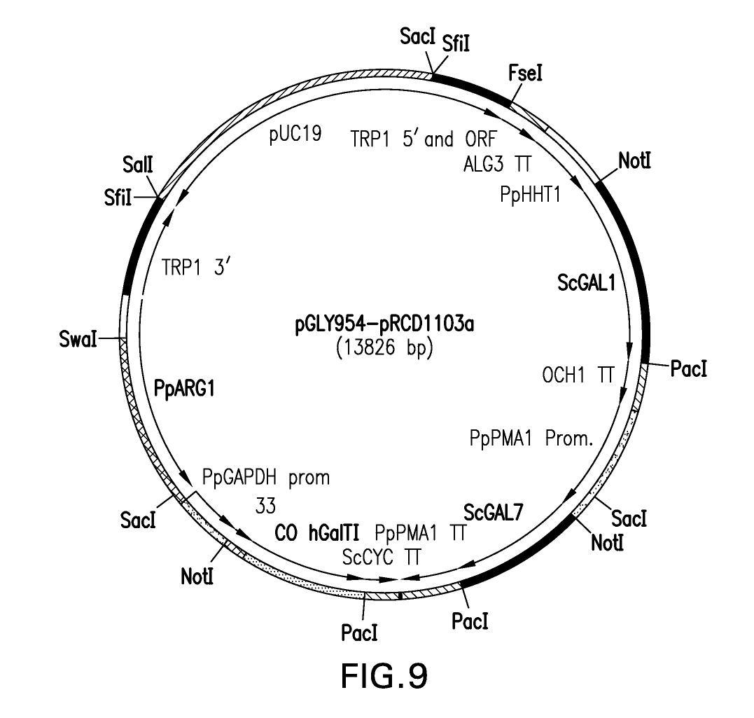

Strain PBP317-36 above which expressed the glycosylation activities required to assemble human-like N-glycans with terminal galactose and which also expressed SpGALE (UDP-galactose 4-epimerase) was then genetically engineered to be able to use exogenous galactose as a carbon source and to control N-glycosylation in a metabolically engineered manner. An integration plasmid expressing both ScGAL1 and ScGAL7 under the control of a constitutive promoter, pGLY954, was constructed (FIG. 9) and transformed into the P. pastoris strain PBP317-36. Plasmid pGLY954 conferred upon strain PBP317-36 (which already contained the SpGALE UDP-galactose epimerase, FIG. 8) the ability to grow on galactose as a sole carbon source. This gal⁺ strain was named RDP783. Because the cells could use galactose as a carbon source even though we had not introduced the galactose permease into the cell, we concluded that general hexose transporters endogenous to P. pastoris are able to transport galactose sufficiently across the cell membrane.

P. pastoris strain PBP317-36 and RDP783 both harbor an integrated plasmid construct encoding the human Fc domain as a secreted reporter protein under control of the methanol-inducible AOX1 promoter. Strains PBP317-36 and RDP783 were grown in shake flasks in standard media containing glycerol and induced in the presence of either methanol as a sole carbon source or with methanol combined with glucose or galactose at different concentrations. Harvested supernatant protein was affinity purified by protein A, subjected to PNGase digestion, and analyzed by MALDI-TOF MS. N-glycans released from the human Fc from strain RDP783 yielded a similar N-glycan to the profile observed with PBP317-36 upon methanol induction alone or in the presence of glucose or mannose, with the predominant glycoform G0 (GlcNAc₂Man₃GlcNAc₂) (FIG. 10). However upon exogenous galactose feed, strain RDP783 (but not the parent strain PBP317-36) yielded a dose-dependent increase in galactose-containing N-glycans on the human Fc, with a shift in the predominant glycoform now to G1 (GalGlcNAc₂Man₃GlcNAc₂) and a concomitant increase in the fully β-1,4-galactose capped glycoform G2 (Gal₂GlcNAc₂Man₃GlcNAc₂) (FIG. 10).

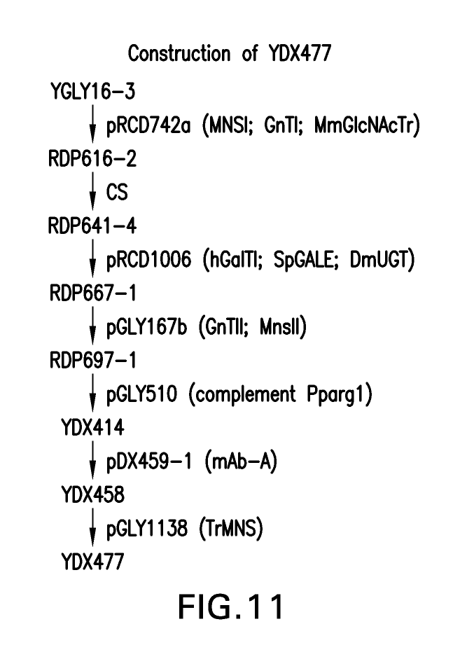

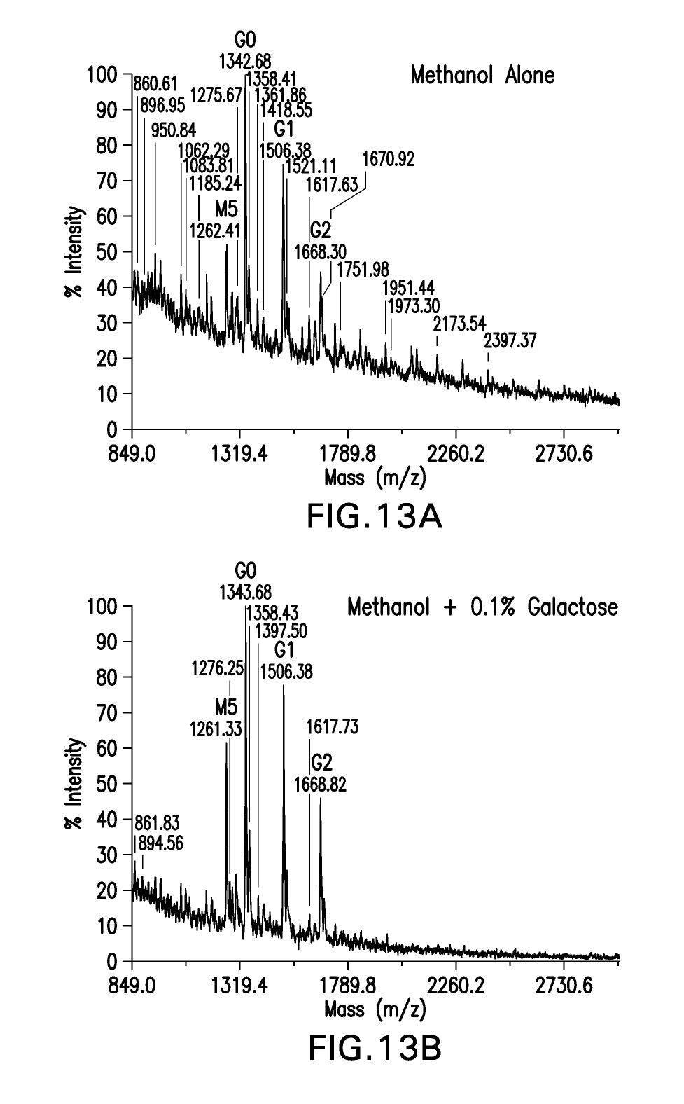

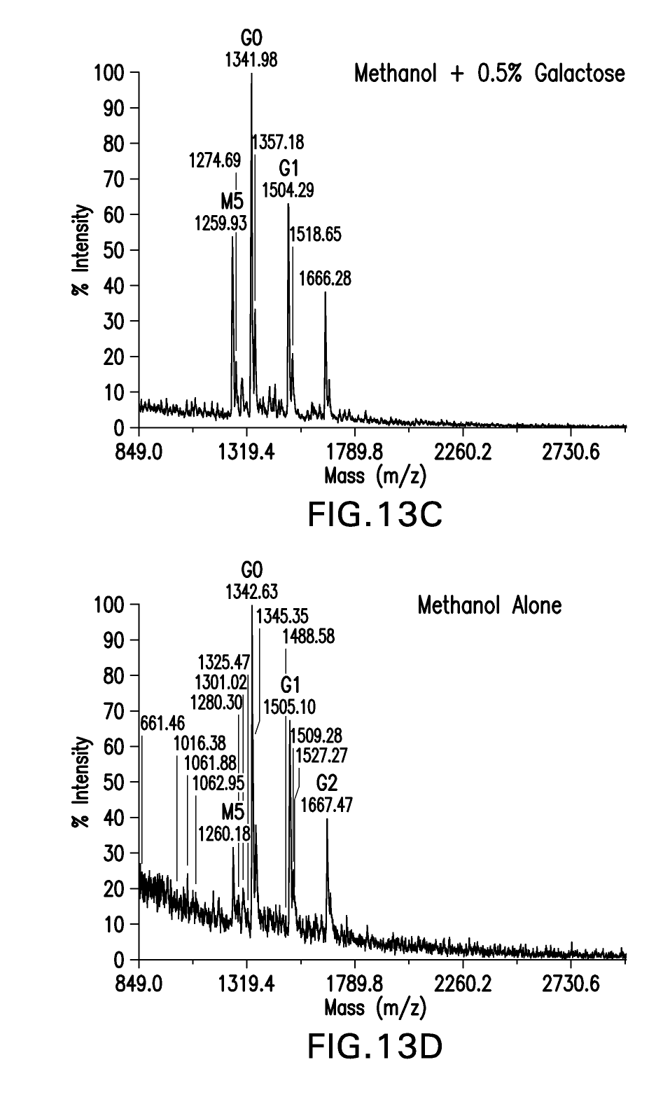

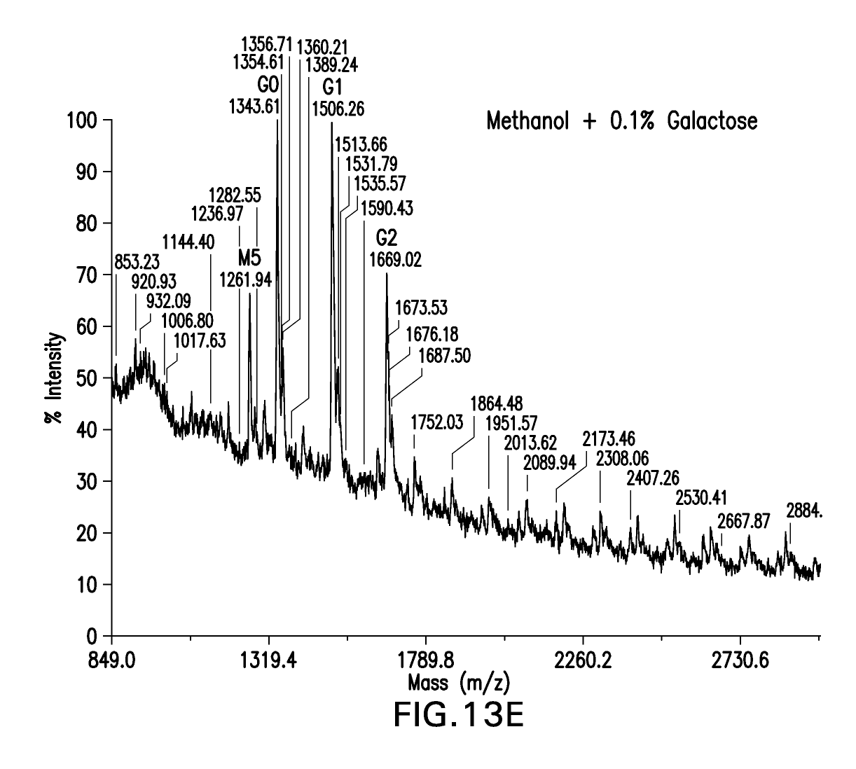

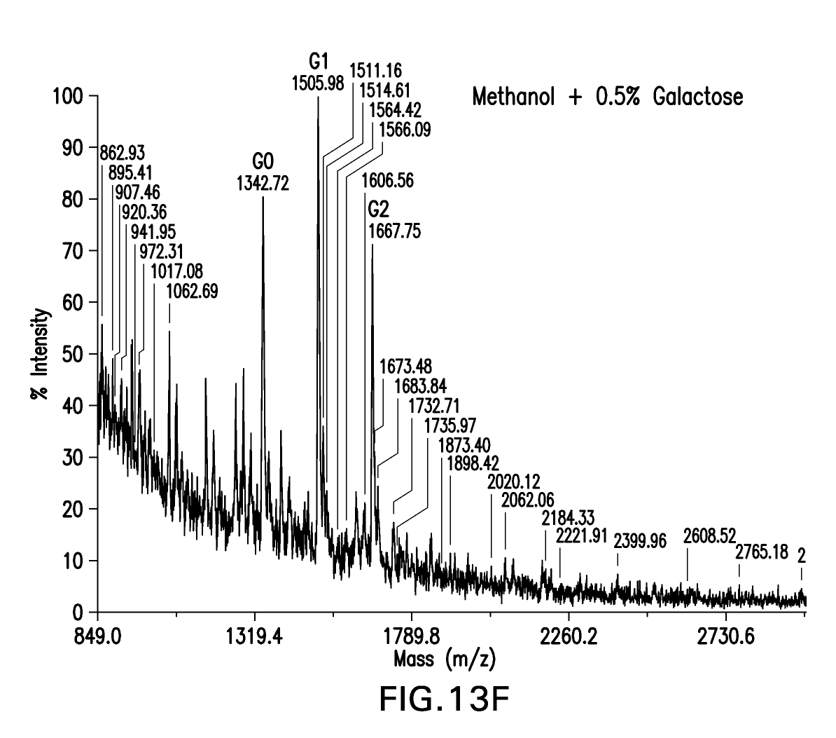

Finally, to demonstrate that the ability to control glycosylation using exogenous galactose observed with the human Fc could be applied to a full-length monoclonal antibody, a glycoengineered yeast strain was generated, YDX477 (FIG. 11, Example 5), that expresses an anti-Her2 monoclonal antibody. This strain was also engineered to transfer human N-glycans of the form G2 (Gal₂GlcNAc₂Man₃GlcNAc₂) on secreted glycoproteins. Release of N-glycans after expression of mAb-A revealed an N-glycan pattern (FIG. 13A) consisting of a predominant peak consistent with G0 (GlcNAc₂Man₃GlcNAc₂), with a less predominant peak consistent with G1 (GalGlcNAc₂Man₃GlcNAc₂), as well as minor peaks of G2 (Gal₂GlcNAc₂Man₃GlcNAc₂) and M5 (Man₅GlcNAc₂). This data is similar with what was observed for the truncated Fc portion of human IgG1 and was expected because both are N-glycosylated at the same residue (Asn-297). An integration plasmid harboring ScGAL1 and ScGAL7, pGLY1418, was constructed (FIG. 12) and transformed into the P. pastoris strain YDX477 to make strain RDP968-1. This plasmid conferred upon strain YDX477 (which already contains the SpGALE UDP-galactose epimerase, FIG. 11) the ability to grow on galactose as a sole carbon source.

Strains YDX477 and RDP968-1 were grown in shake flasks in standard media containing glycerol and induced in the presence of either methanol as a sole carbon source or with methanol combined with galactose at different concentrations. Harvested supernatant protein was affinity purified by protein A, subjected to PNGase digestion, and analyzed by MALDI-TOF MS. Both strains yielded N-glycans similar to the profile observed previously with PBP317-36 upon methanol induction alone or in the presence of glucose or mannose, with the predominant glycoform G0 (GlcNAc₂Man₃GlcNAc₂) (FIG. 10A vs. FIGS. 13A and 13D). However upon exogenous galactose feed, strain RDP968-1 (but not the parent strain YDX477) yielded a dose-dependent increase in galactose-containing N-glycans on the antibody, with a shift in the predominant glycoform now to G1 (GalGlcNAc₂Man₃GlcNAc₂) and a concomitant increase in the fully β-1,4-galactose capped glycoform G2 (Gal₂GlcNAc₂Man₃GlcNAc₂) (FIGS. 13E and F).

### Example 3

Construction of strain RDP578-1 is shown in FIG. 5 and involved the following steps. Strain JC308 was the starting strain. This strain has been described in Choi et al., Proc. Natl. Acad. Sci. USA 100: 5022-5027 (2003) but briefly, the strain is ura3, ade1, arg4, his4. This strain was rendered deficient in alpha-1,6 mannosyltransferase activity by disrupting the OCH1 gene using plasmid pJN329 and following the procedure described in Choi et al. (ibid.) and in U.S. Pat. No. 7,449,308 to produce strain YJN153. Plasmid pJN329 carries the PpURA3 dominant selection marker, after counterselecting for ura− activity, resulting strain YJN156 was rendered deficient in phosphomannosyltransferase activity by disrupting the PNO1, MMN4A, and MNN4B genes using plasmid vectors pJN503b and pAS19 following the procedure described in U.S. Pat. No. 7,259,007 to produce strain YAS180-2. The secretory pathway targeting leader peptides comprising the fusion proteins herein localize the catalytic domain it is fused to the ER, Golgi, or the trans Golgi network.

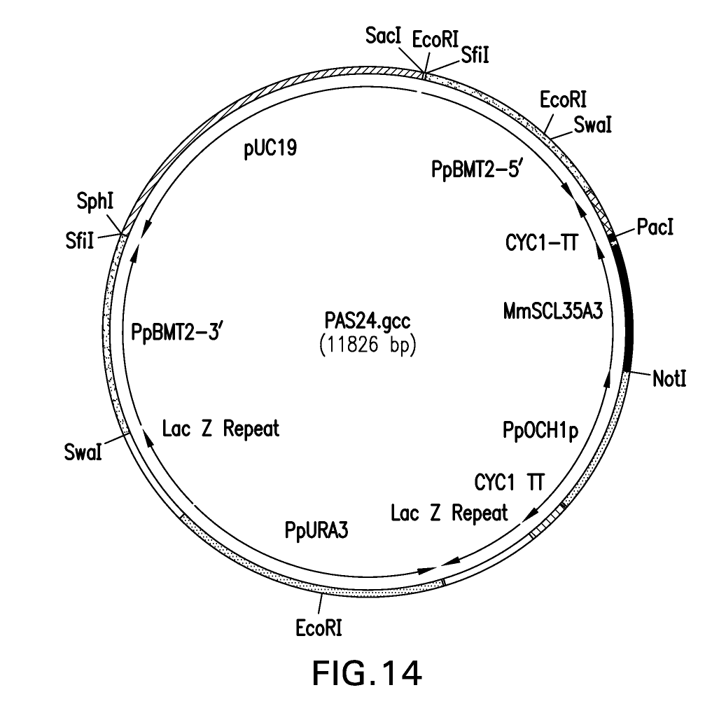

After counterselecting for ura− activity, resulting strain YAS187-2 was rendered deficient in beta-mannosyltransferase activity generally as described in U.S. Pat. No. 7,465,577 using plasmid pAS24 (See FIG. 14) to make strain YAS218-2. Plasmid pAS24 is a P. pastoris BMT2 knock-out plasmid that contains the PpURA3 selectable marker and contains an expression cassette encoding the full length mouse Golgi UDP-GlcNAc Transporter (MmSLC35A3) downstream of the PpOCH1 promoter. MmSLC35A3 has the amino acid sequence shown in SEQ ID NO:34 which is encoded by the nucleotide sequence shown in SEQ ID NO:33. 5′ and 3′ BMT2 flanking sequences for removing beta-mannosyltransferase activity attributed to bmt2p can be obtained as shown in U.S. Pat. No. 7,465,577. After counterselecting strain YAS218-2 for ura− activity, resulting strain YAS269-2 is ura− and has the mouse Golgi UDP-GlcNAc Transporter inserted into the BMT2 gene.

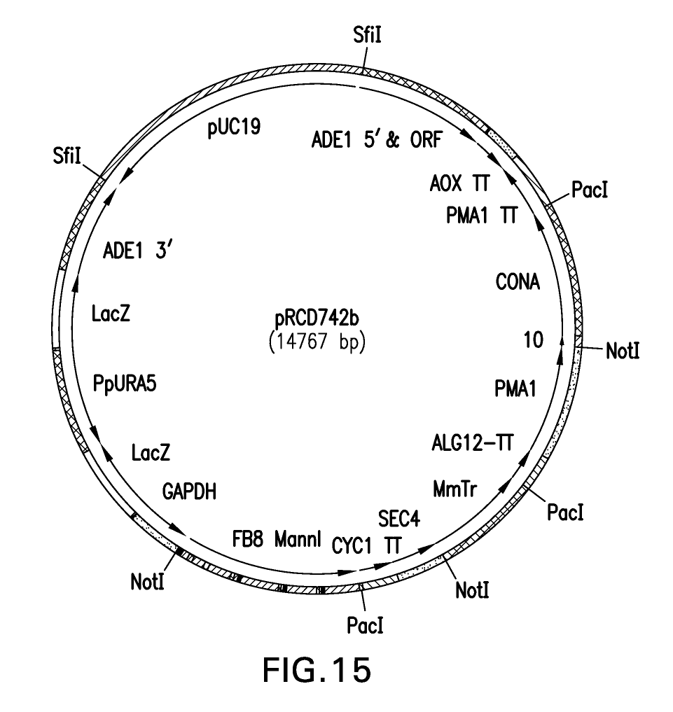

Strain YAS269-2 was then transformed with plasmid pRCD742b (See FIG. 15), which comprises expression cassettes encoding a chimeric mouse alpha-1,2-mannosyltransferase I (FB8 MannI), a chimeric human GlcNAc Transferase I (CONA10), and the full-length gene encoding the Mouse Golgi UDP-GlcNAc transporter (MmSLC35A3) and targets the plasmid to the ADE1 locus (See PCT/US2008/13719). Plasmid pRCD742b is a Knock-In Knock-Out (KINKO) plasmid, which has been described in WO2007/136865 and WO2007136752. The plasmid integrates into the P. pastoris ADE1 gene without deleting the open reading frame encoding the Ade1p. The plasmid also contains the PpURA5 selectable marker. The expression cassette encoding a secretory pathway targeted fusion protein (FB8 MannI) comprises a ScSec12 leader peptide (the first 103 amino acids of SeSec12 (8): SEQ ID NO:32) fused to the N-terminus of the mouse alpha-1,2-mannosyltransferase I catalytic domain (FB MannI: SEQ ID NO:54) under the control of the PpGAPDH promoter. The expression cassette encoding the secretory pathway targeted fusion protein CONA10 comprises a PpSec12 leader peptide (the first 29 amino acids of PpSec12 (10): SEQ ID NO:28) fused to the N-terminus of the human GlcNAc Transferase I (GnT I) catalytic domain (SEQ ID NO:52) under the control of the PpPMA1 promoter. The plasmid further included an expression cassette encoding the full-length mouse Golgi UDP-GlcNAc transporter (MmSLC35A3) under the control of the PpSEC4 promoter. Transfection of plasmid pRCD742b into strain YAS269-2 resulted in strain RDP307. This strain is capable of making glycoproteins that have GlcNAcMan₅GlcNAc₂ N-glycans. SEQ ID NOs:53 and 51 are the nucleotide sequences encoding the mouse alpha-1,2-mannosyltransferase I and human GlcNAc Transferase I (GnT I) catalytic domains, respectively. The nucleotide sequence encoding the human GnT I was codon-optimized for expression in Pichia pastoris. SEQ ID NOs:27 and 31 are the nucleotide sequences encoding the PpSEC12 (10) and the ScSEC12 (8), respectively.

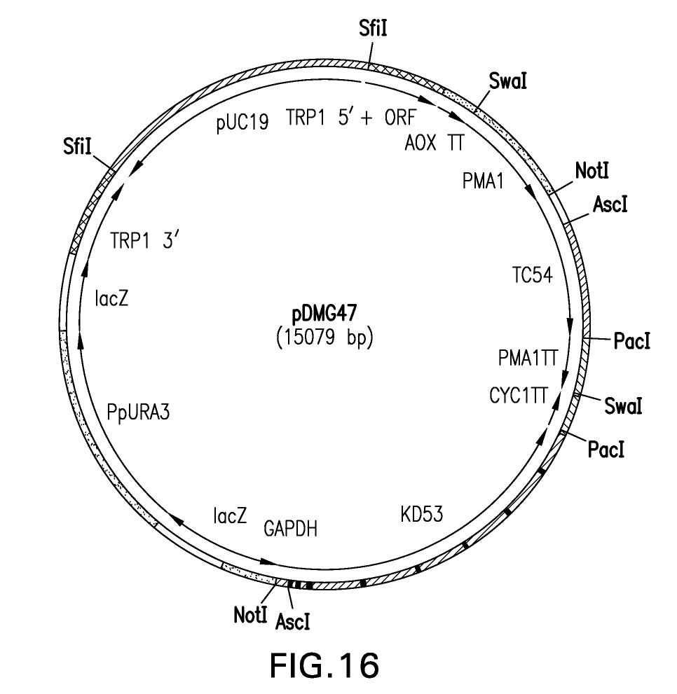

Strain RDP361 was constructed by transforming strain RDP307 with plasmid pDMG47 to produce strain RDP361. Plasmid pDMG47 (See FIG. 16) is a KINKO plasmid that integrates into the P. pastoris TRP1 locus without deleting the open reading frame encoding the Trp1p. The plasmid also contains the PpURA3 selection marker and comprises an expression cassette encoding a secretory pathway targeted fusion protein (KD53) comprising an ScMnn2 leader targeting peptide (the first 36 amino acids of ScMnn2 (53): SEQ ID NO:19) fused to the N-terminus of the catalytic domain of the Drosophila melanogaster Mannosidase II (KD: SEQ ID NO:63) under the control of the PpGAPDH promoter. The plasmid also contains an expression cassette encoding a secretory pathway targeted fusion protein (TC54) comprising an ScMnn2 leader targeting peptide (the first 97 amino acids of ScMnn2 (54): SEQ ID NO:22) fused to the N-terminus of the catalytic domain of the rat GlcNAc Transferase II (TC: SEQ ID NO:58) under the control of the PpPMA1 promoter. The nucleic acid sequence of the ScMnn2 leaders 53 and 54 are shown in SEQ ID NOs:19 and 21, respectively. The nucleic acid sequences encoding the catalytic domains of the Drosophila melanogaster mannosidase II and rat GlcNAc transferase II (GnT II) are shown in SEQ ID NOs:62 and 57, respectively.

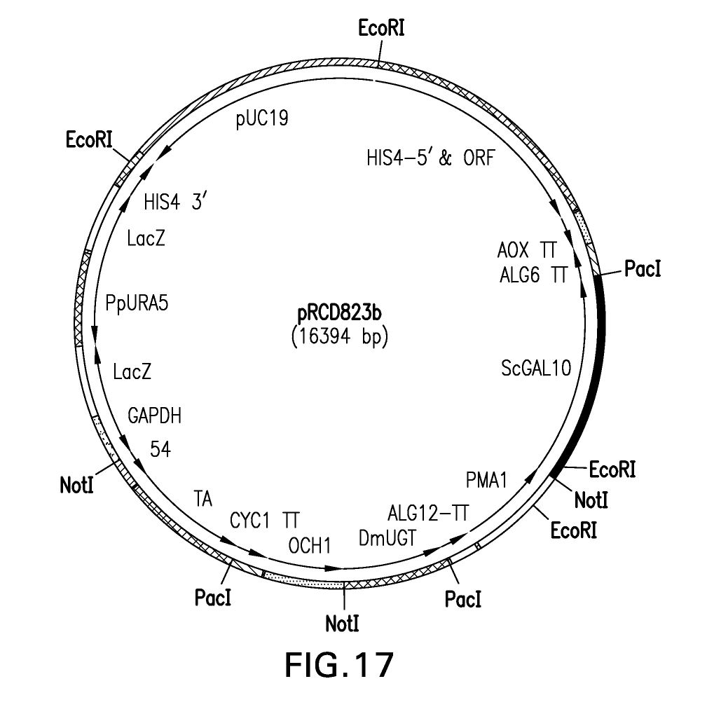

Strain RDP361 above was transformed with plasmid pRCD823b to produce strain RDP415-1. Plasmid pRCD823b (See FIG. 17) is a KINKO plasmid that integrates into the P. pastoris HIS4 locus (See U.S. Pat. No. 7,479,389) without deleting the open reading frame encoding the His4p and contains the PpURA5 selectable marker (See U.S. Pub. Application No. 20040229306) as well as an expression cassette encoding a secretory pathway targeted fusion protein (TA54) comprising the rat GlcNAc Transferase II catalytic domain (TA: SEQ ID NO:61) fused at its N-terminus to the first 97 amino acids of ScMnn2 (54) as above but under the control of the PpGAPDH promoter. The plasmid also contains expression cassettes encoding the full-length D. melanogaster Golgi UDP-galactose transporter (DmUGT) under the control of the PpOCH1 promoter and the full-length S. cerevisiae UDP-galactose 4-epimerase (ScGAL10) under the control of the PpPMA1 promoter. The ScGAL10 has the amino acid sequence shown in SEQ ID NO:46, which is encoded by the nucleotide sequence shown in SEQ ID NO:45. The nucleotide sequence of rat GlcNAc Transferase II (TA) is shown in SEQ ID NO:60.

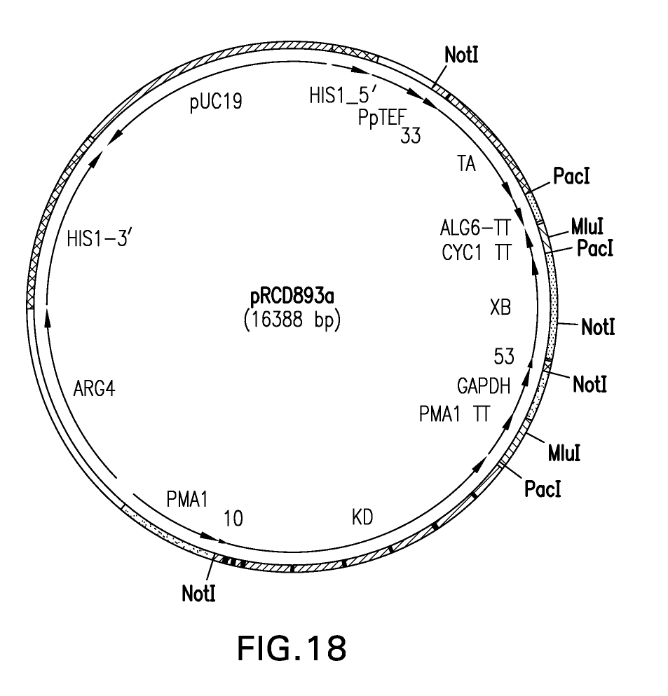

Strain RDP415-1 above was transformed with plasmid pRCD893a to produce strain RDP523-1. Plasmid pGLY893a (See FIG. 18) is a P. pastoris his1 knock-out plasmid that contains the PpARG4 selectable marker (See U.S. Pat. No. 7,479,389). The plasmid comprises an expression cassette encoding a secretory pathway targeted fusion protein (KD10) comprising a PpSEC12 leader targeting peptide (the first 29 amino acids of PpSEC12 (10): SEQ ID NO:28) fused to the N-terminus of the catalytic domain of the Drosophila melanogaster Mannosidase II (KD: SEQ ID NO:63) under the control of the PpPMA1 promoter. The plasmid also contains an expression cassette encoding a secretory pathway targeted fusion protein (TA33) comprising an ScMntIp (ScKre2p) leader targeting peptide (the first 53 amino acids of ScMntIp (ScKre2p) (33): SEQ ID NO:30) fused to the N-terminus of the catalytic domain of the rat GlcNAc Transferase II (TA: SEQ ID NO:61) under the control of the PpTEF1 promoter. The plasmid also contains an expression cassette encoding a secretory pathway targeted fusion protein (XB53) comprising the first 36 amino acids of ScMnn2p leader peptide (53) fused to the N-terminus of the catalytic domain of the human Galactosyl Transferase I (hGalTIβ43; SEQ ID NO:50). The nucleic acid sequence of the PpSEC12 and ScMNTI (ScKRE2) leaders are shown in SEQ ID NOs:27 and 29, respectively. The nucleic acid sequences encoding the catalytic domains of the Drosophila melanogaster mannosidase II, rat GlcNAc transferase II (GnT II), and human GalTI are shown in SEQ ID NOs:62, 60, and 49, respectively. This strain can make glycoproteins that have N-glycans that have terminal galactose residues. The strain encodes two copies of the Drosophila melanogaster mannosidase II catalytic domain and three copies of the rat GnT II catalytic domain.

Finally, strain RDP523-1 above was transformed with plasmid pBK64 to produce strain RDP578-1. Plasmid pBK64 encodes the human kringle3 test protein and has been described in Choi et al., Proc. Natl. Acad. Sci. USA 100: 5022-5027 (2003).

### Example 4

Construction of strain PBP317-36 is shown in FIG. 8. The starting strain was YGLY16-3. This is a ura⁻ strain with deletions of the OCH1, PNO1, MNN4A, Mnn4B, and the BMT2 genes and can be made following the process that was used in Example 3. Strain YGLY16-3 has also been disclosed in WO2007136752.

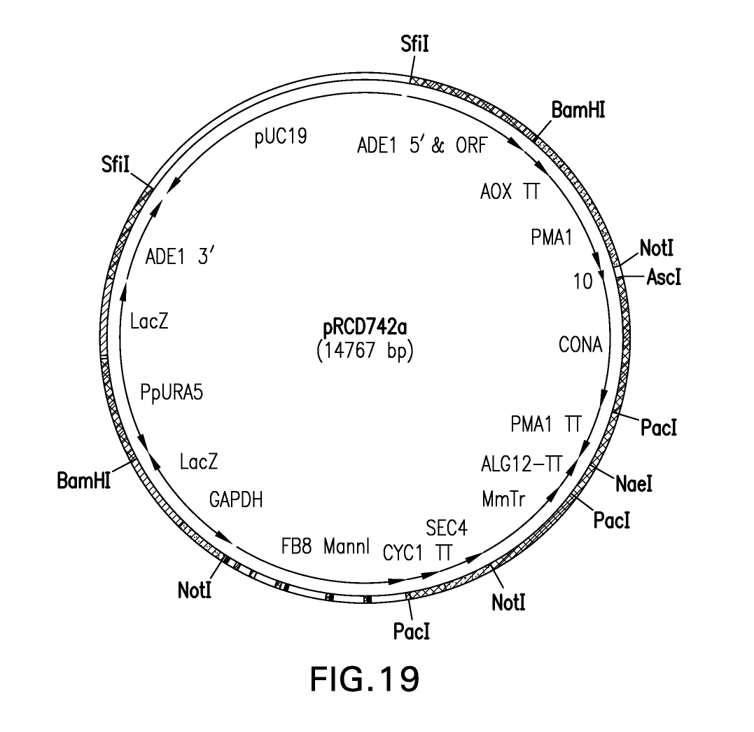

Strain YGLY16-3 was transformed with plasmid pRCD742a (See FIG. 19) to make strain RDP616-2. Plasmid pRCD742a (See FIG. 19) is a KINKO plasmid that integrates into the P. pastoris ADE1 gene without deleting the open reading frame encoding the Ade1p. The plasmid also contains the PpURA5 selectable marker and includes expression cassettes encoding the chimeric mouse alpha-1,2-mannosyltransferase (FB8 MannI), the chimeric human GlcNAc Transferase I (CONA10), and the full-length mouse Golgi UDP-GlcNAc transporter (MmSLC35A3). The plasmid is the same as plasmid pRCD742b except that the orientation of the expression cassette encoding the chimeric human GlcNAc Transferase I is in the opposite orientation. Transfection of plasmid pRCD742a into strain YGLY16-3 resulted in strain RDP616-2. This strain is capable of making glycoproteins that have GlcNAcMan₅GlcNAc₂ N-glycans.

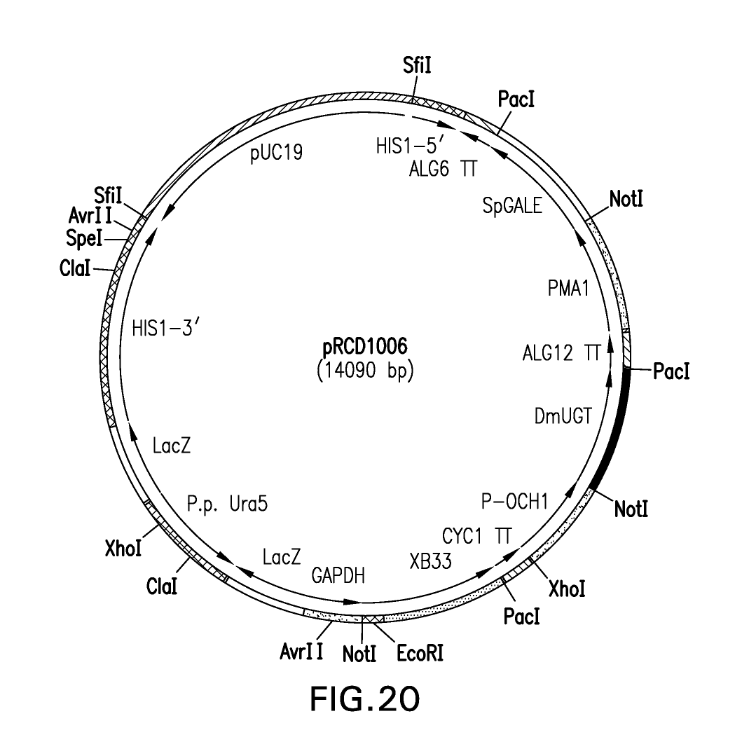

After counterselecting strain RDP616-2 to produce ura− strain RDP641-3, plasmid pRCD1006 was then transformed into the strain to make strain RDP666. Plasmid pRCD1006 (See FIG. 20) is a P. pastoris his1 knock-out plasmid that contains the PpURA5 gene as a selectable marker. The plasmid contains an expression cassette encoding a secretory pathway targeted fusion protein (XB33) comprising the first 58 amino acids of ScMnt1p (ScKre2p) (33) fused to the N-terminus of the human Galactosyl Transferase I catalytic domain (hGalTIβ43) under control of the PpGAPDH promoter; an expression cassette encoding the full length D. melanogaster Golgi UDP-galactose transporter (DmUGT) under control of the PpOCH1 promoter; and an expression cassette encoding the S. pombe UDP-galactose 4-epimerase (SpGALE) under control of the PpPMA1 promoter.

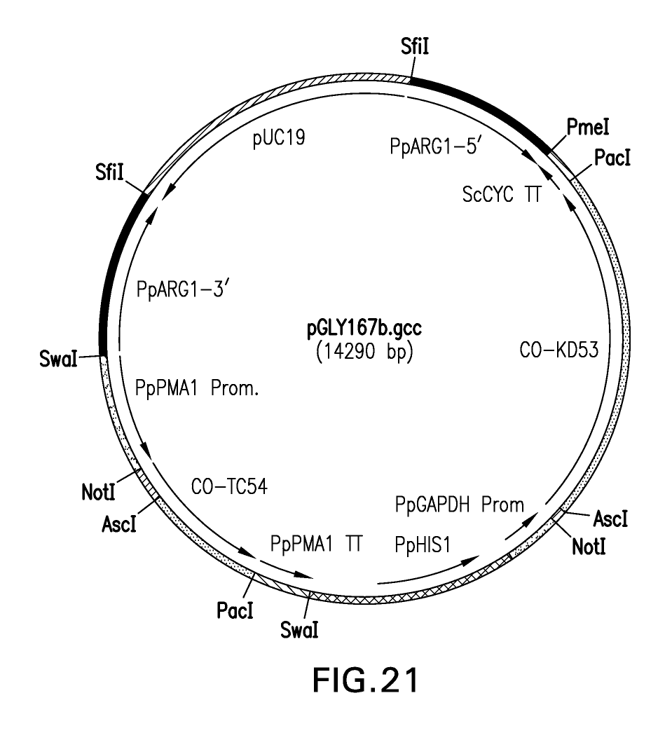

Strain RDP666 was transformed with plasmid pGLY167b to make strain RDP696-2. Plasmid pGLY167b (See FIG. 21) is a P. pastoris arg1 knock-out plasmid that contains the PpURA3 selectable marker. The plasmid contains an expression cassette encoding a secretory pathway targeted fusion protein (CO-KD53) comprising the first 36 amino acids of ScMnn2p (53) fused to N-terminus of the Drosophila melanogaster Mannosidase II catalytic domain (KD) under the control of PpGAPDH promoter and an expression cassette expressing a secretory pathway targeted fusion protein (CO-TC54) comprising the first 97 amino acids of ScMnn2p (54) fused to the N-terminus of the rat GlcNAc Transferase II catalytic domain (TC) under the control of the PpPMA1 promoter. Resulting strain RDP696-2 was subjected to chemostat selection (See Dykhuizen and Hartl, Microbiol. Revs. 47: 150-168 (1983) for a review of chemostat selection). Chemostat selection produced strain YGB02. Strain YGB02 can make glycoproteins that have N-glycans that have terminal galactose residues. In this strain, the mannosidase II catalytic domain (KD) and the GnT II (TC) were encoded by nucleic acid molecules that were codon-optimized for expression in Pichia pastoris (SEQ ID NO:64 and 59, respectively).

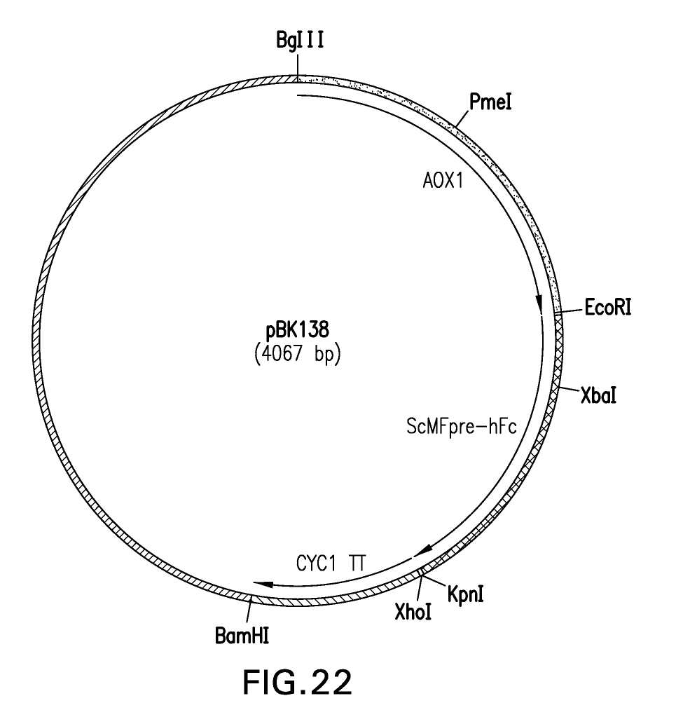

Strain YGB02 was transfected with plasmid pBK138 to produce strain PBP317-36. Plasmid pBK138 (See FIG. 22) is plasmid is a roll-in plasmid that integrates into the P. pastoris AOX1 promoter while duplicating the promoter. The plasmid contains an expression cassette encoding a fusion protein comprising the S. cerevisiae Alpha Mating Factor pre-signal sequence (SEQ ID NO:24) fused to the N-terminus of the human Fc antibody fragment (C-terminal 233-aa of a human IgG1 heavy chain; SEQ ID NO:66). The nucleic acid sequence encoding the S. cerevisiae Alpha Mating Factor pre-signal sequence is shown in SEQ ID NO:23 and the nucleic acid sequence encoding the C-terminal 233-aa of the human IgG1 Heavy chain is shown in SEQ ID NO:65).

### Example 5

Construction of strain YDX477 is shown in FIG. 11. The starting strain was YGLY16-3. Strain YGLY16-3 was transformed with plasmid pRCD742a (See FIG. 19) to make strain RDP616-2. Plasmid pRCD742a (See FIG. 19) is a KINKO plasmid that integrates into the P. pastoris ADE1 gene without deleting the open reading frame encoding the ade1p. The plasmid also contains the PpURA5 selectable marker and includes expression cassettes encoding the chimeric mouse alpha-1,2-mannosyltransferase (FB8 MannI), the chimeric human GlcNAc Transferase I (CONA10), and the full length mouse Golgi UDP-GlcNAc transporter (MmSLC35A3). The plasmid is the same as plasmid pRCD742b except that the orientation of the expression cassette encoding the chimeric human GlcNAc Transferase I is in the opposite orientation. Transfection of plasmid pRCD742a into strain YGLY16-3 resulted in strain RDP616-2. This strain is capable of making glycoproteins that have GlcNAcMan₅GlcNAc₂ N-glycans.

After counterselecting strain RDP616-2 to produce ura⁻ strain RDP641-4, plasmid pRCD1006 was then transformed into the strain to make strain RDP667-1. Plasmid pRCD1006 (See FIG. 20) is a P. pastoris his1 knock-out plasmid that contains the PpURA5 gene as a selectable marker. The plasmid contains an expression cassette encoding a secretory pathway targeted fusion protein (XB33) comprising the first 58 amino acids of ScMnt1p (ScKre2p) (33) fused to the N-terminus of the human Galactosyl Transferase I catalytic domain (hGalTIβ43) under control of the PpGAPDH promoter; an expression cassette encoding the full-length D. melanogaster Golgi UDP-galactose transporter (DmUGT) under control of the PpOCH1 promoter; and an expression cassette encoding the full-length S. pompe UDP-galactose 4-epimerase (SpGALE) under control of the PpPMA1 promoter.

Strain RDP667-1 was transformed with plasmid pGLY167b to make strain RDP697-1. Plasmid pGLY167b (See FIG. 21) is a P. pastoris arg1 knock-out plasmid that contains the PpURA3 selectable marker. The plasmid contains an expression cassette encoding a secretory pathway targeted fusion protein (CO-KD53) comprising the first 36 amino acids of ScMnn2p (53) fused to N-terminus of the Drosophila melanogaster Mannosidase II catalytic domain (KD) under the control of PpGAPDH promoter and an expression cassette expressing a secretory pathway targeted fusion protein (CO-TC54) comprising the first 97 amino acids of ScMnn2p (54) fused to the N-terminus of the rat GlcNAc Transferase 11 catalytic domain under the control of the PpPMA1 promoter. The nucleic acid molecules encoding the mannosidase H and GnT II catalytic domains were codon-optimized for expression in Pichia pastoris (SEQ ID NO:64 and 59, respectively). This strain can make glycoproteins that have N-glycans that have terminal galactose residues.

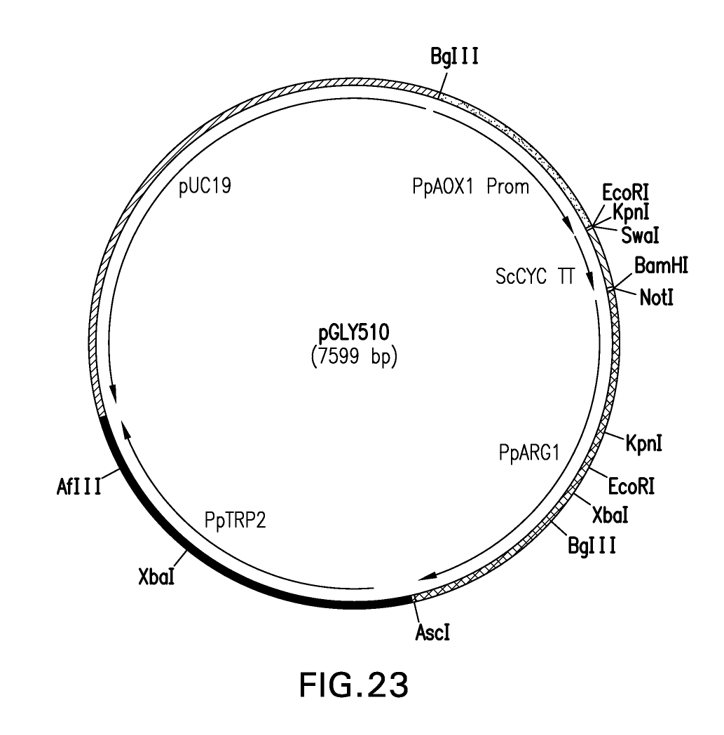

Strain RDP697-1 was transformed with plasmid pGLY510 to make strain YDX414. Plasmid pGLY510 (See FIG. 23) is a roll-in plasmid that integrates into the P. pastoris TRP2 locus while duplicating the gene and contains an AOX1 promoter-ScCYC1 terminator expression cassette as well as the PpARG1 selectable marker.

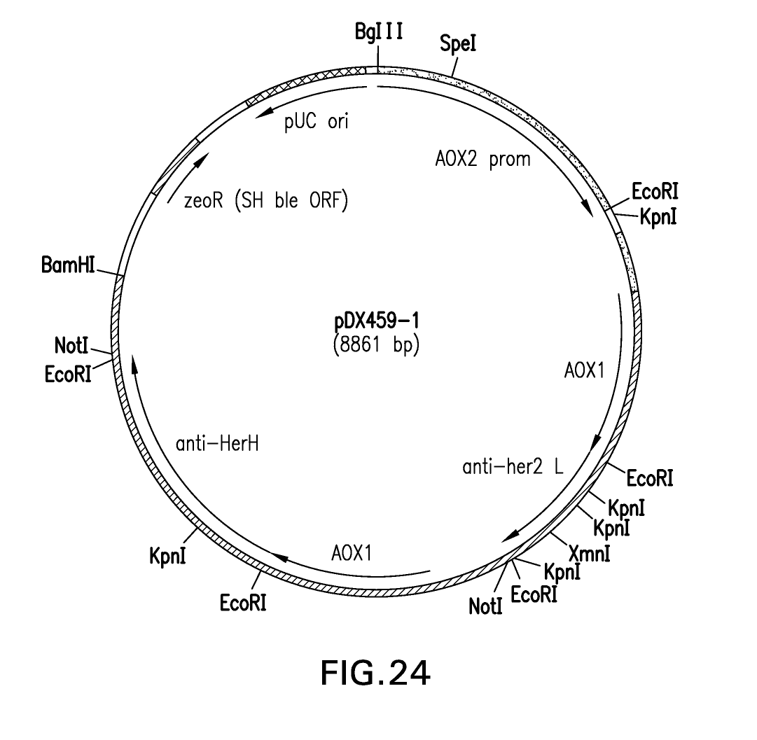

Strain YDX414 was transformed with plasmid pDX459-1 (mAb-A) to make strain YDX458. Plasmid pDX459-1 (See FIG. 24) is a roll-in plasmid that targets and integrates into the P. pastoris AOX2 promoter and contains the ZeoR while duplicating the promoter. The plasmid contains separate expression cassettes encoding an anti-HER2 antibody heavy chain and an anti-HER2 antibody light chain (SEQ ID NOs:68 and 70, respectively), each fused at the N-terminus to the Aspergillus niger alpha-amylase signal sequence (SEQ ID NO:26) and controlled by the P. pastoris AOX1 promoter. The nucleic acid sequences encoding the heavy and light chains are shown in SEQ ID NOs:67 and 69, respectively, and the nucleic acid sequence encoding the Aspergillus niger alpha-amylase signal sequence is shown in SEQ ID NO:25.

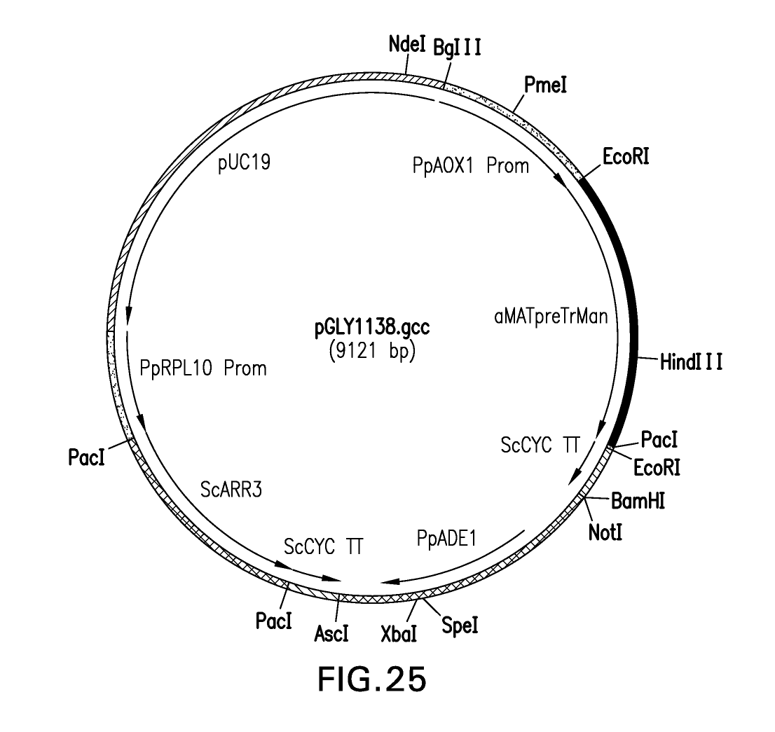

Strain YDX458 was transformed with plasmid pGLY1138 to make strain YDX477. Plasmid pGLY1138 (See FIG. 25) is a roll-in plasmid that integrates into the P. pastoris ADE1 locus while duplicating the gene. The plasmid contains a ScARR3 selectable marker gene cassette. The ARR3 gene from S. cerevisiae confers arsenite resistance to cells that are grown in the presence of arsenite (Bobrowicz et al., Yeast, 13:819-828 (1997); Wysocki et al., J. Biol. Chem. 272:30061-30066 (1997)). The plasmid contains an expression cassette encoding a secreted fusion protein comprising the S. cerevisiae alpha factor pre signal sequence (SEQ ID NO:24) fused to the N-terminus of the Trichoderma reesei (MNS1) catalytic domain (SEQ ID NO:56 encoded by the nucleotide sequence in SEQ ID NO:55) under the control of the PpAOX1 promoter. The fusion protein is secreted into the culture medium.

Table of Sequences

| SEQ | | |
|---|---|---|
| ID | | |
| NO: | Description | Sequence |
| 1 | PCR | GCCGCGACCTGAGCC GCCTGCCCCAAC |
| | primer | |
| | RCD192 | |
| 2 | PCR | CTAGCTCGGTGTCCCGATGTCCACTGT |
| | primer | |
| | RCD186 | |
| 3 | PCR | CTTAGGCGCGCCGGCCGCGACCTGAGCCGCCTGCCC |
| | primer | |
| | RCD198 | |
| 4 | PCR | GGGGCATATCTGCCGCCCATC |
| | primer | |
| | RCD201 | |
| 5 | PCR | GATGGGCGGCAGATATGCCCC |
| | primer | |
| | RCD200 | |
| 6 | PCR | CTTCTTAATTAACTAGCTCGGTGTCCCGATGTCCAC |
| | primer | |
| | RCD199 | |
| 7 | PCR | GGCTCGAGCGGCCGCCACCATGAATAGCATACACATGAACGCCAATA |
| | primer | CG |
| | DmUGT- | |
| | 5′ | |
| 8 | PCR | CCCTCGAGTTAATTAACTAGACGCGCGGCAGCAGCTTCTCCTCATCG |
| | primer | |
| | DmUGT- | |
| | 3′ | |
| 9 | PCR | ATGACTGGTGTTCATGAAGGG |
| | primer | |
| | GALE2-L | |
| 10 | PCR | TTACTTATATGTCTTGGTATG |
| | primer | |
| | GALE2-R | |
| 11 | PCR | GCGGCCGCATGACTGGTGTTCATGAAGGGACTGTGTTGGTTACTGGC |
| | primer | GGCGCTGGTTATATA GGTTCTCATACGTGCGTTGTTTTGTTAGAAAA |
| | GD1 | |
| 12 | PCR | TTAATTAATTACTTATAT GTCTTGGTATG |
| | primer | |
| | GD2 | |
| 13 | PCR | TTAGCGGCCGCAGGAATGACTAAATCTCATTCA |
| | primer | |
| | PB158 | |
| 14 | PCR | AACTTAATTAAGCTTATAATTCATATAGACAGC |
| | primer | |
| | PB159 | |
| 15 | PCR | TTAGCGGCCGC |
| | primer | |
| | PB156 | |
| 16 | PCR | AACTTAATTAA |
| | primer | |
| | PB157 | |
| 17 | PCR | TTAGCGGCCGCAGGAATGACTGCTGAAGAATT |
| | primer | |
| | PB160 | |
| 18 | PCR | AACTTAATTAAGCTTACAGTCTTTGTAGATAATC |
| | primer | |
| | PB161 | |
| 19 | DNA | ATGCTGCTTACCAAAAGGTTTTCAAAGCTGTTCAAGCTGACGTTCATA |
| | encodes | GTTTTGATATTGTGCGGGCTGTTCGTCATTACAAACAAATACATGGAT |
| | Mnn2 | GAGAACACGTCG |
| | leader (53) | |
| 20 | Mnn2 | MLLTKRFSKLFKLTFIVLILCGLFVITNKYMDENTS |
| | leader (53) | |
| 21 | DNA | ATGCTGCTTACCAAAAGGTTTTCAAAGCTGTTCAAGCTGACGTTCATA |
| | encodes | GTTTTGATATTGTGCGGGCTGTTCGTCATTACAAACAAATACATGGAT |
| | Mnn2 | GAGAACACGTCGGTCAAGGAGTACAAGGAGTACTTAGACAGATATG |
| | leader (54) | TCCAGAGTTACTCCAATAAGTATTCATCTTCCTCAGACGCCGCCAGCG |
| | The last 9 | CTGACGATTCAACCCCATTGAGGGACAATGATGAGGCAGGCAATGA |
| | nucleotides | AAAGTTGAAAAGCTTCTACAACAACGTTTTCAACTTTCTAATGGTTGA |
| | are the | TTCGCCCGGGCGCGCC |
| | linker | |
| | containing | |
| | the AscI | |
| | restriction | |
| | site) | |
| 22 | Mnn2 | MLLTKRFSKLFKLTFIVLILCGLFVITNKYMDENTSVKEYKEYLDRYVQS |
| | leader (54) | YSNKYSSSSD |
| | | AASADDSTPLRDNDEAGNEKLKSFYNNVFNFLMVDSPGRA |
| 23 | DNA | ATG AGA TTC CCA TCC ATC TTC ACT GCT GTT TTG TTC GCT GCT |
| | encodes S. | TCT TCT GCT TTG GCT |
| | cerevisiae | |
| | Mating | |
| | Factor pre | |
| | signal | |
| | sequence | |
| 24 | S. | MRFPSIFTAVLFAASSALA |
| | cerevisiae | |
| | Mating | |
| | Factor pre | |
| | signal | |
| | sequence | |
| 25 | DNA | ATGGTTGCTT GGTGGTCCTT GTTCTTGTAC GGATTGCAAG |
| | encodes | TTGCTGCTCC AGCTTTGGCT |
| | alpha | |
| | amylase | |
| | signal | |
| | sequence | |
| | (from | |
| | Aspergillus | |
| | niger α- | |
| | amylase) | |
| | (DNA) | |
| 26 | Alpha | MVAWWSLFLY GLQVAAPALA |
| | amylase | |
| | signal | |
| | sequence | |
| | (from | |
| | Aspergillus | |
| | niger α- | |
| | amylase) | |
| 27 | DNA | ATGCCCAGAAAAATATTTAACTACTTCATTTTGACTGTATTCATGGCA |
| | encodes Pp | ATTCTTGCTATTGTTTTACAATGGTCTATAGAGAATGGACATGGGCGC |
| | SEC 12 | GCC |
| | (10) | |
| | The last 9 | |
| | nucleotides | |
| | are the | |
| | linker | |
| | containing | |
| | the AscI | |
| | restriction | |
| | site used | |
| | for fusion | |
| | to proteins | |
| | of interest. | |
| 28 | Pp SEC12 | MPRKIFNYFILTVFMAILAIVLQWSIENGHGRA |
| | (10) | |
| 29 | DNA | ATGGCCCTCTTTCTCAGTAAGAGACTGTTGAGATTTACCGTCATTGCA |
| | encodes | GGTGCGGTTATTGTTCTCCTCCTAACATTGAATTCCAACAGTAGAACT |
| | ScMnt1 | CAGCAATATATTCCGAGTTCCATCTCCGCTGCATTTGATTTTACCTCA |
| | (Kre2) (33) | GGATCTATATCCCCTGAACAACAAGTCATCGGGCGCGCC |
| 30 | ScMnt1 | MALFLSKRLLRFTVIAGAVIVLLLTLNSNSRTQQYIPSSISAAFDFTSGSISP |
| | (Kre2) (33) | EQQVIGRA |
| 31 | DNA | ATGAACACTATCCACATAATAAAATTACCGCTTAACTACGCCAACTA |
| | encodes | CACCTCAATGAAACAAAAAATCTCTAAATTTTTCACCAACTTCATCCT |
| | ScSEC12 | TATTGTGCTGCTTTCTTACATTTTACAGTTCTCCTATAAGCACAATTTG |
| | (8) | CATTCCATGCTTTTCAATTACGCGAAGGACAATTTTCTAACGAAAAG |
| | The last 9 | AGACACCATCTCTTCGCCCTACGTAGTTGATGAAGACTTACATCAAA |
| | nucleotides | CAACTTTGTTTGGCAACCACGGTACAAAAACATCTGTACCTAGCGTA |
| | are the | GATTCCATAAAAGTGCATGGCGTGGGGCGCGCC |
| | linker | |
| | containing | |
| | the AscI | |
| | restriction | |
| | site used | |
| | for fusion | |
| | to proteins | |
| | of interest | |
| 32 | ScSEC12 | MNTIHIIKLPLNYANYTSMKQKISKFFTNFILIVLLSYILQFSYKHNLHSML |
| | (8) | FNYAKDNFLTKRDTISSPYVVDEDLHQTTLFGNHGTKTSVPSVDSIKVHG |
| | | VGRA |
| 33 | DNA | ATGTCTGCCAACCTAAAATATCTTTCCTTGGGAATTTTGGTGTTTCAG |
| | encodes | ACTACCAGTCTGGTTCTAACGATGCGGTATTCTAGGACTTTAAAAGA |
| | MmSLC35 | GGAGGGGCCTCGTTATCTGTCTTCTACAGCAGTGGTTGTGGCTGAATT |
| | A3 UDP- | TTTGAAGATAATGGCCTGCATCTTTTTAGTCTACAAAGACAGTAAGT |
| | GlcNAc | GTAGTGTGAGAGCACTGAATAGAGTACTGCATGATGAAATTCTTAAT |
| | transporter | AAGCCCATGGAAACCCTGAAGCTCGCTATCCCGTCAGGGATATATAC |
| | | TCTTCAGAACAACTTACTCTATGTGGCACTGTCAAACCTAGATGCAG |
| | | CCACTTACCAGGTTACATATCAGTTGAAAATACTTACAACAGCATTA |
| | | TTTTCTGTGTCTATGCTTGGTAAAAAATTAGGTGTGTACCAGTGGCTC |
| | | TCCCTAGTAATTCTGATGGCAGGAGTTGCTTTTGTACAGTGGCCTTCA |
| | | GATTCTCAAGAGCTGAACTCTAAGGACCTTTCAACAGGCTCACAGTT |
| | | TGTAGGCCTCATGGCAGTTCTCACAGCCTGTTTTTCAAGTGGCTTTGC |
| | | TGGAGTTTATTTTGAGAAAATCTTAAAAGAAACAAAACAGTCAGTAT |
| | | GGATAAGGAACATTCAACTTGGTTTCTTTGGAAGTATATTTGGATTAA |
| | | TGGGTGTATACGTTTATGATGGAGAATTGGTCTCAAAGAATGGATTTT |
| | | TTCAGGGATATAATCAACTGACGTGGATAGTTGTTGCTCTGCAGGCA |
| | | CTTGGAGGCCTTGTAATAGCTGCTGTCATCAAATATGCAGATAACAT |
| | | TTTAAAAGGATTTGCGACCTCCTTATCCATAATATTGTCAACAATAAT |
| | | ATCTTATTTTTGGTTGCAAGATTTTGTGCCAACCAGTGTCTTTTTCCTT |
| | | GGAGCCATCCTTGTAATAGCAGCTACTTTCTTGTATGGTTACGATCCC |
| | | AAACCTGCAGGAAATCCCACTAAAGCATAG |
| 34 | MmSLC35 | MSANLKYLSLGILVFQTTSLVLTMRYSRTLKEEGPRYLSSTAVVVAEFLK |
| | A3 UDP- | IMACIFLVYKDSKCSVRALNRVLHDEILNKPMETLKLAIPSGIYTLQNNLL |
| | GlcNAc | YVALSNLDAATYQVTYQLKILTTALFSVSMLGKKLGVYQWLSLVILMA |
| | transporter | GVAFVQWPSDSQELNSKDLSTGSQFVGLMAVLTACFSSGFAGVYFEKIL |
| | | KETKQSVWIRNIQLGFFGSIFGLMGVYVYDGELVSKNGFFQGYNQLTWI |
| | | VVALQALGGLVIAAVIKYADNILKGFATSLSIILSTIISYFWLQDFVPTSVF |
| | | FLGAILVIAATFLYGYDPKPAGNPTKA |
| 35 | DNA | ATGACTGGTGTTCATGAAGGGACTGTGTTGGTTACTGGCGGCGCTGG |
| | encodes | TTATATAGGTTCTCATACGTGCGTTGTTTTGTTAGAAAAAGGATATGA |
| | SpGALE | TGTTGTAATTGTCGATAATTTATGCAATTCTCGCGTTGAAGCCGTGCA |
| | | CCGCATTGAAAAACTCACTGGGAAAAAAGTCATATTCCACCAGGTGG |
| | | ATTTGCTTGATGAGCCAGCTTTGGACAAGGTCTTCGCAAATCAAAAC |
| | | ATATCTGCTGTCATTCATTTTGCTGGTCTCAAAGCAGTTGGTGAATCT |
| | | GTACAGGTTCCTTTGAGTTATTACAAAAATAACATTTCCGGTACCATT |
| | | AATTTAATAGAGTGCATGAAGAAGTATAATGTACGTGACTTCGTCTTT |
| | | TCTTCATCTGCTACCGTGTATGGCGATCCTACTAGACCTGGTGGTACC |
| | | ATTCCTATTCCAGAGTCATGCCCTCGTGAAGGTACAAGCCCATATGG |
| | | TCGCACAAAGCTTTTCATTGAAAATATCATTGAGGATGAGACCAAGG |
| | | TGAACAAATCGCTTAATGCAGCTTTATTACGCTATTTTAATCCCGGAG |
| | | GTGCTCATCCCTCTGGTGAACTCGGTGAAGATCCTCTTGGCATCCCTA |
| | | ATAACTTGCTTCCTTATATCGCGCAAGTTGCTGTAGGAAGATTGGATC |
| | | ATTTGAATGTATTTGGCGACGATTATCCCACATCTGACGGTACTCCAA |
| | | TTCGTGACTACATTCACGTATGCGATTTGGCAGAGGCTCATGTTGCTG |
| | | CTCTCGATTACCTGCGCCAACATTTTGTTAGTTGCCGCCCTTGGAATT |
| | | TGGGATCAGGAACTGGTAGTACTGTTTTTCAGGTGCTCAATGCGTTTT |
| | | CGAAAGCTGTTGGAAGAGATCTTCCTTATAAGGTCACCCCTAGAAGA |
| | | GCAGGGGACGTTGTTAACCTAACCGCCAACCCCACTCGCGCTAACGA |
| | | GGAGTTAAAATGGAAAACCAGTCGTAGCATTTATGAAATTTGCGTTG |
| | | ACACTTGGAGATGGCAACAGAAGTATCCCTATGGCTTTGACCTGACC |
| | | CATACCAAGACATATAAGTAA |
| 36 | SpGALE | MTGVHEGTVLVTGGAGYIGSHTCVVLLEKGYDVVIVDNLCNSRVEAVH |
| | | RIEKLTGKKVIFHQVDLLDEPALDKVFANQNISAVIHFAGLIKAVGESVQV |
| | | PLSYYKNNISGTINLIECMKKYNVRDFVFSSSATVYGDPTRPGGTIPIPESC |
| | | PREGTSPYGRTKLFIENIIEDETKVNKSLNAALLRYFNPGGAHPSGELGED |
| | | PLGIPNNLLPYIAQVAVGRLDHLNVFGDDYPTSDGTPIRDYIHVCDLAEA |
| | | HVAALDYLRQHFVSCRPWNLGSGTGSTVFQVLNAFSKAVGRDLPYKVT |
| | | PRRAGDVVNLTANPTRANEELKWKTSRSIYEICVDTWRWQQKYPYGFD |
| | | LTHTKTYK |
| 37 | DNA | ATGAATAGCATACACATGAACGCCAATACGCTGAAGTACATCAGCCT |
| | encodes | GCTGACGCTGACCCTGCAGAATGCCATCCTGGGCCTCAGCATGCGCT |
| | DmUGT | ACGCCCGCACCCGGCCAGGCGACATCTTCCTCAGCTCCACGGCCGTA |
| | | CTCATGGCAGAGTTCGCCAAACTGATCACGTGCCTGTTCCTGGTCTTC |
| | | AACGAGGAGGGCAAGGATGCCCAGAAGTTTGTACGCTCGCTGCACA |
| | | AGACCATCATTGCGAATCCCATGGACACGCTGAAGGTGTGCGTCCCC |
| | | TCGCTGGTCTATATCGTTCAAAACAATCTGCTGTACGTCTCTGCCTCC |
| | | CATTTGGATGCGGCCACCTACCAGGTGACGTACCAGCTGAAGATTCT |
| | | CACCACGGCCATGTTCGCGGTTGTCATTCTGCGCCGCAAGCTGCTGA |
| | | ACACGCAGTGGGGTGCGCTGCTGCTCCTGGTGATGGGCATCGTCCTG |
| | | GTGCAGTTGGCCCAAACGGAGGGTCCGACGAGTGGCTCAGCCGGTG |
| | | GTGCCGCAGCTGCAGCCACGGCCGCCTCCTCTGGCGGTGCTCCCGAG |
| | | CAGAACAGGATGCTCGGACTGTGGGCCGCACTGGGCGCCTGCTTCCT |
| | | CTCCGGATTCGCGGGCATCTACTTTGAGAAGATCCTCAAGGGTGCCG |
| | | AGATCTCCGTGTGGATGCGGAATGTGCAGTTGAGTCTGCTCAGCATT |
| | | CCCTTCGGCCTGCTCACCTGTTTCGTTAACGACGGCAGTAGGATCTTC |
| | | GACCAGGGATTCTTCAAGGGCTACGATCTGTTTGTCTGGTACCTGGTC |
| | | CTGCTGCAGGCCGGCGGTGGATTGATCGTTGCCGTGGTGGTCAAGTA |
| | | CGCGGATAACATTCTCAAGGGCTTCGCCACCTCGCTGGCCATCATCA |
| | | TCTCGTGCGTGGCCTCCATATACATCTTCGACTTCAATCTCACGCTGC |
| | | AGTTCAGCTTCGGAGCTGGCCTGGTCATCGCCTCCATATTTCTCTACG |
| | | GCTACGATCCGGCCAGGTCGGCGCCGAAGCCAACTATGCATGGTCCT |
| | | GGCGGCGATGAGGAGAAGCTGCTGCCGCGCGTCTAG |
| 38 | DmUGT | MNSIHMNANTLKYISLLTLTLQNAILGLSMRYARTRPGDIFISSTAVLMA |
| | | EFAKLITCLELVFNEEGKDAQKEVRSLHKTIIANPMDTLKVCVPSLVYIVQ |
| | | NNLLYVSASHLDAATYQVTYQLKILTTAMFAVVILRRKLLNTQWGALLL |
| | | LVMGIVLVQLAQTEGPTSGSAGGAAAAATAASSGGAPEQNRMLGLWA |
| | | ALGACFLSGFAGIYFEKILKGAEISVWMRNVQLSLLSIPFGLLTCFVNDGS |
| | | RIFDQGFFKGYDLFVWYLVLLQAGGGLIVAVVVKYADNILKGFATSLAIII |
| | | SCVASIYIFDFNLTLQFSFGAGLVIASIFLYGYDPARSAPKPTMHGPGGDE |
| | | EKLLPRV |
| 39 | DNA | ATGACTAAATCTCATTCAGAAGAAGTGATTGTACCTGAGTTCAATTCT |
| | encodes | AGCGCAAAGGAATTACCAAGACCATTGGCCGAAAAGTGCCCGAGCA |
| | ScGAL1 | TAATTAAGAAATTTATAAGCGCTTATGATGCTAAACCGGATTTTGT |
| | | TGCTAGATCGCCTGGTAGAGTCAATCTAATTGGTGAACATATTGATT |
| | | ATTGTGACTTCTCGGTTTTACCTTTAGCTATTGATTTTGATATGCTTTG |
| | | CGCCGTCAAAGTTTTGAACGAGAAAAATCCATCCATTACCTTAA |
| | | TAAATGCTGATCCCAAATTTGCTCAAAGGAAGTTCGATTTGCCGTTG |
| | | GACGGTTCTTATGTCACAATTGATCCTTCTGTGTCGGACTGGTCTAAT |
| | | TACTTTAAATGTGGTCTCCATGTTGCTCACTCTTTTCTAAAGAAA |
| | | CTTGCACCGGAAAGGTTTGCCAGTGCTCCTCTGGCCGGGCTGCAAGT |
| | | CTTCTGTGAGGGTGATGTACCAACTGGCAGTGGATTGTCTTCTTCGGC |
| | | CGCATTCATTTGTGCCGTTGCTTTAGCTGTTGTTAAAGCGAATAT |
| | | GGGCCCTGGTTATCATATGTCCAAGCAAAATTTAATGCGTATTACGG |
| | | TCGTTGCAGAACATTATGTTGGTGTTAACAATGGCGGTATGGATCAG |
| | | GCTGCCTCTGTTTGCGGTGAGGAAGATCATGCTCTATACGTTGAGTTC |
| | | AAACCGCAGTTGAAGGCTACTCCGTTTAAATTTCCGCAATTAAAAAA |
| | | CCATGAAATTAGCTTTGTTATTGCGAACACCCTTGTTGTATCTAACAA |
| | | GTTTGAAACCGCCCCAACCAACTATAATTTAAGAGTGGTAGAAGTCA |
| | | CTACAGCTGCAAATGTTTTAGCTGCCACGTACGGTGTTGTTTTACTTT |
| | | CTGGAAAAGAAGGATCGAGCACGAATAAAGGTAATCTAAGAGATTT |
| | | CATGAACGTTTATTATGCCAGATATCACAACATTTCCACACCCTGGA |
| | | ACGGCGATATTGAATCCGGCATCGAACGGTTAACAAAGATGCTAGTA |
| | | CTAGTTGAAGAGTCTCTCGCCAATAAGAAACAGGGCTTTAGTGTTGA |
| | | CGATGTCGCACAATCCTTGAATTGTTCTCGCGAAGAATTCACAAGAG |
| | | ACTACTTAACAACATCTCCAGTGAGATTTCAAGTCTTAAAGCTATATC |
| | | AGAGGGCTAAGCATGTGTATTCTGAATCTTTAAGAGTCTTGAAGGCT |
| | | GTGAAATTAATGACTACAGCGAGCTTTACTGCCGACGAAGACTTTTT |
| | | CAAGCAATTTGGTGCCTTGATGAACGAGTCTCAAGCTTCTTGCGATA |
| | | AACTTTACGAATGTTCTTGTCCAGAGATTGACAAAATTTGTTCCATTG |
| | | CTTTGTCAAATGGATCATATGGTTCCCGTTTGACCGGAGCTGGCTGGG |
| | | GTGGTTGTACTGTTCACTTGGTTCCAGGGGGCCCAAATGGCAACATA |
| | | GAAAAGGTAAAAGAAGCCCTTGCCAATGAGTTCTACAAGGTCAAGT |
| | | ACCCTAAGATCACTGATGCTGAGCTAGAAAATGCTATCATCGTCTCT |
| | | AAACCAGCATTGGGCAGCTGTCTATATGAATTATAA |
| 40 | ScGAL1 | MTKSHSEEVIVPEFNSSAKELPRPLAEKCPSIIKKFISAYDAKPDFVARSPG |
| | | RVNLIGEHIDYCDFSVLPLAIDFDMLCAVKVLNEKNPSITLINADPKFAQR |
| | | KFDLPLDGSYVTIDPSVSDWSNYFKCGLHVAHSFLKKLAPERFASAPLAG |
| | | LQVFCEGDVPTGSGLSSSAAFICAVALAVVKANMGPGYHMSKQNLMRI |
| | | TVVAEHYVGVNNGGMDQAASVCGEEDHALYVEFKPQLKATPFKFPQL |
| | | KNHEISFVIANTLVVSNKFETAPTNYNLRVVEVTTAANVLAATYGVVLL |
| | | SGKEGSSTNKGNLRDFMNVYYARYHNISTPWNGDIESGIERLTKMLVLV |
| | | EESLANKKQGFSVDDVAQSLNCSREEFTRDYLTTSPVRFQVLKLYQRAK |
| | | HVYSESLRVLKAVKLMTTASFTADEDFFKQFGALMNESQASCDKLYECS |
| | | CPEIDKICSIALSNGSYGSRLTGAGWGGCTVHLVPGGPNGNIEKVK |
| | | EALANEFYKVKYPKITDAELENAIIVSKPALGSCLYEL |
| 41 | DNA | ATGACTGCTGAAGAATTTGATTTTTCTAGCCATTCCCATAGACGTTAC |
| | encodes | AATCCACTAACCGATTCATGGATCTTAGTTTCTCCACACAGAGCTAA |
| | ScGAL7 | AAGACCTTGGTTAGGTCAACAGGAGGCTGCTTACAAGCCCACAGCTC |
| | | CTTTGTATGATCCAAAATGCTATCTATGTCCTGGTAACAAAAGAGCT |
| | | ACTGGTAACCTAAACCCAAGATATGAATCAACGTATATTTTCCCCAA |
| | | TGATTATGCTGCCGTTAGCGATCAACCTATTTTACCACAGAATGATTC |
| | | CAATGAGGATAATCTTAAAAATAGGCTGCTTAAAGTGCAATCTGTGA |
| | | GAGGCAATTGTTTCGTCATATGTTTTAGCCCCAATCATAATCTAACCA |
| | | TTCCACAAATGAAACAATCAGATCTGGTTCATATTGTTAATTCTTGGC |
| | | AAGCATTGACTGACGATCTCTCCAGAGAAGCAAGAGAAAATCATAA |
| | | GCCTTTCAAATATGTCCAAATATTTGAAAACAAAGGTACAGCCATGG |
| | | GTTGTTCCAACTTACATCCACATGGCCAAGCTTGGTGCTTAGAATCCA |
| | | TCCCTAGTGAAGTTTCGCAAGAATTGAAATCTTTTGATAAATATAAA |
| | | CGTGAACACAATACTGATTTGTTTGCCGATTACGTCAAATTAGAATC |
| | | AAGAGAGAAGTCAAGAGTCGTAGTGGAGAATGAATCCTTTATTGTTG |
| | | TTGTTCCATACTGGGCCATCTGGCCATTTGAGACCTTGGTCATTTCAA |
| | | AGAAGAAGCTTGCCTCAATTAGCCAATTTAACCAAATGGCGAAGGAG |
| | | GACCTCGCCTCGATTTTAAAGCAACTAACTATTAAGTATGATAATTTA |
| | | TTTGAAACGAGTTTCCCATACTCAATGGGTATCCATCAGGCTCCTTTG |
| | | AATGCGACTGGTGATGAATTGAGTAATAGTTGGTTTCACATGCATTTC |
| | | TACCCACCTTTACTGAGATCAGCTACTGTTCGGAAATTCTTGGTTGGT |
| | | TTTGAATTGTTAGGTGAGCCTCAAAGAGATTTAATTTCGGAACAAGC |
| | | TGCTGAAAAACTAAGAAATTTAGATGGTCAGATTCATTATCTACAAA |
| | | GACTATAA |
| 42 | ScGAL7 | MTAEEFDFSSHSHRRYNPLTDSWILVSPHRAKRPWLGQQEAAYKPTAPL |
| | | YDPKCYLCPGNKRATGNLNPRYESTYIFPNDYAAVSDQPILPQNDSNED |
| | | NLKNRLLKVQSVRGNCEVICFSPNHNLTIPQMKQSDLVHIVNSWQALTD |
| | | DLSREARENHKPFKYVQIFENKGTAMGCSNLHPHGQAWCLESIPSEVSQ |
| | | ELKSFDKYKREHNTDLFADYVKLESREKSRVVVENESFIVVVPYWAIWP |
| | | FETLVISKKKLASISQFNQMAKEDLASILKQLTTKYDNLFETSFPYSMGIH |
| | | QAPLNATGDELSNSWFHMHEYPPLLRSATVRKFLVGFELLGEPQRDLISE |
| | | QAAEKLRNLDGQIHYLQRL |
| 43 | DNA | ATGGCAGTTGAGGAGAACAATGTGCCTGTTGTTTCACAGCAACCCCA |
| | encodes | AGCTGGTGAAGACGTGATCTCTTCACTCAGTAAAGATTCCCATTTAA |
| | ScGal | GCGCACAATCTCAAAAGTATTCCAATGATGAATTGAAAGCCGGTGA |
| | permease | GTCAGGGCCTGAAGGCTCCCAAAGTGTTCCTATAGAGATACCCAAGA |
| | | AGCCCATGTCTGAATATGTTACCGTTTCCTTGCTTTGTTTGTGTGTTGC |
| | | CTTCGGCGGCTTCATGTTTGGCTGGGATACCAGTACTATTTCTGGGTT |
| | | TGTTGTCCAAACAGACTTTTTGAGAAGGTTTGGTATGAAACATAAGG |
| | | ATGGTACCCACTATTTGTCAAACGTCAGAACAGGTTTAATCGTCGCC |
| | | ATTTTCAATATTGGCTGTGCCTTTGGTGGTATTATACTTTCCAAAGGT |
| | | GGAGATATGTATGGCCGTAAAAAGGGTCTTTCGATTGTCGTCTCGGT |
| | | TTATATAGTTGGTATTATCATTCAAATTGCCTCTATCAACAAGTGGTA |
| | | CCAATATTTCATTGGTAGAATCATATCTGGTTTGGGTGTCGGCGGCAT |
| | | CGCTGTCTTATGTCCTATGTTGATCTCTGAAATTGCTCCAAAGCACTT |
| | | GAGAGGCACACTAGTTTCTTGTTATCAGCTGATGATTACTGCAGGTAT |
| | | CTTTTTGGGCTACTGTACTAATTACGGTACAAAGAGCTATTCGAACTC |
| | | AGTTCAATGGAGAGTTCCATTAGGGCTATGTTTCGCTTGGTCATTATT |
| | | TATGATTGGCGCTTTGACGTTAGTTCCTGAATCCCCACGTTATTTATG |
| | | TGAGGTGAATAAGGTAGAAGACGCCAAGCGTTCCATTGCTAAGTCTA |
| | | ACAAGGTGTCACCAGAGGATCCTGCCGTCCAGGCCGAGTTAGATCTG |
| | | ATCATGGCCGGTATAGAAGCTGAAAAACTGGCTGGCAATGCGTCCTG |
| | | GGGGGAATTATTTTCCACCAAGACCAAAGTATTTCAACGTTTGTTGAT |
| | | GGGTGTGTTTGTTCAAATGTTCCAACAATTAACCGGTAACAATTATTT |
| | | TTTCTACTACGGTACCGTTATTTTCAAGTCAGTTGGCCTGGATGATTC |
| | | CTTTGAAACATCCATTGTCATTGGTGTAGTCAACTTTGCCTCCACTTT |
| | | CTTTAGTTTGTGGACTGTCGAAAACTTGGGGCGTCGTAAATGTTTACT |
| | | TTTGGGCGCTGCCACTATGATGGCTTGTATGGTCATCTACGCCTCTGT |
| | | TGGTGTTACTAGATTATATCCTCACGGTAAAAGCCAGCCATCTTCTAA |
| | | AGGTGCCGGTAACTGTATGATTGTCTTTACCTGTTTTTATATTTTCTGT |
| | | TATGCCACAACCTGGGCGCCAGTTGCCTGGGTCATCACAGCAGAATC |
| | | ATTCCCACTGAGAGTCAAGTCGAAATGTATGGCGTTGGCCTCTGCTTC |
| | | CAATTGGGTATGGGGGTTCTTGATTGCATTTTTCACCCCATTCATCAC |
| | | ATCTGCCATTAACTTCTACTACGGTTATGTCTTCATGGGCTGTTTGGT |
| | | TGCCATGTTTTTTTATGTCTTTTTCTTTGTTCCAGAAACTAAAGGCCTA |
| | | TCGTTAGAAGAAATTCAAGAATTATGGGAAGAAGGTGTTTTACCTTG |
| | | GAAATCTGAAGGCTGGATTCCTTCATCCAGAAGAGGTAATAATTACG |
| | | ATTTAGAGGATTTACAACATGACGACAAACCGTGGTACAAGGCCATG |
| | | CTAGAATAA |
| 44 | Gal | MAVEENNVPVVSQQPQAGEDVISSLSKDSHLSAQSQKYSNDELKAGESG |
| | permease | PEGSQSVPIEIPKKPMSEYVTVSLLCLCVAFGGFMFGWDTSTISGFVVQT |
| | | DFLRRFGMKHKDGTHYLSNVRTGLIVAIFNIGCAFGGIILSKGGDMYGRK |
| | | KGLSIVVSVYIVGIIIQIASINKWYQYFIGRIISGLGVGGIAVLCPMLISEIAP |
| | | KHLRGTLVSCYQLMITAGIFLGYCTNYGTKSYSNSVQWRVPLGLCFAWS |
| | | LFMIGALTLVPESPRYLCEVNKVEDAKRSIAKSNKVSPEDPAVQAELDLI |
| | | MAGIEAEKLAGNASWGELFSTKTKVFQRLLMGVFVQMFQQLTGNNYFF |
| | | YYGTVIFKSVGLDDSFETSIVIGVVNFASTFFSLWTVENLGRRKCLLLGA |
| | | ATMMACMVIYASVGVTRLYPHGKSQPSSKGAGNCMIVFTCFYIFCYATT |
| | | WAPVAWVITAESFPLRVKSKCMALASASNWVWGFLIAFFTPFITSAINFY |
| | | YGYVFMGCLVAMFFYVFFFVPETKGLSLEEIQELWEEGVLPWKSEGWIP |
| | | SSRRGNNYDLEDLQHDDKPWYKAMLE |
| 45 | DNA | ATGACAGCTCAGTTACAAAGTGAAAGTACTTCTAAAATTGTTTTGGTT |
| | encodes | ACAGGTGGTGCTGGATACATTGGTTCACACACTGTGGTAGAGCTAAT |
| | ScGAL10 | TGAGAATGGATATGACTGTGTTGTTGCTGATAACCTGTCGAATTCAA |
| | | CTTATGATTCTGTAGCCAGGTTAGAGGTCTTGACCAAGCATCACATTC |
| | | CCTTCTATGAGGTTGATTTGTGTGACCGAAAAGGTCTGGAAAAGGTT |
| | | TTCAAAGAATATAAAATTGATTCGGTAATTCACTTTGCTGGTTTAAAG |
| | | GCTGTAGGTGAATCTACACAAATCCCGCTGAGATACTATCACAATAA |
| | | CATTTTGGGAACTGTCGTTTTATTAGAGTTAATGCAACAATACAACGT |
| | | TTCCAAATTTGTTTTTTCATCTTCTGCTACTGTCTATGGTGATGCTACG |
| | | AGATTCCCAAATATGATTCCTATCCCAGAAGAATGTCCCTTAGGGCC |
| | | TACTAATCCGTATGGTCATACGAAATACGCCATTGAGAATATCTTGA |
| | | ATGATCTTTACAATAGCGACAAAAAAAGTTGGAAGTTTGCTATCTTG |
| | | CGTTATTTTAACCCAATTGGCGCACATCCCTCTGGATTAATCGGAGAA |
| | | GATCCGCTAGGTATACCAAACAATTTGTTGCCATATATGGCTCAAGT |
| | | AGCTGTTGGTAGGCGCGAGAAGCTTTACATCTTCGGAGACGATTATG |
| | | ATTCCAGAGATGGTACCCCGATCAGGGATTATATCCACGTAGTTGAT |
| | | CTAGCAAAAGGTCATATTGCAGCCCTGCAATACCTAGAGGCCTACAA |
| | | TGAAAATGAAGGTTTGTGTCGTGAGTGGAACTTGGGTTCCGGTAAAG |
| | | GTTCTACAGTTTTTGAAGTTTATCATGCATTCTGCAAAGCTTCTGGTA |
| | | TTGATCTTCCATACAAAGTTACGGGCAGAAGAGCAGGTGATGTTTTG |
| | | AACTTGACGGCTAAACCAGATAGGGCCAAACGCGAACTGAAATGGC |
| | | AGACCGAGTTGCAGGTTGAAGACTCCTGCAAGGATTTATGGAAATGG |
| | | ACTACTGAGAATCCTTTTGGTTACCAGTTAAGGGGTGTCGAGGCCAG |
| | | ATTTTCCGCTGAAGATATGCGTTATGACGCAAGATTTGTGACTATTGG |
| | | TGCCGGCACCAGATTTCAAGCCACGTTTGCCAATTTGGGCGCCAGCA |
| | | TTGTTGACCTGAAAGTGAACGGACAATCAGTTGTTCTTGGCTATGAA |
| | | AATGAGGAAGGGTATTTGAATCCTGATAGTGCTTATATAGGCGCCAC |
| | | GATCGGCAGGTATGCTAATCGTATTTCGAAGGGTAAGTTTAGTTTATG |
| | | CAACAAAGACTATCAGTTAACCGTTAATAACGGCGTTAATGCGAATC |
| | | ATAGTAGTATCGGTTCTTTCCACAGAAAAAGATTTTTGGGACCCATC |
| | | ATTCAAAATCCTTCAAAGGATGTTTTTACCGCCGAGTACATGCTGATA |
| | | GATAATGAGAAGGACACCGAATTTCCAGGTGATCTATTGGTAACCAT |
| | | ACAGTATACTGTGAACGTTGCCCAAAAAAGTTTGGAAATGGTATATA |
| | | AAGGTAAATTGACTGCTGGTGAAGCGACGCCAATAAATTTAACAAAT |
| | | CATAGTTATTTCAATCTGAACAAGCCATATGGAGACACTATTGAGGG |
| | | TACGGAGATTATGGTGCGTTCAAAAAAATCTGTTGATGTCGACAAAA |
| | | ACATGATTCCTACGGGTAATATCGTCGATAGAGAAATTGCTACCTTT |
| | | AACTCTACAAAGCCAACGGTCTTAGGCCCCAAAAATCCCCAGTTTGA |
| | | TTGTTGTTTTGTGGTGGATGAAAATGCTAAGCCAAGTCAAATCAATA |
| | | CTCTAAACAATGAATTGACGCTTATTGTCAAGGCTTTTCATCCCGATT |
| | | CCAATATTACATTAGAAGTTTTAAGTACAGAGCCAACTTATCAATTTT |
| | | ATACCGGTGATTTCTTGTCTGCTGGTTACGAAGCAAGACAAGGTTTTG |
| | | CAATTGAGCCTGGTAGATACATTGATGCTATCAATCAAGAGAACTGG |
| | | AAAGATTGTGTAACCTTGAAAAACGGTGAAACTTACGGGTCCAAGAT |
| | | TGTCTACAGATTTTCCTGA |
| 46 | ScGal10 | MTAQLQSESTSKIVLVTGGAGYIGSHTVVELIENGYDCVVADNLSNSTY |
| | | DSVARLEVLTKHHIPFYEVDLCDRKGLEKVFKEYKIDSVIHFAGLKAVGE |
| | | STQIPLRYYHNNILGTVVLLELMQQYNVSKFVFSSSATVYGDATRFPNMI |
| | | PIPEECPLGPTNPYGHTKYAIENILNDLYNSDKKSWKFAILRYFNPIGAHP |
| | | SGLIGEDPLGIPNNLLPYMAQVAVGRREKLYIFGDDYDSRDGTPIRDYIH |
| | | VVDLAKGHIAALQYLEAYNENEGLCREWNLGSGKGSTVFEVYHAFCKA |
| | | SGIDLPYKVTGRRAGDVLNLTAKPDRAKRELKWQTELQVEDSCKDLWK |
| | | WTTENPFGYQLRGVEARFSAEDMRYDARFVTIGAGTRFQATFANLG |
| | | ASIVDLKVNGQSVVLGYENEEGYLNPDSAYIGATIGRYANRISKGKFSLC |
| | | NKDYQLTVNNGVNANHSSIGSFHRKRFLGPIIQNPSKDVFTAEYMLIDNE |
| | | KDTEFPGDLLVTIQYTVNVAQKSLEMVYKGKLTAGEATPINLTNHSYFN |
| | | LNKPYGDTIEGTEIMVRSKKSVDVDKNMIPTGNIVDREIATFNSTKPTVL |
| | | GPKNPQFDCCFVVDENAKPSQINTLNNELTLIVKAFHPDSNITLEVLSTEP |
| | | TYQFYTGDFLSAGYEARQGFAIEPGRYIDAINQENWKDCVTLKNGETYG |
| | | SKIVYRFS |
| 47 | DNA | ATGGCAGAGAAGGTGCTGGTAACAGGTGGGGCTGGCTACATTGGCA |
| | encodes | GCCACACGGTGCTGGAGCTGCTGGAGGCTGGCTACTTGCCTGTGGTC |
| | human | ATCGATAACTTCCATAATGCCTTCCGTGGAGGGGGCTCCCTGCCTGA |
| | GalE | GAGCCTGCGGCGGGTCCAGGAGCTGACAGGCCGCTCTGTGGAGTTTG |
| | | AGGAGATGGACATTTTGGACCAGGGAGCCCTACAGCGTCTCTTCAAA |
| | | AAGTACAGCTTTATGGCGGTCATCCACTTTGCGGGGCTCAAGGCCG |
| | | TGGGCGAGTCGGTGCAGAAGCCTCTGGATTATTACAGAGTTAACCTG |
| | | ACCGGGACCATCCAGCTTCTGGAGATCATGAAGGCCCACGGGGTGAA |
| | | GAACCTGGTGTTCAGCAGCTCAGCCACTGTGTACGGGAACCCCCAG |
| | | TACCTGCCCCTTGATGAGGCCCACCCCACGGGTGGTTGTACCAACCC |
| | | TTACGGCAAGTCCAAGTTCTTCATCGAGGAAATGATCCGGGACCTGT |
| | | GCCAGGCAGACAAGACTTGGAACGCAGTGCTGCTGCGCTATTTCAA |
| | | CCCCACAGGTGCCCATGCCTCTGGCTGCATTGGTGAGGATCCCCAGG |
| | | GCATACCCAACAACCTCATGCCTTATGTCTCCCAGGTGGCGATCGGG |
| | | CGACGGGAGGCCCTGAATGTCTTTGGCAATGACTATGACACAGAGG |
| | | ATGGCACAGGTGTCCGGGATTACATCCATGTCGTGGATCTGGCCAAG |
| | | GGCCACATTGCAGCCTTAAGGAAGCTGAAAGAACAGTGTGGCTGCCG |
| | | GATCTACAACCTGGGCACGGGCACAGGCTATTCAGTGCTGCAGATG |
| | | GTCCAGGCTATGGAGAAGGCCTCTGGGAAGAAGATCCCGTACAAGG |
| | | TGGTGGCACGGCGGGAAGGTGATGTGGCAGCCTGTTACGCCAACCCC |
| | | AGCCTGGCCCAAGAGGAGCTGGGGTGGACAGCAGCCTTAGGGCTGG |
| | | ACAGGATGTGTGAGGATCTCTGGCGCTGGCAGAAGCAGAATCCTTCA |
| | | GGCTTTGGCACGCAAGCCTGA |
| 48 | hGalE | MAEKVLVTGGAGYIGSHTVLELLEAGYLPVVIDNFHNAFRGGGSLPESL |
| | | RRVQELTGRSVEFEEMDILDQGALQRLFKKYSFMAVIHFAGLKAVGESV |
| | | QKPLDYYRVNLTGTIQLLEIMKAHGVKNLVESSSATVYGNPQYLPLDEA |
| | | HPTGGCTNPYGKSKFFIEEMIRDLCQADKTWNVVLLRYFNPTGAHASGC |
| | | IGEDPQGIPNNLMPYVSQVAIGRREALNVFGNDYDTEDGTGVRDYIHVV |
| | | DLAKGHIAALRKLKEQCGCRIYNLGTGTGYSVLQMVQAMEKASGKKIP |
| | | YKVVARREGDVAACYANPSLAQEELGWTAALGLDRMCEDLWRWQKQ |
| | | NPSGFGTQA |
| 49 | DNA | GGCCGCGACCTGAGCCGCCTGCCCCAACTGGTCGGAGTCTCCACACC |
| | encodes | GCTGCAGGGCGGCTCGAACAGTGCCGCCGCCATCGGGCAGTCCTCCG |
| | hGalT I | GGGAGCTCCGGACCGGAGGGGCCCGGCCGCCGCCTCCTCTAGGCGCC |
| | catalytic | TCCTCCCAGCCGCGCCCGGGTGGCGACTCCAGCCCAGTCGTGGATTC |
| | domain | TGGCCCTGGCCCCGCTAGCAACTTGACCTCGGTCCCAGTGCCCCACA |
| | | CCACCGCACTGTCGCTGCCCGCCTGCCCTGAGGAGTCCCCGCTGCTT |
| | | GTGGGCCCCATGCTGATTGAGTTTAACATGCCTGTGGACCTGGAGCT |
| | | CGTGGCAAAGCAGAACCCAAATGTGAAGATGGGCGGCCGCTATGCC |
| | | CCCAGGGACTGCGTCTCTCCTCACAAGGTGGCCATCATCATTCCATTC |
| | | CGCAACCGGCAGGAGCACCTCAAGTACTGGCTATATTATTTGCACCC |
| | | AGTCCTGCAGCGCCAGCAGCTGGACTATGGCATCTATGTTATCAACC |
| | | AGGCGGGAGACACTATATTCAATCGTGCTAAGCTCCTCAATGTTGGC |
| | | TTTCAAGAAGCCTTGAAGGACTATGACTACACCTGCTTTGTGTTTAGT |
| | | GACGTGGACCTCATTCCAATGAATGACCATAATGCGTACAGGTGTTT |
| | | TTCACAGCCACGGCACATTTCCGTTGCAATGGATAAGTTTGGATTCA |
| | | GCCTACCTTATGTTCAGTATTTTGGAGGTGTCTCTGCTCTAAGTAAAC |
| | | AACAGTTTCTAACCATCAATGGATTTCCTAATAATTATTGGGGCTGGG |
| | | GAGGAGAAGATGATGACATTTTTAACAGATTAGTTTTTAGAGGCATG |
| | | TCTATATCTCGCCCAAATGCTGTGGTCGGGAGGTGTCGCATGATCCG |
| | | CCACTCAAGAGACAAGAAAAATGAACCCAATCCTCAGAGGTTTGACC |
| | | GAATTGCACACACAAAGGAGACAATGCTCTCTGATGGTTTGAACTCA |
| | | CTCACCTACCAGGTGCTGGATGTACAGAGATACCCATTGTATACCCA |
| | | AATCACAGTGGACATCGGGACACCGAGCTAG |
| 50 | hGalT I | GRDLSRLPQLVGVSTPLQGGSNSAAAIGQSSGELRTGGARPPPPLGASSQ |
| | catalytic | PRPGGDSSPVVDSGPGPASNLTSVPVPHTTALSLPACPEESPLLVGPMLIE |
| | doman | FNMPVDLELVAKQNPNVKMGGRYAPRDCVSPHKVAIIIPFRNRQEHLKY |
| | | WLYYLHPVLQRQQLDYGIYVINQAGDTIFNRAKLLNVGFQEALKDYDYT |
| | | CFVFSDVDLIPMNDHNAYRCFSQPRHISVAMDKFGFSLPYVQYFGGVSA |
| | | LSKQQFLTINGFPNNYWGWGGEDDDIFNRLVFRGMSISRPNAVVGRCR |
| | | MIRHSRDKKNEPNPQRFDRIAHTKETMLSDGLNSLTYQVLDVQRYPLYT |
| | | QITVDIGTPS |
| 51 | DNA | TCAGTCAGTGCTCTTGATGGTGACCCAGCAAGTTTGACCAGAGAAGT |
| | encodes | GATTAGATTGGCCCAAGACGCAGAGGTGGAGTTGGAGAGACAACGT |
| | human | GGACTGCTGCAGCAAATCGGAGATGCATTGTCTAGTCAAAGAGGTAG |
| | GnTI | GGTGCCTACCGCAGCTCCTCCAGCACAGCCTAGAGTGCATGTGACCC |
| | catalytic | CTGCACCAGCTGTGATTCCTATCTTGGTCATCGCCTGTGACAGATCTA |
| | doman | CTGTTAGAAGATGTCTGGACAAGCTGTTGCATTACAGACCATCTGCT |
| | Codon- | GAGTTGTTCCCTATCATCGTTAGTCAAGACTGTGGTCACGAGGAGAC |
| | optimized | TGCCCAAGCCATCGCCTCCTACGGATCTGCTGTCACTCACATCAGAC |
| | | AGCCTGACCTGTCATCTATTGCTGTGCCACCAGACCACAGAAAGTTC |
| | | CAAGGTTACTACAAGATCGCTAGACACTACAGATGGGCATTGGGTCA |
| | | AGTCTTCAGACAGTTTAGATTCCCTGCTGCTGTGGTGGTGGAGGATG |
| | | ACTTGGAGGTGGCTCCTGACTTCTTTGAGTACTTTAGAGCAACCTATC |
| | | CATTGCTGAAGGCAGACCCATCCCTGTGGTGTGTCTCTGCCTGGAAT |
| | | GACAACGGTAAGGAGCAAATGGTGGACGCTTCTAGGCCTGAGCTGTT |
| | | GTACAGAACCGACTTCTTTCCTGGTCTGGGATGGTTGCTGTTGGCTGA |
| | | GTTGTGGGCTGAGTTGGAGCCTAAGTGGCCAAAGGCATTCTGGGACG |
| | | ACTGGATGAGAAGACCTGAGCAAAGACAGGGTAGAGCCTGTATCAG |
| | | ACCTGAGATCTCAAGAACCATGACCTTTGGTAGAAAGGGAGTGTCTC |
| | | ACGGTCAATTCTTTGACCAACACTTGAAGTTTATCAAGCTGAACCAG |
| | | CAATTTGTGCACTTCACCCAACTGGACCTGTCTTACTTGCAGAGAGA |
| | | GGCCTATGACAGAGATTTCCTAGCTAGAGTCTACGGAGCTCCTCAAC |
| | | TGCAAGTGGAGAAAGTGAGGACCAATGACAGAAAGGAGTTGGGAGA |
| | | GGTGAGAGTGCAGTACACTGGTAGGGACTCCTTTAAGGCTTTCGCTA |
| | | AGGCTCTGGGTGTCATGGATGACCTTAAGTCTGGAGTTCCTAGAGCT |
| | | GGTTACAGAGGTATTGTCACCTTTCAATTCAGAGGTAGAAGAGTCCA |
| | | CTTGGCTCCTCCACCTACTTGGGAGGGTTATGATCCTTCTTGGAATTA |
| | | G |
| 52 | Human | SVSALDGDPASLTREVIRLAQDAEVELERQRGLLQQIGDALSSQRGRVPT |
| | GnT I | AAPPAQPRVHVTPAPAVIPILVIACDRSTVRRCLDKLLHYRPSAELFPIIVS |
| | catalytic | QDCGHEETAQAIASYGSAVTHIRQPDLSSIAVPPDHRKFQGYYKIARHYR |
| | doman | WALGQVFRQFRFPAAVVVEDDLEVAPDFFEYFRATYPLLKADPSLWCV |
| | | SAWNDNGKEQMVDASRPELLYRTDFFPGLGWLLLAELWAELEPKWPK |
| | | AFWDDWMRRPEQRQGRACIRPEISRTMTFGRKGVSHGQFFDQHLKFIKL |
| | | NQQFVHFTQLDLSYLQREAYDRDFLARVYGAPQLQVEKVRTNDRKELG |
| | | EVRVQYTGRDSFKAFAKALGVMDDLKSGVPRAGYRGIVTFQFRGRRVH |
| | | LAPPPTWEGYDPSWN |
| 53 | DNA | GAGCCCGCTGACGCCACCATCCGTGAGAAGAGGGCAAAGATCAAAG |
| | encodes | AGATGATGACCCATGCTTGGAATAATTATAAACGCTATGCGTGGGGC |
| | Mm ManI | TTGAACGAACTGAAACCTATATCAAAAGAAGGCCATTCAAGCAGTTT |
| | catalytic | GTTTGGCAACATCAAAGGAGCTACAATAGTAGATGCCCTGGATACCC |
| | doman | TTTTCATTATGGGCATGAAGACTGAATTTCAAGAAGCTAAATCGTGG |
| | | ATTAAAAAATATTTAGATTTTAATGTGAATGCTGAAGTTTCTGTTTTT |
| | | GAAGTCAACATACGCTTCGTCGGTGGACTGCTGTCAGCCTACTATTTG |
| | | TCCGGAGAGGAGATATTTCGAAAGAAAGCAGTGGAACTTGGGGTAA |
| | | AATTGCTACCTGCATTTCATACTCCCTCTGGAATACCTTGGGCATTGC |
| | | TGAATATGAAAAGTGGGATCGGGCGGAACTGGCCCTGGGCCTCTGGA |
| | | GGCAGCAGTATCCTGGCCGAATTTGGAACTCTGCATTTAGAGTTTAT |
| | | GCACTTGTCCCACTTATCAGGAGACCCAGTCTTTGCCGAAAAGGTTA |
| | | TGAAAATTCGAACAGTGTTGAACAAACTGGACAAACCAGAAGGCCTT |
| | | TATCCTAACTATCTGAACCCCAGTAGTGGACAGTGGGGTCAACATCA |
| | | TGTGTCGGTTGGAGGACTTGGAGACAGCTTTTATGAATATTTGCTTAA |
| | | GGCGTGGTTAATGTCTGACAAGACAGATCTCGAAGCCAAGAAGATGT |
| | | ATTTTGATGCTGTTCAGGCCATCGAGACTCACTTGATCCGCAAGTCAA |
| | | GTGGGGGACTAACGTACATCGCAGAGTGGAAGGGGGGCCTCCTGGA |
| | | ACACAAGATGGGCCACCTGACGTGCTTTGCAGGAGGCATGTTTGCAC |
| | | TTGGGGCAGATGGAGCTCCGGAAGCCCGGGCCCAACACTACCTTGAA |
| | | CTCGGAGCTGAAATTGCCCGCACTTGTCATGAATCTTATAATCGTACA |
| | | TATGTGAAGTTGGGACCGGAAGCGTTTCGATTTGATGGCGGTGTGGA |
| | | AGCTATTGCCACGAGGCAAAATGAAAAGTATTACATCTTACGGCCCG |
| | | AGGTCATCGAGACATACATGTACATGTGGCGACTGACTCACGACCCC |
| | | AAGTACAGGACCTGGGCCTGGGAAGCCGTGGAGGCTCTAGAAAGTC |
| | | ACTGCAGAGTGAACGGAGGCTACTCAGGCTTACGGGATGTTTACATT |
| | | GCCCGTGAGAGTTATGACGATGTCCAGCAAAGTTTCTTCCTGGCAGA |
| | | GACACTGAAGTATTTGTACTTGATATTTTCCGATGATGACCTTCTTCC |
| | | ACTAGAACACTGGATCTTCAACACCGAGGCTCATCCTTTCCCTATACT |
| | | CCGTGAACAGAAGAAGGAAATTGATGGCAAAGAGAAATGA |
| 54 | Mm ManI | EPADATIREKRAKIKEMMTHAWNNYKRYAWGLNELKPISKEGHSSSLFG |
| | catalytic | NIKGATIVDALDTLFIMGMKTEFQEAKSWIKKYLDFNVNAEVSVFEVNIR |
| | doman | FVGGLLSAYYLSGEEIFRKKAVELGVKLLPAFHTPSGIPWALLNMKSGIG |
| | | RNWPWASGGSSILAEFGTLHLEFMHLSHLSGDPVFAEKVMKIRTVLNKL |
| | | DKPEGLYPNYLNPSSGQWGQHHVSVGGLGDSFYEYLLKAWLMSDKTD |
| | | LEAKKMYFDAVQATETHLIRKSSGGLTYIAEWKGGLLEHKMGHLTCFAG |
| | | GMFALGADGAPEARAQHYLELGAEIARTCHESYNRTYVKLGPEAFRFD |
| | | GGVEAIATRQNEKYYILRPEVIETYMYMWRLTHDPKYRTWAWEAVEAL |
| | | ESHCRVNGGYSGLRDVYIARESYDDVQQSFFLAETLKYLYLIFSDDDLLP |
| | | LEHWIFNTEAHPFPILREQKKEIDGKEK |
| 55 | DNA | CGCGCCGGATCTCCCAACCCTACGAGGGCGGCAGCAGTCAAGGCCG |
| | encodes Tr | CATTCCAGACGTCGTGGAACGCTTACCACCATTTTGCCTTTCCCCATG |
| | ManI | ACGACCTCCACCCGGTCAGCAACAGCTTTGATGATGAGAGAAACGGC |
| | catalytic | TGGGGCTCGTCGGCAATCGATGGCTTGGACACGGCTATCCTCATGGG |
| | doman | GGATGCCGACATTGTGAACACGATCCTTCAGTATGTACCGCAGATCA |
| | | ACTTCACCACGACTGCGGTTGCCAACCAAGGCATCTCCGTGTTCGAG |
| | | ACCAACATTCGGTACCTCGGTGGCCTGCTTTCTGCCTATGACCTGTTG |
| | | CGAGGTCCTTTCAGCTCCTTGGCGACAAACCAGACCCTGGTAAACAG |
| | | CCTTCTGAGGCAGGCTCAAACACTGGCCAACGGCCTCAAGGTTGCGT |
| | | TCACCACTCCCAGCGGTGTCCCGGACCCTACCGTCTTCTTCAACCCTA |
| | | CTGTCCGGAGAAGTGGTGCATCTAGCAACAACGTCGCTGAAATTGGA |
| | | AGCCTGGTGCTCGAGTGGACACGGTTGAGCGACCTGACGGGAAACCC |
| | | GCAGTATGCCCAGCTTGCGCAGAAGGGCGAGTCGTATCTCCTGAATC |
| | | CAAAGGGAAGCCCGGAGGCATGGCCTGGCCTGATTGGAACGTTTGTC |
| | | AGCACGAGCAACGGTACCTTTCAGGATAGCAGCGGCAGCTGGTCCGG |
| | | CCTCATGGACAGCTTCTACGAGTACCTGATCAAGATGTACCTGTACG |
| | | ACCCGGTTGCGTTTGCACACTACAAGGATCGCTGGGTCCTTGCTGCC |
| | | GACTCGACCATTGCGCATCTCGCCTCTCACCCGTCGACGCGCAAGGA |
| | | CTTGACCTTTTTGTCTTCGTACAACGGACAGTCTACGTCGCCAAACTC |
| | | AGGACATTTGGCCAGTTTTGCCGGTGGCAACTTCATCTTGGGAGGCA |
| | | TTCTCCTGAACGAGCAAAAGTACATTGACTTTGGAATCAAGCTTGCC |
| | | AGCTCGTACTTTGCCACGTACAACCAGACGGCTTCTGGAATCGGCCC |
| | | CGAAGGCTTCGCGTGGGTGGACAGCGTGACGGGCGCCGGCGGCTCG |
| | | CCGCCCTCGTCCCAGTCCGGGTTCTACTCGTCGGCAGGATTCTGGGTG |
| | | ACGGCACCGTATTACATCCTGCGGCCGGAGACGCTGGAGAGCTTGTA |
| | | CTACGCATACCGCGTCACGGGCGACTCCAAGTGGCAGGACCTGGCGT |
| | | GGGAAGCGTTCAGTGCCATTGAGGACGCATGCCGCGCCGGCAGCGC |
| | | GTACTCGTCCATCAACGACGTGACGCAGGCCAACGGCGGGGGTGCCT |
| | | CTGACGATATGGAGAGCTTCTGGTTTGCCGAGGCGCTCAAGTATGCG |
| | | TACCTGATCTTTGCGGAGGAGTCGGATGTGCAGGTGCAGGCCAACGG |
| | | CGGGAACAAATTTGTCTTTAACACGGAGGCGCACCCCTTTAGCATCC |
| | | GTTCATCATCACGACGGGGCGGCCACCTTGCTTAA |
| 56 | Tr Man I | RAGSPNPTRAAAVKAAFQTSWNAYHHFAFPHDDLHPVSNSFDDERNG |
| | catalytic | WGSSAIDGLDTAILMGDADIVNTILQYVPQINFTTTAVANQGISVFETNIR |
| | Boman | YLGGLLSAYDLLRGPFSSLATNQTLVNSLLRQAQTLANGLKVAFTTPSG |
| | | VPDPTVFFNPTVRRSGASSNNVAEIGSLVLEWTRLSDLTGNPQYAQLAQ |
| | | KGESYLLNPKGSPEAWPGLIGTFVSTSNGTFQDSSGSWSGLMDSFYEYLI |
| | | KMYLYDPVAFAHYKDRWVLAADSTIAHLASHPSTRKDLTFLSSYNGQS |
| | | TSPNSGHLASFAGGNFILGGILLNEQKYIDFGIKLASSYFATYNQTASGIGP |
| | | EGFAWVDSVTGAGGSPPSSQSGFYSSAGFWVTAPYYILRPETLESLYYA |
| | | YRVTGDSKWQDLAWEAFSAIEDACRAGSAYSSINDVTQANGGGASDD |
| | | MESFWFAEALKYAYLIFAEESDVQVQANGGNKFVFNTEAHPFSIRSSSRR |
| | | GGHLA |
| 57 | DNA | TCCCTAGTGTACCAGTTGAACTTTGATCAGATGCTGAGGAATGTCGA |
| | encodes | TAAAGACGGCACCTGGAGTCCGGGGGAGCTGGTGCTGGTGGTCCAA |
| | Rat GnTII | GTGCATAACAGGCCGGAATACCTCAGGCTGCTGATAGACTCGCTTCG |
| | DNA | AAAAGCCCAGGGTATTCGCGAAGTCCTAGTCATCTTTAGCCATGACT |
| | (TC) | TCTGGTCGGCAGAGATCAACAGTCTGATCTCTAGTGTGGACTTCTGTC |
| | | CGGTTCTGCAAGTGTTCTTTCCGTTCAGCATTCAGCTGTACCCGAGTG |
| | | AGTTTCCGGGTAGTGATCCCAGAGATTGCCCCAGAGACCTGAAGAAG |
| | | AATGCAGCTCTCAAGTTGGGGTGCATCAATGCCGAATACCCAGACTC |
| | | CTTCGGCCATTACAGAGAGGCCAAATTCTCGCAAACCAAACATCACT |
| | | GGTGGTGGAAGCTGCATTTTGTATGGGAAAGAGTCAAAGTTCTTCAA |
| | | GATTACACTGGCCTTATACTTTTCCTGGAAGAGGACCACTACTTAGCC |
| | | CCAGACTTTTACCATGTCTTCAAAAAGATGTGGAAATTGAAGCAGCA |
| | | GGAGTGTCCTGGGTGTGACGTCCTCTCTCTAGGGACCTACACCACCA |
| | | TTCGGAGTTTCTATGGTATTGCTGACAAAGTAGATGTGAAAACTTGG |
| | | AAATCGACAGAGCACAATATGGGGCTAGCCTTGACCCGAGATGCATA |
| | | TCAGAAGCTTATCGAGTGCACGGACACTTTCTGTACTTACGATGATTA |
| | | TAACTGGGACTGGACTCTTCAATATTTGACTCTAGCTTGTCTTCCTAA |
| | | AGTCTGGAAAGTCTTAGTTCCTCAAGCTCCTAGGATTTTTCATGCTGG |
| | | AGACTGTGGTATGCATCACAAGAAAACATGTAGGCCATCCACCCAGA |
| | | GTGCCCAAATTGAGTCATTATTAAATAATAATAAACAGTACCTGTTTC |
| | | CAGAAACTCTAGTTATCGGTGAGAAGTTTCCTATGGCAGCCATTTCCC |
| | | CACCTAGGAAAAATGGAGGGTGGGGAGATATTAGGGACCATGAACT |
| | | CTGTAAAAGTTATAGAAGACTGCAGTGA |
| 58 | Rat GnTII | SLVYQLNFDQMLRNVDKDGTWSPGELVLVVQVHNRPEYLRLLIDSLRK |
| | (TC) | AQGIREVLVIFSHDFWSAEINSLISSVDFCPVLQVFFPFSIQLYPSEFPGSDP |
| | | RDCPRDLKKNAALKLGCINAEYPDSFGHYREAKFSQTKHHWWWKLHF |
| | | VWERVKVLQDYTGLILFLEEDHYLAPDFYHVFKKMWKLKQQECPGCD |
| | | VLSLGTYTTIRSFYGIADKVDVKTWKSTEHNMGLALTRDAYQKLIECTD |
| | | TFCTYDDYNWDWTLQYLTLACLPKVWKVLVPQAPRIFHAGDCGMHHK |
| | | KTCRPSTQSAQIESLLNNNKQYLFPETLVIGEKFPMAAISPPRKNGGWGDI |
| | | RDHELCKSYRRLQ |
| 59 | DNA | TCCTTGGTTTACCAATTGAACTTCGACCAGATGTTGAGAAACGTTGAC |
| | encodes | AAGGACGGTACTTGGTCTCCTGGTGAGTTGGTTTTGGTTGTTCAGGTT |
| | Rat GnT II | CACAACAGACCAGAGTACTTGAGATTGTTGATCGACTCCTTGAGAAA |
| | (TC) | GGCTCAAGGTATCAGAGAGGTTTTGGTTATCTTCTCCCACGATTTCTG |
| | Codon- | GTCTGCTGAGATCAACTCCTTGATCTCCTCCGTTGACTTCTGTCCAGT |
| | optimized | TTTGCAGGTTTTCTTCCCATTCTCCATCCAATTGTACCCATCTGAGTTC |
| | | CCAGGTTCTGATCCAAGAGACTGTCCAAGAGACTTGAAGAAGAACGC |
| | | TGCTTTGAAGTTGGGTTGTATCAACGCTGAATACCCAGATTCTTTCGG |
| | | TCACTACAGAGAGGCTAAGTTCTCCCAAACTAAGCATCATTGGTGGT |
| | | GGAAGTTGCACTTTGTTTGGGAGAGAGTTAAGGTTTTGCAGGACTAC |
| | | ACTGGATTGATCTTGTTCTTGGAGGAGGATCATTACTTGGCTCCAGAC |
| | | TTCTACCACGTTTTCAAGAAGATGTGGAAGTTGAAGCAACAAGAGTG |
| | | TCCAGGTTGTGACGTTTTGTCCTTGGGAACTTACACTACTATCAGATC |
| | | CTTCTACGGTATCGCTGACAAGGTTGACGTTAAGACTTGGAAGTCCA |
| | | CTGAACACAACATGGGATTGGCTTTGACTAGAGATGCTTACCAGAAG |
| | | TTGATCGAGTGTACTGACACTTTCTGTACTTACGACGACTACAACTGG |
| | | GACTGGACTTTGCAGTACTTGACTTTGGCTTGTTTGCCAAAAGTTTGG |
| | | AAGGTTTTGGTTCCACAGGCTCCAAGAATTTTCCACGCTGGTGACTGT |
| | | GGAATGCACCACAAGAAAACTTGTAGACCATCCACTCAGTCCGCTCA |
| | | AATTGAGTCCTTGTTGAACAACAACAAGCAGTACTTGTTCCCAGAGA |
| | | CTTTGGTTATCGGAGAGAAGTTTCCAATGGCTGCTATTTCCCCACCAA |
| | | GAAAGAATGGTGGATGGGGTGATATTAGAGACCACGAGTTGTGTAA |
| | | ATCCTACAGAAGATTGCAGTAG |
| 60 | DNA | AGGAAGAACGACGCCCTTGCCCCGCCGCTGCTGGACTCGGAGCCCCT |
| | encodes | ACGGGGTGCGGGCCATTTCGCCGCGTCCGTAGGCATCCGCAGGGTTT |
| | Rat GnTII | CTAACGACTCGGCCGCTCCTCTGGTTCCCGCGGTCCCGCGGCCGGAG |
| | (TA) | GTGGACAACCTAACGCTGCGGTACCGGTCCCTAGTGTACCAGTTGAA |
| | | CTTTGATCAGATGCTGAGGAATGTCGATAAAGACGGCACCTGGAGTC |
| | | CGGGGGAGCTGGTGCTGGTGGTCCAAGTGCATAACAGGCCGGAATA |
| | | CCTCAGGCTGCTGATAGACTCGCTTCGAAAAGCCCAGGGTATTCGCG |
| | | AAGTCCTAGTCATCTTTAGCCATGACTTCTGGTCGGCAGAGATCAAC |
| | | AGTCTGATCTCTAGTGTGGACTTCTGTCCGGTTCTGCAAGTGTTCTTT |
| | | CCGTTCAGCATTCAGCTGTACCCGAGTGAGTTTCCGGGTAGTGATCCC |
| | | AGAGATTGCCCCAGAGACCTGAAGAAGAATGCAGCTCTCAAGTTGG |
| | | GGTGCATCAATGCCGAATACCCAGACTCCTTCGGCCATTACAGAGAG |
| | | GCCAAATTCTCGCAAACCAAACATCACTGGTGGTGGAAGCTGCATTT |
| | | TGTATGGGAAAGAGTCAAAGTTCTTCAAGATTACACTGGCCTTATAC |
| | | TTTTCCTGGAAGAGGACCACTACTTAGCCCCAGACTTTTACCATGTCT |
| | | TCAAAAAGATGTGGAAATTGAAGCAGCAGGAGTGTCCTGGGTGTGAC |
| | | GTCCTCTCTCTAGGGACCTACACCACCATTCGGAGTTTCTATGGTAT |
| | | TGCTGACAAAGTAGATGTGAAAACTTGGAAATCGACAGAGCACAAT |
| | | ATGGGGCTAGCCTTGACCCGAGATGCATATCAGAAGCTTATCGAGTG |
| | | CACGGACACTTTCTGTACTTACGATGATTATAACTGGGACTGGACTCT |
| | | TCAATATTTGACTCTAGCTTGTCTTCCTAAAGTCTGGAAAGTCTTAGT |
| | | TCCTCAAGCTCCTAGGATTTTTCATGCTGGAGACTGTGGTATGCATCA |
| | | CAAGAAAACATGTAGGCCATCCACCCAGAGTGCCCAAATTGAGTCAT |
| | | TATTAAATAATAATAAACAGTACCTGTTTCCAGAAACTCTAGTTATCG |
| | | GTGAGAAGTTTCCTATGGCAGCCATTTCCCCACCTAGGAAAAATGGA |
| | | GGGTGGGGAGATATTAGGGACCATGAACTCTGTAAAAGTTATAGAAG |
| | | ACTGCAGTGAGtta |
| 61 | Rat GnTII | RKNDALAPPLLDSEPLRGAGHFAASVGIRRVSNDSAAPLVPAVPRPEVD |
| | (TA) | NLTLRYRSLVYQLNFDQMLRNVDKDGTWSPGELVLVVQVHNRPEYLRL |
| | | LIDSLRKAQGIREVLVIFSHDFWSAEINSLISSVDFCPVLQVFFPFSIQLYPS |
| | | EFPGSDPRDCPRDLKKNAALKLGCINAEYPDSFGHYREAKFSQTKHHW |
| | | WWKLHFVWERVKVLQDYTGLILFLEEDHYLAPDFYHVFKKMWKLKQQ |
| | | ECPGCDVLSLGTYTTIRSFYGIADKVDVKTWKSTEHNMGLALTRDAYQ |
| | | KLIECTDTFCTYDDYNWDWTLQYLTLACLPKVWKVLVPQAPRIFHAGD |
| | | CGMHHKKTCRPSTQSAQIESLLNNNKQYLFPETLVIGEKFPMAAISPPRK |
| | | NGGWGDIRDHELCKSYRRLQ |
| 62 | DNA | CGCGACGATCCAATAAGACCTCCACTTAAAGTGGCTCGTTCCCCGAG |
| | encodes | GCCAGGGCAATGCCAAGATGTGGTCCAAGACGTGCCCAATGTGGATG |
| | Dm | TACAGATGCTGGAGCTATACGATCGCATGTCCTTCAAGGACATAGAT |
| | ManII | GGAGGCGTGTGGAAACAGGGCTGGAACATTAAGTACGATCCACTGA |
| | catalytic | AGTACAACGCCCATCACAAACTAAAAGTCTTCGTTGTGCCGCACTCG |
| | doman | CACAACGATCCTGGATGGATTCAGACGTTTGAGGAATACTACCAGCA |
| | (KD) | CGACACCAAGCACATCCTGTCCAATGCACTACGGCATCTGCACGACA |
| | | ATCCCGAGATGAAGTTCATCTGGGCGGAAATCTCCTACTTTGCTCGGT |
| | | TCTATCACGATTTGGGAGAGAACAAAAAGCTGCAGATGAAGTCCATT |
| | | GTAAAGAATGGACAGTTGGAATTTGTGACTGGAGGATGGGTAATGCC |
| | | GGACGAGGCCAACTCCCACTGGCGAAACGTACTGCTGCAGCTGACCG |
| | | AAGGGCAAACATGGTTGAAGCAATTCATGAATGTCACACCCACTGCT |
| | | TCCTGGGCCATCGATCCCTTCGGACACAGTCCCACTATGCCGTACATT |
| | | TTGCAGAAGAGTGGTTTCAAGAATATGCTTATCCAAAGGACGCACTA |
| | | TTCGGTTAAGAAGGAACTGGCCCAACAGCGACAGCTTGAGTTCCTGT |
| | | GGCGCCAGATCTGGGACAACAAAGGGGACACAGCTCTCTTCACCCAC |
| | | ATGATGCCCTTCTACTCGTACGACATTCCTCATACCTGTGGTCCAGAT |
| | | CCCAAGGTTTGCTGTCAGTTCGATTTCAAACGAATGGGCTCCTTCGGT |
| | | TTGAGTTGTCCATGGAAGGTGCCGCCGCGTACAATCAGTGATCAAAA |
| | | TGTGGCAGCACGCTCAGATCTGCTGGTTGATCAGTGGAAGAAGAAGG |
| | | CCGAGCTGTATCGCACAAACGTGCTGCTGATTCCGTTGGGTGACGAC |
| | | TTCCGCTTCAAGCAGAACACCGAGTGGGATGTGCAGCGCGTGAACTA |
| | | CGAAAGGCTGTTCGAACACATCAACAGCCAGGCCCACTTCAATGTCC |
| | | AGGCGCAGTTCGGCACACTGCAGGAATACTTTGATGCAGTGCACCAG |
| | | GCGGAAAGGGCGGGACAAGCCGAGTTTCCCACGCTAAGCGGTGACT |
| | | TTTTCACATACGCCGATCGATCGGATAACTATTGGAGTGGCTACTAC |
| | | ACATCCCGCCCGTATCATAAGCGCATGGACCGCGTCCTGATGCACTA |
| | | TGTACGTGCAGCAGAAATGCTTTCCGCCTGGCACTCCTGGGACGGTA |
| | | TGGCCCGCATCGAGGAACGTCTGGAGCAGGCCCGCAGGGAGCTGTC |
| | | ATTGTTCCAGCACCACGACGGTATAACTGGCACAGCAAAAACGCACG |
| | | TAGTCGTCGACTACGAGCAACGCATGCAGGAAGCTTTAAAAGCCTGT |
| | | CAAATGGTAATGCAACAGTCGGTCTACCGATTGCTGACAAAGCCCTC |
| | | CATCTACAGTCCGGACTTCAGTTTCTCGTACTTTACGCTCGACGACTC |
| | | CCGCTGGCCAGGATCTGGTGTGGAGGACAGTCGAACCACCATAATAC |
| | | TGGGCGAGGATATACTGCCCTCCAAGCATGTGGTGATGCACAACACC |
| | | CTGCCCCACTGGCGGGAGCAGCTGGTGGACTTTTATGTATCCAGTCC |
| | | GTTTGTAAGCGTTACCGACTTGGCAAACAATCCGGTGGAGGCTCAGG |
| | | TGTCCCCGGTGTGGAGCTGGCACCACGACACACTCACAAAGACTATC |
| | | CACCCACAAGGCTCCACCACCAAGTACCGCATCATCTTCAAGGCTCG |
| | | GGTGCCGCCCATGGGCTTGGCCACCTACGTTTTAACCATCTCCGATTC |
| | | CAAGCCAGAGCACACCTCGTATGCATCGAATCTCTTGCTCCGTAAAA |
| | | ACCCGACTTCGTTACCATTGGGCCAATATCCGGAGGATGTGAAGTTT |
| | | GGCGATCCTCGAGAGATCTCATTGCGGGTTGGTAACGGACCCACCTT |
| | | GGCCTTTTCGGAGCAGGGTCTCCTTAAGTCCATTCAGCTTACTCAGGA |
| | | TAGCCCACATGTACCGGTGCACTTCAAGTTCCTCAAGTATGGCGTTCG |
| | | ATCGCATGGCGATAGATCCGGTGCCTATCTGTTCCTGCCCAATGGAC |
| | | CAGCTTCGCCAGTCGAGCTTGGCCAGCCAGTGGTCCTGGTGACTAAG |
| | | GGCAAACTGGAGTCGTCCGTGAGCGTGGGACTTCCGAGCGTGGTGCA |
| | | CCAGACGATAATGCGCGGTGGTGCACCTGAGATTCGCAATCTGGTGG |
| | | ATATAGGCTCACTGGACAACACGGAGATCGTGATGCGCTTGGAGACG |
| | | CATATCGACAGCGGCGATATCTTCTACACGGATCTCAATGGATTGCA |
| | | ATTTATCAAGAGGCGGCGTTTGGACAAATTACCTTTGCAGGCCAACT |
| | | ATTATCCCATACCTTCTGGTATGTTCATTGAGGATGCCAATACGCGAC |
| | | TCACTCTCCTCACGGGTCAACCGCTGGGTGGATCTTCTCTGGCCTCGG |
| | | GCGAGCTAGAGATTATGCAAGATCGTCGCCTGGCCAGCGATGATGAA |
| | | CGCGGCCTGGGACAGGGTGTTTTGGACAACAAGCCGGTGCTGCATAT |
| | | TTATCGGCTGGTGCTGGAGAAGGTTAACAACTGTGTCCGACCGTCAA |
| | | AGCTTCATCCTGCCGGCTATTTGACAAGTGCCGCACACAAAGCATCG |
| | | CAGTCACTGCTGGATCCACTGGACAAGTTTATATTCGCTGAAAATGA |
| | | GTGGATCGGGGCACAGGGGCAATTTGGTGGCGATCATCCTTCGGCTC |
| | | GTGAGGATCTCGATGTGTCGGTGATGAGACGCTTAACCAAGAGCTCG |
| | | GCCAAAACCCAGCGAGTAGGCTACGTTCTGCACCGCACCAATCTGAT |
| | | GCAATGCGGCACTCCAGAGGAGCATACACAGAAGCTGGATGTGTGC |
| | | CACCTACTGCCGAATGTGGCGAGATGCGAGCGCACGACGCTGACTTT |
| | | CCTGCAGAATTTGGAGCACTTGGATGGCATGGTGGCGCCGGAAGTGT |
| | | GCCCCATGGAAACCGCCGCTTATGTGAGCAGTCACTCAAGCTGA |
| 63 | Dm | RDDPIRPPLKVARSPRPGQCQDVVQDVPNVDVQMLELYDRMSFKDIDG |
| | ManII | GVWKQGWNIKYDPLKYNAHHKLKVFVVPHSHNDPGWIQTFEEYYQHD |
| | catalytic | TKHILSNALRHLHDNPEMKFIWAEISYFARFYHDLGENKKLQMKSIVKN |
| | doman | GQLEFVTGGWVMPDEANSHWRNVLLQLTEGQTWLKQFMNVTPTASW |
| | (KD) | AIDPFGHSPTMPYILQKSGFKNMLIQRTHYSVKKELAQQRQLEFLWRQI |
| | | WDNKGDTALFTHMMPFYSYDIPHTCGPDPKVCCQFDFKRMGSFGLSCP |
| | | WKVPPRTISDQNVAARSDLLVDQWKKKAELYRTNVLLIPLGDDFRFKQ |
| | | NTEWDVQRVNYERLFEHINSQAHFNVQAQFGTLQEYFDAVHQAERAGQ |
| | | AEFPTLSGDFFTYADRSDNYWSGYYTSRPYHKRMDRVLMHYVRAAEM |
| | | LSAWHSWDGMARIEERLEQARRELSLFQHHDGITGTAKTHVVVDYEQR |
| | | MQEALKACQMVMQQSVYRLLTKPSIYSPDFSFSYFTLDDSRWPGSGVED |
| | | SRTTIILGEDILPSKHVVMHNTLPHWREQLVDFYVSSPFVSVTDLANNPV |
| | | EAQVSPVWSWHHDTLTKTIHPQGSTTKYRIIFKARVPPMGLATYVLTISD |
| | | SKPEHTSYASNLLLRKNPTSLPLGQYPEDVKFGDPREISLRVGNGPTLAFS |
| | | EQGLLKSIQLTQDSPHVPVHFKFLKYGVRSHGDRSGAYLFLPNGPASPVE |
| | | LGQPVVLVTKGKLESSVSVGLPSVVHQTIMRGGAPEIRNLVDIGSLDNTEI |
| | | VMRLETHIDSGDIFYTDLNGLQFIKRRRLDKLPLQANYYPIPSGMFIEDAN |
| | | TRLTLLTGQPLGGSSLASGELEIMQDRRLASDDERGLGQGVLDNKPVLHI |
| | | YRLVLEKVNNCVRPSKLHPAGYLTSAAHKASQSLLDPLDKFIFAENEWI |
| | | GAQGQFGGDHPSAREDLDVSVMRRLTKSSAKTQRVGYVLHRTNLMQC |
| | | GTPEEHTQKLDVCHLLPNVARCERTTLTFLQNLEHLDGMVAPEVCPMET |
| | | AAYVSSHSS |
| 64 | DNA | AGAGACGATCCAATTAGACCTCCATTGAAGGTTGCTAGATCCCCAAG |
| | encodes | ACCAGGTCAATGTCAAGATGTTGTTCAGGACGTCCCAAACGTTGATG |
| | Dm ManII | TCCAGATGTTGGAGTTGTACGATAGAATGTCCTTCAAGGACATTGAT |
| | codon- | GGTGGTGTTTGGAAGCAGGGTTGGAACATTAAGTACGATCCATTGAA |
| | optimized | GTACAACGCTCATCACAAGTTGAAGGTCTTCGTTGTCCCACACTCCCA |
| | (KD) | CAACGATCCTGGTTGGATTCAGACCTTCGAGGAATACTACCAGCACG |
| | | ACACCAAGCACATCTTGTCCAACGCTTTGAGACATTTGCACGACAAC |
| | | CCAGAGATGAAGTTCATCTGGGCTGAAATCTCCTACTTCGCTAGATTC |
| | | TACCACGATTTGGGTGAGAACAAGAAGTTGCAGATGAAGTCCATCGT |
| | | CAAGAACGGTCAGTTGGAATTCGTCACTGGTGGATGGGTCATGCCAG |
| | | ACGAGGCTAACTCCCACTGGAGAAACGTTTTGTTGCAGTTGACCGAA |
| | | GGTCAAACTTGGTTGAAGCAATTCATGAACGTCACTCCAACTGCTTC |
| | | CTGGGCTATCGATCCATTCGGACACTCTCCAACTATGCCATACATTTT |
| | | GCAGAAGTCTGGTTTCAAGAATATGTTGATCCAGAGAACCCACTACT |
| | | CCGTTAAGAAGGAGTTGGCTCAACAGAGACAGTTGGAGTTCTTGTGG |
| | | AGACAGATCTGGGACAACAAAGGTGACACTGCTTTGTTCACCCACAT |
| | | GATGCCATTCTACTCTTACGACATTCCTCATACCTGTGGTCCAGATCC |
| | | AAAGGTTTGTTGTCAGTTCGATTTCAAAAGAATGGGTTCCTTCGGTTT |
| | | GTCTTGTCCATGGAAGGTTCCACCTAGAACTATCTCTGATCAAAATGT |
| | | TGCTGCTAGATCCGATTTGTTGGTTGATCAGTGGAAGAAGAAGGCTG |
| | | AGTTGTACAGAACCAACGTCTTGTTGATTCCATTGGGTGACGACTTCA |
| | | GATTCAAGCAGAACACCGAGTGGGATGTTCAGAGAGTCAACTACGA |
| | | AAGATTGTTCGAACACATCAACTCTCAGGCTCACTTCAATGTCCAGG |
| | | CTCAGTTCGGTACTTTGCAGGAATACTTCGATGCTGTTCACCAGGCTG |
| | | AAAGAGCTGGACAAGCTGAGTTCCCAACCTTGTCTGGTGACTTCTTC |
| | | ACTTACGCTGATAGATCTGATAACTACTGGTCTGGTTACTACACTTCC |
| | | AGACCATACCATAAGAGAATGGACAGAGTCTTGATGCACTACGTTAG |
| | | AGCTGCTGAAATGTTGTCCGCTTGGCACTCCTGGGACGGTATGGCTA |
| | | GAATCGAGGAAAGATTGGAGCAGGCTAGAAGAGAGTTGTCCTTGTTC |
| | | CAGCACCACGACGGTATTACTGGTACTGCTAAAACTCACGTTGTCGT |
| | | CGACTACGAGCAAAGAATGCAGGAAGCTTTGAAAGCTTGTCAAATG |
| | | GTCATGCAACAGTCTGTCTACAGATTGTTGACTAAGCCATCCATCTAC |
| | | TCTCCAGACTTCTCCTTCTCCTACTTCACTTTGGACGACTCCAGATGG |
| | | CCAGGTTCTGGTGTTGAGGACTCTAGAACTACCATCATCTTGGGTGA |
| | | GGATATCTTGCCATCCAAGCATGTTGTCATGCACAACACCTTGCCAC |
| | | ACTGGAGAGAGCAGTTGGTTGACTTCTACGTCTCCTCTCCATTCGTTT |
| | | CTGTTACCGACTTGGCTAACAATCCAGTTGAGGCTCAGGTTTCTCCAG |
| | | TTTGGTCTTGGCACCACGACACTTTGACTAAGACTATCCACCCACAA |
| | | GGTTCCACCACCAAGTACAGAATCATCTTCAAGGCTAGAGTTCCACC |
| | | AATGGGTTTGGCTACCTACGTTTTGACCATCTCCGATTCCAAGCCAGA |
| | | GCACACCTCCTACGCTTCCAATTTGTTGCTTAGAAAGAACCCAACTTC |
| | | CTTGCCATTGGGTCAATACCCAGAGGATGTCAAGTTCGGTGATCCAA |
| | | GAGAGATCTCCTTGAGAGTTGGTAACGGTCCAACCTTGGCTTTCTCTG |
| | | AGCAGGGTTTGTTGAAGTCCATTCAGTTGACTCAGGATTCTCCACATG |
| | | TTCCAGTTCACTTCAAGTTCTTGAAGTACGGTGTTAGATCTCATGGTG |
| | | ATAGATCTGGTGCTTACTTGTTCTTGCCAAATGGTCCAGCTTCTCCAG |
| | | TCGAGTTGGGTCAGCCAGTTGTCTTGGTCACTAAGGGTAAATTGGAG |
| | | TCTTCCGTTTCTGTTGGTTTGCCATCTGTCGTTCACCAGACCATCATG |
| | | AGAGGTGGTGCTCCAGAGATTAGAAATTTGGTCGATATTGGTTCTTTG |
| | | GACAACACTGAGATCGTCATGAGATTGGAGACTCATATCGACTCTGG |
| | | TGATATCTTCTACACTGATTTGAATGGATTGCAATTCATCAAGAGGA |
| | | GAAGATTGGACAAGTTGCCATTGCAGGCTAACTACTACCCAATTCCA |
| | | TCTGGTATGTTCATTGAGGATGCTAATACCAGATTGACTTTGTTGACC |
| | | GGTCAACCATTGGGTGGATCTTCTTTGGCTTCTGGTGAGTTGGAGATT |
| | | ATGCAAGATAGAAGATTGGCTTCTGATGATGAAAGAGGTTTGGGTCA |
| | | GGGTGTTTTGGACAACAAGCCAGTTTTGCATATTTACAGATTGGTCTT |
| | | GGAGAAGGTTAACAACTGTGTCAGACCATCTAAGTTGCATCCAGCTG |
| | | GTTACTTGACTTCTGCTGCTCACAAAGCTTCTCAGTCTTTGTTGGATC |
| | | CATTGGACAAGTTCATCTTCGCTGAAAATGAGTGGATCGGTGCTCAG |
| | | GGTCAATTCGGTGGTGATCATCCATCTGCTAGAGAGGATTTGGATGT |
| | | CTCTGTCATGAGAAGATTGACCAAGTCTTCTGCTAAAACCCAGAGAG |
| | | TTGGTTACGTTTTGCACAGAACCAATTTGATGCAATGTGGTACTCCAG |
| | | AGGAGCATACTCAGAAGTTGGATGTCTGTCACTTGTTGCCAAATGTT |
| | | GCTAGATGTGAGAGAACTACCTTGACTTTCTTGCAGAATTTGGAGCA |
| | | CTTGGATGGTATGGTTGCTCCAGAAGTTTGTCCAATGGAAACCGCTG |
| | | CTTACGTCTCTTCTCACTCTTCTTGA |
| 65 | DNA | GCTGAACCAAAATCTTGTGATAAAACTCATACATGTCCACCATGTCC |
| | encodes | AGCTCCTGAACTTCTGGGTGGACCATCAGTTTTCTTGTTCCCACCAAA |
| | human Fc | ACCAAAGGATACCCTTATGATTTCTAGAACTCCTGAAGTCACATGTG |
| | | TTGTTGTTGATGTTTCTCATGAAGATCCTGAAGTCAAGTTCAACTGGT |
| | | ACGTTGATGGTGTTGAAGTTCATAATGCTAAGACAAAGCCAAGAGAA |
| | | GAACAATACAACTCTACTTACAGAGTTGTCTCTGTTCTTACTGTTCTG |
| | | CATCAAGATTGGCTGAATGGTAAGGAATACAAGTGTAAGGTCTCCAA |
| | | CAAAGCTCTTCCAGCTCCAATTGAGAAAACCATTTCCAAAGCTAAAG |
| | | GTCAACCAAGAGAACCACAAGTTTACACCTTGCCACCATCCAGAGAT |
| | | GAACTGACTAAGAACCAAGTCTCTCTGACTTGTCTGGTTAAAGGTTTC |
| | | TATCCATCTGATATTGCTGTTGAATGGGAGTCTAATGGTCAACCAGA |
| | | AAACAACTACAAGACTACTCCTCCTGTTCTGGATTCTGATGGTTCCTT |
| | | CTTCCTTTACTCTAAGCTTACTGTTGATAAGTCCAGATGGCAACAAGG |
| | | TAACGTCTTCTCATGTTCCGTTATGCATGAAGCTTTGCATAACCATTA |
| | | CACTCAGAAGTCTCTTTCCCTGTCTCCAGGTAAATAA |
| 66 | Human Fc | AEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYSKLT |
| | | VDKSRWQQGNVFSCSVMHEALHNHYTQKSLSLSPGK |
| 67 | DNA | GAGGTCCAATTGGTTGAATCTGGTGGAGGTTTGGTCCAACCAGGTGG |
| | encodes | ATCTCTGAGACTTTCTTGTGCTGCCTCTGGTTTCAACATTAAGGATAC |
| | anti-Her2 | TTACATCCACTGGGTTAGACAGGCTCCAGGTAAGGGTTTGGAGTGGG |
| | HC | TTGCTAGAATCTACCCAACCAACGGTTACACCAGATACGCTGAtTCCG |
| | | TTAAGGGTAGATTCACCATTTCCGCTGACACTTCCAAGAACACTGCTT |
| | | ACTTGCAAATGAACTCTTTGAGAGCTGAGGACACTGCCGTCTACTAC |
| | | TGTTCCAGATGGGGTGGTGACGGTTTCTACGCCATGGACTACTGGGG |
| | | TCAAGGTACCTTGGTTACTGTCTCTTCCGCTTCTACTAAGGGACCATC |
| | | CGTTTTTCCATTGGCTCCATCCTCTAAGTCTACTTCCGGTGGTACTGCT |
| | | GCTTTGGGATGTTTGGTTAAGGACTACTTCCCAGAGCCTGTTACTGTT |
| | | TCTTGGAACTCCGGTGCTTTGACTTCTGGTGTTCACACTTTCCCAGCT |
| | | GTTTTGCAATCTTCCGGTTTGTACTCCTTGTCCTCCGTTGTTACTGTTC |
| | | CATCCTCTTCCTTGGGTACTCAGACTTACATCTGTAACGTTAACCACA |
| | | AGCCATCCAACACTAAGGTTGACAAGAAGGTTGAGCCAAAGTCCTGT |
| | | GACAAGACACATACTTGTCCACCATGTCCAGCTCCAGAATTGTTGGG |
| | | TGGTCCATCCGTTTTCTTGTTCCCACCAAAGCCAAAGGACACTTTGAT |
| | | GATCTCCAGAACTCCAGAGGTTACATGTGTTGTTGTTGACGTTTCTCA |
| | | CGAGGACCCAGAGGTTAAGTTCAACTGGTACGTTGACGGTGTTGAAG |
| | | TTCACAACGCTAAGACTAAGCCAAGAGAGGAGCAGTACAACTCCACT |
| | | TACAGAGTTGTTTCCGTTTTGACTGTTTTGCACCAGGATTGGTTGAAC |
| | | GGAAAGGAGTACAAGTGTAAGGTTTCCAACAAGGCTTTGCCAGCTCC |
| | | AATCGAAAAGACTATCTCCAAGGCTAAGGGTCAACCAAGAGAGCCA |
| | | CAGGTTTACACTTTGCCACCATCCAGAGATGAGTTGACTAAGAACCA |
| | | GGTTTCCTTGACTTGTTTGGTTAAAGGATTCTACCCATCCGACATTGC |
| | | TGTTGAGTGGGAATCTAACGGTCAACCAGAGAACAACTACAAGACTA |
| | | CTCCACCAGTTTTGGATTCTGACGGTTCCTTCTTCTTGTACTCCAAGTT |
| | | GACTGTTGACAAGTCCAGATGGCAACAGGGTAACGTTTTCTCCTGTT |
| | | CCGTTATGCATGAGGCTTTGCACAACCACTACACTCAAAAGTCCTTGT |
| | | CTTTGTCCCCAGGTAAGtaa |
| 68 | Anti-Her2 | EVQLVESGGGLVQPGGSLRLSCAASGFNIKDTYIHWVRQAPGKGLEWV |
| | HC | ARIYPTNGYTRYADSVKGRFTISADTSKNTAYLQMNSLRAEDTAVYYCS |
| | | RWGGDGFYAMDYWGQGTLVTVSSASTKGPSVFPLAPSSKSTSGGTAAL |
| | | GCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPGK |
| 69 | DNA | GACATTCAGATGACaCAGTCTCCATCTTCTTTGTCCGCTTCCGTCGGT |
| | encodes | GATAGAGTTACTATCACCTGTAGAGCTTCCCAAGACGTCAACACCGC |
| | anti-Her2 | TGTCGCCTGGTACCAACAGAAGCCAGGTAAGGCTCCAAAACTTTTGA |
| | LC | TCTACTCTGCCTCTTTCTTGTACTCCGGTGTTCCATCCAGATTTTCTGG |
| | | TTCTAGATCCGGTACCGACTTCACCTTGACCATCTCTTCCTTGCAACC |
| | | AGAAGACTTCGCTACCTACTACTGTCAACAACACTACACTACTCCTC |
| | | CAACTTTCGGTCAAGGAACTAAGGTTGAGATTAAGAGAACTGTTGCT |
| | | GCTCCATCCGTTTTCATTTTCCCACCATCCGACGAACAATTGAAGTCT |
| | | GGTACAGCTTCCGTTGTTTGTTTGTTGAACAACTTCTACCCAAGAGAG |
| | | GCTAAGGTTCAGTGGAAGGTTGACAACGCTTTGCAATCCGGTAACTC |
| | | CCAAGAATCCGTTACTGAGCAGGATTCTAAGGATTCCACTTACTCCTT |
| | | GTCCTCCACTTTGACTTTGTCCAAGGCTGATTACGAGAAGCACAAGG |
| | | TTTACGCTTGTGAGGTTACACATCAGGGTTTGTCCTCCCCAGTTACTA |
| | | AGTCCTTCAACAGAGGAGAGTGTtaa |
| 70 | Anti-Her2 | DIQMTQSPSSLSASVGDRVTITCRASQDVNTAVAWYQQKPGKAPKLLIY |
| | LC | SASFLYSGVPSRFSGSRSGTDFTLTISSLQPEDFATYYCQQHYTTPPTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQ |
| | | GLSSPVTKSFNRGEC |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A Pichia pastoris host cell which has been genetically engineered to express a galactokinase activity, a UDP-galactose-4-epimerase activity, a galactose-1-phosphate uridyl transferase activity and optionally a galactose permease activity wherein the host cell is capable of using galactose as a sole carbon energy source.

2. The host cell of claim 1, wherein the host cell has been further engineered to be capable of producing recombinant glycoproteins that have hybrid or complex N-glycans that comprise galactose residues.

3. The host cell of claim 2 wherein the UDP-galactose-4-epimerase activity is provided in a fusion protein comprising the catalytic domain of a galactosyltransferase and the catalytic domain of an UDP-galactose-4-epimerase.

4. The host cell of claim 2, wherein the host cell produces glycoproteins that have complex N-glycans in which the G0:G1/G2 ratio is less than 2:1.

5. The host cell of claim 2, wherein the host cell produces glycoproteins having predominantly an N-glycan selected from the group consisting of GalGlcNAcMan5GlcNAc2; NANAGalGlcNAcMan5GlcNAc2; GalGlcNAcMan3GlcNAc2; NANAGalGlcNAcMan3GlcNAc2; GalGlcNAc2Man3GlcNAc2; Gal2GlcNAc2Man3GlcNAc2; NANAGal2GlcNAc2Man3GlcNAc2; and NANA2Gal2GlcNAc2Man3GlcNAc2.

6. The host cell of claim 2, wherein the N-glycan is a galactose-terminated N-glycan selected from the group consisting of GalGlcNAcMan5GlcNAc2; Gal2GlcNAc2Man3GlcNAc2; and Gal2GlcNAc2Man3GlcNAc2.

7. The host cell of claim 2, wherein the N-glycan is a galactose-terminated hybrid N-glycan.

8. The host cell of claim 2, wherein the N-glycan is a sialylated N-glycan selected from the group consisting of NANAGalGlcNAcMan5GlcNAc2; NANAGal2GlcNAc2Man3GlcNAc2; and NANA2Gal2GlcNAc2Man3GlcNAc2.

9. The host cell of claim 2, wherein the recombinant glycoprotein is selected from the group consisting erythropoietin (EPO); cytokines such as interferon α, interferon β, interferon γ, and interferon ω; and granulocyte-colony stimulating factor (GCSF); GM-CSF; coagulation factors such as factor VIII, factor IX, and human protein C; antithrombin III; thrombin,; soluble IgE receptor α-chain; immunoglobulins such as IgG, IgG fragments, IgG fusions, and IgM; immunoadhesions and other Fc fusion proteins such as soluble TNF receptor-Fc fusion proteins; RAGE-Fc fusion proteins; interleukins; urokinase; chymase; and urea trypsin inhibitor; IGF-binding protein; epidermal growth factor; growth hormone-releasing factor; annexin V fusion protein; angiostatin; vascular endothelial growth factor-2; myeloid progenitor inhibitory factor-1; osteoprotegerin; α-1-antitrypsin; α-feto proteins; DNase II; kringle 3 of human plasminogen; glucocerebrosidase; TNF binding protein 1; follicle stimulating hormone; cytotoxic T lymphocyte associated antigen 4-Ig; transmembrane activator and calcium modulator and cyclophilin ligand; glucagon like protein 1; and IL-2 receptor agonist.

10. A method of producing a recombinant glycoprotein in a Pichia pastoris host with N-glycans that have galactose residues, said method comprising;
a) providing a recombinant host cell that has been genetically engineered to express
(i) a glycosylation pathway that renders the host cell capable of producing recombinant glycoproteins that have hybrid or complex N-glycans that comprise galactose residues;
(ii) a galactokinase activity, a UDP-galactose-4-epimerase activity, a galactose-1-phosphate uridyl transferase activity, and optionally a galactose permease activity; and
(iii) a recombinant glycoprotein; and
b) culturing the host cells in a medium containing galactose to produce the recombinant glycoprotein that has one or more N-glycans that have galactose residues.

11. The method of claim 10 wherein the UDP-galactose-4-epimerase activity is provided in a fusion protein comprising the catalytic domain of a galactosyltransferase and the catalytic domain of an UDP-galactose-4-epimerase.

12. The method of claim 10, wherein the G0:G1/G2 ratio of the N-glycans is less than 2:1.

13. The method of claim 10, wherein the recombinant glycoprotein has predominantly an N-glycan selected from the group consisting of GalGlcNAcMan5GlcNAc2; NANAGalGlcNAcMan5GlcNAc2; GalGlcNAcMan3GlcNAc2; NANAGalGlcNAcMan3GlcNAc2; GalGlcNAc2Man3GlcNAc2; Gal2GlcNAc2Man3GlcNAc2; NANAGal2GlcNAc2Man3GlcNAc2; and NANA2Gal2GlcNAc2Man3GlcNAc2.

14. The method of claim 10, wherein the N-glycan is a galactose-terminated N-glycan selected from the group consisting of GalGlcNAcMan5GlcNAc2; Gal2GlcNAc2Man3GlcNAc2; and Gal2GlcNAc2Man3GlcNAc2.

15. The method of claim 10, wherein the N-glycan is a galactose-terminated hybrid N-glycan.

16. The method of claim 10, wherein the N-glycan is a sialylated N-glycan selected from the group consisting of NANAGalGlcNAcMan5GlcNAc2; NANAGal2GlcNAc2Man3GlcNAc2; and NANA2Gal2GlcNAc2Man3GlcNAc2.

17. The method of claim 10, wherein the recombinant glycoprotein is selected from the group consisting erythropoietin (EPO); cytokines such as interferon α, interferon β, interferon γ, and interferon ω; and granulocyte-colony stimulating factor (GCSF); GM-CSF; coagulation factors such as factor VIII, factor IX, and human protein C; antithrombin III; thrombin,; soluble IgE receptor α-chain; immunoglobulins such as IgG, IgG fragments, IgG fusions, and IgM; immunoadhesions and other Fc fusion proteins such as soluble TNF receptor-Fc fusion proteins; RAGE-Fc fusion proteins; interleukins; urokinase; chymase; and urea trypsin inhibitor; IGF-binding protein; epidermal growth factor; growth hormone-releasing factor; annexin V fusion protein; angiostatin; vascular endothelial growth factor-2; myeloid progenitor inhibitory factor-1; osteoprotegerin; α-1-antitrypsin; α-feto proteins; DNase II; kringle 3 of human plasminogen; glucocerebrosidase; TNF binding protein 1; follicle stimulating hormone; cytotoxic T lymphocyte associated antigen 4-Ig; transmembrane activator and calcium modulator and cyclophilin ligand; glucagon like protein 1; and IL-2 receptor agonist.

18. A method for producing a recombinant Pichia pastoris host cell that expresses a heterologous protein, comprising:
(a) providing a host cell that has been genetically engineered to express a one or two enzyme activities selected from the group consisting of galactokinase activity, UDP-galactose-4-epimerase activity, and galactose-1-phosphate uridyl transferase activity;
(b) transforming the host cell with one or more nucleic acid molecules encoding the heterologous protein and the enzyme or enzymes from the group in step (a) that are not expressed in the host cell of step (a); and
(c) culturing the host cells in a medium containing galactose as the sole carbon source to provide the recombinant Pichia pastoris host cell that expresses a heterologous protein.

19. The method of claim 18, wherein the host cell is further genetically engineered to express a galactose permease.

20. The method of claim 18, wherein the host cell is genetically modified to produce glycoproteins that have one or more N-glycans that comprise galactose.

21-26. (canceled)


SEQUENCE LISTING (70 sequences)

SEQ ID NO: 1 - DNA, 27 bp, Artificial Sequence
  PCR primer RCD192
gccgcgacctgagccgcctgccccaac

SEQ ID NO: 2 - DNA, 27 bp, Artificial Sequence
  PCR primer RCD186
ctagctcggtgtcccgatgtccactgt

SEQ ID NO: 3 - DNA, 36 bp, Artificial Sequence
  PCR primer RCD198
cttaggcgcgccggccgcgacctgagccgcctgccc

SEQ ID NO: 4 - DNA, 21 bp, Artificial Sequence
  PCR primer RCD201
ggggcatatctgccgcccatc

SEQ ID NO: 5 - DNA, 21 bp, Artificial Sequence
  PCR primer RCD200
gatgggcggcagatatgcccc

SEQ ID NO: 6 - DNA, 36 bp, Artificial Sequence
  PCR primer RCD199
cttcttaattaactagctcggtgtcccgatgtccac

SEQ ID NO: 7 - DNA, 49 bp, Artificial Sequence
  PCR primer DmUGT-5′
ggctcgagcggccgccaccatgaatagcatacacatgaacgccaatacg

SEQ ID NO: 8 - DNA, 47 bp, Artificial Sequence
  PCR primer DmUGT-3′
ccctcgagttaattaactagacgcgcggcagcagcttctcctcatcg

SEQ ID NO: 9 - DNA, 21 bp, Artificial Sequence
  PCR primer GALE2-L
atgactggtgttcatgaaggg

SEQ ID NO: 10 - DNA, 21 bp, Artificial Sequence
  PCR primer GALE2-R
ttacttatatgtcttggtatg

SEQ ID NO: 11 - DNA, 94 bp, Artificial Sequence
  PCR primer GD1
gcggccgcatgactggtgttcatgaagggactgtgttggttactggcggcgctggttatataggttctcatacgtgcgttgttttgttagaaaa

SEQ ID NO: 12 - DNA, 29 bp, Artificial Sequence
  PCR primer GD2
ttaattaattacttatatgtcttggtatg

SEQ ID NO: 13 - DNA, 33 bp, Artificial Sequence
  PCR primer PB158
ttagcggccgcaggaatgactaaatctcattca

SEQ ID NO: 14 - DNA, 33 bp, artficial
  PCR primer PB159
aacttaattaagcttataattcatatagacagc

SEQ ID NO: 15 - DNA, 11 bp, Artificial Sequence
  PCR primer PB156
ttagcggccgc

SEQ ID NO: 16 - DNA, 11 bp, Artificial Sequence
  PCR primer PB157
aacttaattaa

SEQ ID NO: 17 - DNA, 32 bp, Artificial Sequence
  PCR primer PB160
ttagcggccgcaggaatgactgctgaagaatt

SEQ ID NO: 18 - DNA, 34 bp, Artificial Sequence
  PCR primer PB161
aacttaattaagcttacagtctttgtagataatc

SEQ ID NO: 19 - DNA, 108 bp, Artificial Sequence
  DNA encodes Mnn2 leader (53)
atgctgcttaccaaaaggttttcaaagctgttcaagctgacgttcatagttttgatattgtgcgggctgttcgtcattacaaacaaatacatggatgagaacacgtcg

SEQ ID NO: 20 - protein, 36 aa, Artificial Sequence
  Mnn2 leader (53)
MLLTKRFSKLFKLTFIVLILCGLFVITNKYMDENTS

SEQ ID NO: 21 - DNA, 300 bp, Artificial Sequence
  DNA encodes Mnn2 leader (54)
atgctgcttaccaaaaggttttcaaagctgttcaagctgacgttcatagttttgatattgtgcgggctgttcgtcattacaaacaaatacatggatgagaacacgtcggtcaaggagtacaaggagtacttagacagatatgtccagagttactccaataagtattcatcttcctcagacgccgccagcgctgacgattcaaccccattgagggacaatgatgaggcaggcaatgaaaagttgaaaagcttctacaacaacgttttcaactttctaatggttgattcgcccgggcgcgcc

SEQ ID NO: 22 - protein, 100 aa, Artificial Sequence
  Mnn2 leader (54)
MLLTKRFSKLFKLTFIVLILCGLFVITNKYMDENTSVKEYKEYLDRYVQSYSNKYSSSSDAASADDSTPLRDNDEAGNEKLKSFYNNVFNFLMVDSPGRA

SEQ ID NO: 23 - DNA, 57 bp, Artificial Sequence
  DNA encodes S. cerevisiae Mating Factor pre signal sequence
atgagattcccatccatcttcactgctgttttgttcgctgcttcttctgctttggct

SEQ ID NO: 24 - protein, 19 aa, Artificial Sequence
  S. cerevisiae Mating Factor pre signal sequence
MRFPSIFTAVLFAASSALA

SEQ ID NO: 25 - DNA, 60 bp, Artificial Sequence
  DNA encodes alpha amylase signal sequence (from Aspergillus niger  -amylase)
atggttgcttggtggtccttgttcttgtacggattgcaagttgctgctccagctttggct

SEQ ID NO: 26 - protein, 20 aa, Artificial Sequence
  alpha amylase signal sequence (from Aspergillus niger  -amylase)
MVAWWSLFLYGLQVAAPALA

SEQ ID NO: 27 - DNA, 99 bp, Artificial Sequence
  DNA encodes Pp SEC12 (10) leader
atgcccagaaaaatatttaactacttcattttgactgtattcatggcaattcttgctattgttttacaatggtctatagagaatggacatgggcgcgcc

SEQ ID NO: 28 - protein, 33 aa, Artificial Sequence
  Pp SEC12 (10) leader
MPRKIFNYFILTVFMAILAIVLQWSIENGHGRA

SEQ ID NO: 29 - DNA, 183 bp, Artificial Sequence
  DNA encodes ScMnt1 (Kre2) (33) leader
atggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatcgggcgcgcc

SEQ ID NO: 30 - protein, 61 aa, Artificial Sequence
  ScMnt1 (Kre2) (33) leader
MALFLSKRLLRFTVIAGAVIVLLLTLNSNSRTQQYIPSSISAAFDFTSGSISPEQQVIGRA

SEQ ID NO: 31 - DNA, 318 bp, Artificial Sequence
  DNA encodes ScSEC12 (8) leader
atgaacactatccacataataaaattaccgcttaactacgccaactacacctcaatgaaacaaaaaatctctaaatttttcaccaacttcatccttattgtgctgctttcttacattttacagttctcctataagcacaatttgcattccatgcttttcaattacgcgaaggacaattttctaacgaaaagagacaccatctcttcgccctacgtagttgatgaagacttacatcaaacaactttgtttggcaaccacggtacaaaaacatctgtacctagcgtagattccataaaagtgcatggcgtggggcgcgcc

SEQ ID NO: 32 - protein, 106 aa, Artificial Sequence
  ScSEC12 (8) leader
MNTIHIIKLPLNYANYTSMKQKISKFFTNFILIVLLSYILQFSYKHNLHSMLFNYAKDNFLTKRDTISSPYVVDEDLHQTTLFGNHGTKTSVPSVDSIKVHGVGRA

SEQ ID NO: 33 - DNA, 981 bp, Mus musculus
  CDS (1)...(979): MmSLC35A3 UDP-GlcNAc transporter
atgtctgccaacctaaaatatctttccttgggaattttggtgtttcagactaccagtctggttctaacgatgcggtattctaggactttaaaagaggaggggcctcgttatctgtcttctacagcagtggttgtggctgaatttttgaagataatggcctgcatctttttagtctacaaagacagtaagtgtagtgtgagagcactgaatagagtactgcatgatgaaattcttaataagcccatggaaaccctgaagctcgctatcccgtcagggatatatactcttcagaacaacttactctatgtggcactgtcaaacctagatgcagccacttaccaggttacatatcagttgaaaatacttacaacagcattattttctgtgtctatgcttggtaaaaaattaggtgtgtaccagtggctctccctagtaattctgatggcaggagttgcttttgtacagtggccttcagattctcaagagctgaactctaaggacctttcaacaggctcacagtttgtaggcctcatggcagttctcacagcctgtttttcaagtggctttgctggagtttattttgagaaaatcttaaaagaaacaaaacagtcagtatggataaggaacattcaacttggtttctttggaagtatatttggattaatgggtgtatacgtttatgatggagaattggtctcaaagaatggattttttcagggatataatcaactgacgtggatagttgttgctctgcaggcacttggaggccttgtaatagctgctgtcatcaaatatgcagataacattttaaaaggatttgcgacctccttatccataatattgtcaacaataatatcttatttttggttgcaagattttgtgccaaccagtgtctttttccttggagccatccttgtaatagcagctactttcttgtatggttacgatcccaaacctgcaggaaatcccactaaagcatag

SEQ ID NO: 34 - protein, 326 aa, Mus musculus
MSANLKYLSLGILVFQTTSLVLTMRYSRTLKEEGPRYLSSTAVVVAEFLKIMACIFLVYKDSKCSVRALNRVLHDEILNKPMETLKLAIPSGIYTLQNNLLYVALSNLDAATYQVTYQLKILTTALFSVSMLGKKLGVYQWLSLVILMAGVAFVQWPSDSQELNSKDLSTGSQFVGLMAVLTACFSSGFAGVYFEKILKETKQSVWIRNIQLGFFGSIFGLMGVYVYDGELVSKNGFFQGYNQLTWIVVALQALGGLVIAAVIKYADNILKGFATSLSIILSTIISYFWLQDFVPTSVFFLGAILVIAATFLYGYDPKPAGNPTKA

SEQ ID NO: 35 - DNA, 1068 bp, Saccharomyces pombe
  CDS (1)...(1065): DNA encodes SpGALE
atgactggtgttcatgaagggactgtgttggttactggcggcgctggttatataggttctcatacgtgcgttgttttgttagaaaaaggatatgatgttgtaattgtcgataatttatgcaattctcgcgttgaagccgtgcaccgcattgaaaaactcactgggaaaaaagtcatattccaccaggtggatttgcttgatgagccagctttggacaaggtcttcgcaaatcaaaacatatctgctgtcattcattttgctggtctcaaagcagttggtgaatctgtacaggttcctttgagttattacaaaaataacatttccggtaccattaatttaatagagtgcatgaagaagtataatgtacgtgacttcgtcttttcttcatctgctaccgtgtatggcgatcctactagacctggtggtaccattcctattccagagtcatgccctcgtgaaggtacaagcccatatggtcgcacaaagcttttcattgaaaatatcattgaggatgagaccaaggtgaacaaatcgcttaatgcagctttattacgctattttaatcccggaggtgctcatccctctggtgaactcggtgaagatcctcttggcatccctaataacttgcttccttatatcgcgcaagttgctgtaggaagattggatcatttgaatgtatttggcgacgattatcccacatctgacggtactccaattcgtgactacattcacgtatgcgatttggcagaggctcatgttgctgctctcgattacctgcgccaacattttgttagttgccgcccttggaatttgggatcaggaactggtagtactgtttttcaggtgctcaatgcgttttcgaaagctgttggaagagatcttccttataaggtcacccctagaagagcaggggacgttgttaacctaaccgccaaccccactcgcgctaacgaggagttaaaatggaaaaccagtcgtagcatttatgaaatttgcgttgacacttggagatggcaacagaagtatccctatggctttgacctgacccataccaagacatataagtaa

SEQ ID NO: 36 - protein, 355 aa, Saccharomyces pombe
MTGVHEGTVLVTGGAGYIGSHTCVVLLEKGYDVVIVDNLCNSRVEAVHRIEKLTGKKVIFHQVDLLDEPALDKVFANQNISAVIHFAGLKAVGESVQVPLSYYKNNISGTINLIECMKKYNVRDFVFSSSATVYGDPTRPGGTIPIPESCPREGTSPYGRTKLFIENIIEDETKVNKSLNAALLRYFNPGGAHPSGELGEDPLGIPNNLLPYIAQVAVGRLDHLNVFGDDYPTSDGTPIRDYIHVCDLAEAHVAALDYLRQHFVSCRPWNLGSGTGSTVFQVLNAFSKAVGRDLPYKVTPRRAGDVVNLTANPTRANEELKWKTSRSIYEICVDTWRWQQKYPYGFDLTHTKTYK

SEQ ID NO: 37 - DNA, 1074 bp, Drosophila melangaster
  CDS (1)...(1071): DNA encodes DmUGT
atgaatagcatacacatgaacgccaatacgctgaagtacatcagcctgctgacgctgaccctgcagaatgccatcctgggcctcagcatgcgctacgcccgcacccggccaggcgacatcttcctcagctccacggccgtactcatggcagagttcgccaaactgatcacgtgcctgttcctggtcttcaacgaggagggcaaggatgcccagaagtttgtacgctcgctgcacaagaccatcattgcgaatcccatggacacgctgaaggtgtgcgtcccctcgctggtctatatcgttcaaaacaatctgctgtacgtctctgcctcccatttggatgcggccacctaccaggtgacgtaccagctgaagattctcaccacggccatgttcgcggttgtcattctgcgccgcaagctgctgaacacgcagtggggtgcgctgctgctcctggtgatgggcatcgtcctggtgcagttggcccaaacggagggtccgacgagtggctcagccggtggtgccgcagctgcagccacggccgcctcctctggcggtgctcccgagcagaacaggatgctcggactgtgggccgcactgggcgcctgcttcctctccggattcgcgggcatctactttgagaagatcctcaagggtgccgagatctccgtgtggatgcggaatgtgcagttgagtctgctcagcattcccttcggcctgctcacctgtttcgttaacgacggcagtaggatcttcgaccagggattcttcaagggctacgatctgtttgtctggtacctggtcctgctgcaggccggcggtggattgatcgttgccgtggtggtcaagtacgcggataacattctcaagggcttcgccacctcgctggccatcatcatctcgtgcgtggcctccatatacatcttcgacttcaatctcacgctgcagttcagcttcggagctggcctggtcatcgcctccatatttctctacggctacgatccggccaggtcggcgccgaagccaactatgcatggtcctggcggcgatgaggagaagctgctgccgcgcgtctag

SEQ ID NO: 38 - protein, 357 aa, Drosophila melangaster
MNSIHMNANTLKYISLLTLTLQNAILGLSMRYARTRPGDIFLSSTAVLMAEFAKLITCLFLVFNEEGKDAQKFVRSLHKTIIANPMDTLKVCVPSLVYIVQNNLLYVSASHLDAATYQVTYQLKILTTAMFAVVILRRKLLNTQWGALLLLVMGIVLVQLAQTEGPTSGSAGGAAAAATAASSGGAPEQNRMLGLWAALGACFLSGFAGIYFEKILKGAEISVWMRNVQLSLLSIPFGLLTCFVNDGSRIFDQGFFKGYDLFVWYLVLLQAGGGLIVAVVVKYADNILKGFATSLAIIISCVASIYIFDFNLTLQFSFGAGLVIASIFLYGYDPARSAPKPTMHGPGGDEEKLLPRV

SEQ ID NO: 39 - DNA, 1587 bp, Saccharomyces cerevisiae
  CDS (1)...(1584): DNA encodes ScGAL1
atgactaaatctcattcagaagaagtgattgtacctgagttcaattctagcgcaaaggaattaccaagaccattggccgaaaagtgcccgagcataattaagaaatttataagcgcttatgatgctaaaccggattttgttgctagatcgcctggtagagtcaatctaattggtgaacatattgattattgtgacttctcggttttacctttagctattgattttgatatgctttgcgccgtcaaagttttgaacgagaaaaatccatccattaccttaataaatgctgatcccaaatttgctcaaaggaagttcgatttgccgttggacggttcttatgtcacaattgatccttctgtgtcggactggtctaattactttaaatgtggtctccatgttgctcactcttttctaaagaaacttgcaccggaaaggtttgccagtgctcctctggccgggctgcaagtcttctgtgagggtgatgtaccaactggcagtggattgtcttcttcggccgcattcatttgtgccgttgctttagctgttgttaaagcgaatatgggccctggttatcatatgtccaagcaaaatttaatgcgtattacggtcgttgcagaacattatgttggtgttaacaatggcggtatggatcaggctgcctctgtttgcggtgaggaagatcatgctctatacgttgagttcaaaccgcagttgaaggctactccgtttaaatttccgcaattaaaaaaccatgaaattagctttgttattgcgaacacccttgttgtatctaacaagtttgaaaccgccccaaccaactataatttaagagtggtagaagtcactacagctgcaaatgttttagctgccacgtacggtgttgttttactttctggaaaagaaggatcgagcacgaataaaggtaatctaagagatttcatgaacgtttattatgccagatatcacaacatttccacaccctggaacggcgatattgaatccggcatcgaacggttaacaaagatgctagtactagttgaagagtctctcgccaataagaaacagggctttagtgttgacgatgtcgcacaatccttgaattgttctcgcgaagaattcacaagagactacttaacaacatctccagtgagatttcaagtcttaaagctatatcagagggctaagcatgtgtattctgaatctttaagagtcttgaaggctgtgaaattaatgactacagcgagctttactgccgacgaagactttttcaagcaatttggtgccttgatgaacgagtctcaagcttcttgcgataaactttacgaatgttcttgtccagagattgacaaaatttgttccattgctttgtcaaatggatcatatggttcccgtttgaccggagctggctggggtggttgtactgttcacttggttccagggggcccaaatggcaacatagaaaaggtaaaagaagcccttgccaatgagttctacaaggtcaagtaccctaagatcactgatgctgagctagaaaatgctatcatcgtctctaaaccagcattgggcagctgtctatatgaattataa

SEQ ID NO: 40 - protein, 528 aa, Saccharomyces cerevisiea
MTKSHSEEVIVPEFNSSAKELPRPLAEKCPSIIKKFISAYDAKPDFVARSPGRVNLIGEHIDYCDFSVLPLAIDFDMLCAVKVLNEKNPSITLINADPKFAQRKFDLPLDGSYVTIDPSVSDWSNYFKCGLHVAHSFLKKLAPERFASAPLAGLQVFCEGDVPTGSGLSSSAAFICAVALAVVKANMGPGYHMSKQNLMRITVVAEHYVGVNNGGMDQAASVCGEEDHALYVEFKPQLKATPFKFPQLKNHEISFVIANTLVVSNKFETAPTNYNLRVVEVTTAANVLAATYGVVLLSGKEGSSTNKGNLRDFMNVYYARYHNISTPWNGDIESGIERLTKMLVLVEESLANKKQGFSVDDVAQSLNCSREEFTRDYLTTSPVRFQVLKLYQRAKHVYSESLRVLKAVKLMTTASFTADEDFFKQFGALMNESQASCDKLYECSCPEIDKICSIALSNGSYGSRLTGAGWGGCTVHLVPGGPNGNIEKVKEALANEFYKVKYPKITDAELENAIIVSKPALGSCLYEL

SEQ ID NO: 41 - DNA, 1098 bp, Saccharomyces cerevisiae
  CDS (1)...(1095): DNA encodes ScGAL7
atgactgctgaagaatttgatttttctagccattcccatagacgttacaatccactaaccgattcatggatcttagtttctccacacagagctaaaagaccttggttaggtcaacaggaggctgcttacaagcccacagctcctttgtatgatccaaaatgctatctatgtcctggtaacaaaagagctactggtaacctaaacccaagatatgaatcaacgtatattttccccaatgattatgctgccgttagcgatcaacctattttaccacagaatgattccaatgaggataatcttaaaaataggctgcttaaagtgcaatctgtgagaggcaattgtttcgtcatatgttttagccccaatcataatctaaccattccacaaatgaaacaatcagatctggttcatattgttaattcttggcaagcattgactgacgatctctccagagaagcaagagaaaatcataagcctttcaaatatgtccaaatatttgaaaacaaaggtacagccatgggttgttccaacttacatccacatggccaagcttggtgcttagaatccatccctagtgaagtttcgcaagaattgaaatcttttgataaatataaacgtgaacacaatactgatttgtttgccgattacgtcaaattagaatcaagagagaagtcaagagtcgtagtggagaatgaatcctttattgttgttgttccatactgggccatctggccatttgagaccttggtcatttcaaagaagaagcttgcctcaattagccaatttaaccaaatggcgaaggaggacctcgcctcgattttaaagcaactaactattaagtatgataatttatttgaaacgagtttcccatactcaatgggtatccatcaggctcctttgaatgcgactggtgatgaattgagtaatagttggtttcacatgcatttctacccacctttactgagatcagctactgttcggaaattcttggttggttttgaattgttaggtgagcctcaaagagatttaatttcggaacaagctgctgaaaaactaagaaatttagatggtcagattcattatctacaaagactataa

SEQ ID NO: 42 - protein, 365 aa, Saccharomyces cerevisiae
MTAEEFDFSSHSHRRYNPLTDSWILVSPHRAKRPWLGQQEAAYKPTAPLYDPKCYLCPGNKRATGNLNPRYESTYIFPNDYAAVSDQPILPQNDSNEDNLKNRLLKVQSVRGNCFVICFSPNHNLTIPQMKQSDLVHIVNSWQALTDDLSREARENHKPFKYVQIFENKGTAMGCSNLHPHGQAWCLESIPSEVSQELKSFDKYKREHNTDLFADYVKLESREKSRVVVENESFIVVVPYWAIWPFETLVISKKKLASISQFNQMAKEDLASILKQLTIKYDNLFETSFPYSMGIHQAPLNATGDELSNSWFHMHFYPPLLRSATVRKFLVGFELLGEPQRDLISEQAAEKLRNLDGQIHYLQRL

SEQ ID NO: 43 - DNA, 1725 bp, Saccharomyces cerevisiae
  CDS (1)...(1722): DNA encodes ScGal permease
atggcagttgaggagaacaatgtgcctgttgtttcacagcaaccccaagctggtgaagacgtgatctcttcactcagtaaagattcccatttaagcgcacaatctcaaaagtattccaatgatgaattgaaagccggtgagtcagggcctgaaggctcccaaagtgttcctatagagatacccaagaagcccatgtctgaatatgttaccgtttccttgctttgtttgtgtgttgccttcggcggcttcatgtttggctgggataccagtactatttctgggtttgttgtccaaacagactttttgagaaggtttggtatgaaacataaggatggtacccactatttgtcaaacgtcagaacaggtttaatcgtcgccattttcaatattggctgtgcctttggtggtattatactttccaaaggtggagatatgtatggccgtaaaaagggtctttcgattgtcgtctcggtttatatagttggtattatcattcaaattgcctctatcaacaagtggtaccaatatttcattggtagaatcatatctggtttgggtgtcggcggcatcgctgtcttatgtcctatgttgatctctgaaattgctccaaagcacttgagaggcacactagtttcttgttatcagctgatgattactgcaggtatctttttgggctactgtactaattacggtacaaagagctattcgaactcagttcaatggagagttccattagggctatgtttcgcttggtcattatttatgattggcgctttgacgttagttcctgaatccccacgttatttatgtgaggtgaataaggtagaagacgccaagcgttccattgctaagtctaacaaggtgtcaccagaggatcctgccgtccaggccgagttagatctgatcatggccggtatagaagctgaaaaactggctggcaatgcgtcctggggggaattattttccaccaagaccaaagtatttcaacgtttgttgatgggtgtgtttgttcaaatgttccaacaattaaccggtaacaattattttttctactacggtaccgttattttcaagtcagttggcctggatgattcctttgaaacatccattgtcattggtgtagtcaactttgcctccactttctttagtttgtggactgtcgaaaacttggggcgtcgtaaatgtttacttttgggcgctgccactatgatggcttgtatggtcatctacgcctctgttggtgttactagattatatcctcacggtaaaagccagccatcttctaaaggtgccggtaactgtatgattgtctttacctgtttttatattttctgttatgccacaacctgggcgccagttgcctgggtcatcacagcagaatcattcccactgagagtcaagtcgaaatgtatggcgttggcctctgcttccaattgggtatgggggttcttgattgcatttttcaccccattcatcacatctgccattaacttctactacggttatgtcttcatgggctgtttggttgccatgtttttttatgtctttttctttgttccagaaactaaaggcctatcgttagaagaaattcaagaattatgggaagaaggtgttttaccttggaaatctgaaggctggattccttcatccagaagaggtaataattacgatttagaggatttacaacatgacgacaaaccgtggtacaaggccatgctagaataa

SEQ ID NO: 44 - protein, 574 aa, Saccharomyces cerevisiae
MAVEENNVPVVSQQPQAGEDVISSLSKDSHLSAQSQKYSNDELKAGESGPEGSQSVPIEIPKKPMSEYVTVSLLCLCVAFGGFMFGWDTSTISGFVVQTDFLRRFGMKHKDGTHYLSNVRTGLIVAIFNIGCAFGGIILSKGGDMYGRKKGLSIVVSVYIVGIIIQIASINKWYQYFIGRIISGLGVGGIAVLCPMLISEIAPKHLRGTLVSCYQLMITAGIFLGYCTNYGTKSYSNSVQWRVPLGLCFAWSLFMIGALTLVPESPRYLCEVNKVEDAKRSIAKSNKVSPEDPAVQAELDLIMAGIEAEKLAGNASWGELFSTKTKVFQRLLMGVFVQMFQQLTGNNYFFYYGTVIFKSVGLDDSFETSIVIGVVNFASTFFSLWTVENLGRRKCLLLGAATMMACMVIYASVGVTRLYPHGKSQPSSKGAGNCMIVFTCFYIFCYATTWAPVAWVITAESFPLRVKSKCMALASASNWVWGFLIAFFTPFITSAINFYYGYVFMGCLVAMFFYVFFFVPETKGLSLEEIQELWEEGVLPWKSEGWIPSSRRGNNYDLEDLQHDDKPWYKAMLE

SEQ ID NO: 45 - DNA, 2100 bp, Saccharomyces cerevisiae
  CDS (1)...(2097): DNA encodes ScGAL10
atgacagctcagttacaaagtgaaagtacttctaaaattgttttggttacaggtggtgctggatacattggttcacacactgtggtagagctaattgagaatggatatgactgtgttgttgctgataacctgtcgaattcaacttatgattctgtagccaggttagaggtcttgaccaagcatcacattcccttctatgaggttgatttgtgtgaccgaaaaggtctggaaaaggttttcaaagaatataaaattgattcggtaattcactttgctggtttaaaggctgtaggtgaatctacacaaatcccgctgagatactatcacaataacattttgggaactgtcgttttattagagttaatgcaacaatacaacgtttccaaatttgttttttcatcttctgctactgtctatggtgatgctacgagattcccaaatatgattcctatcccagaagaatgtcccttagggcctactaatccgtatggtcatacgaaatacgccattgagaatatcttgaatgatctttacaatagcgacaaaaaaagttggaagtttgctatcttgcgttattttaacccaattggcgcacatccctctggattaatcggagaagatccgctaggtataccaaacaatttgttgccatatatggctcaagtagctgttggtaggcgcgagaagctttacatcttcggagacgattatgattccagagatggtaccccgatcagggattatatccacgtagttgatctagcaaaaggtcatattgcagccctgcaatacctagaggcctacaatgaaaatgaaggtttgtgtcgtgagtggaacttgggttccggtaaaggttctacagtttttgaagtttatcatgcattctgcaaagcttctggtattgatcttccatacaaagttacgggcagaagagcaggtgatgttttgaacttgacggctaaaccagatagggccaaacgcgaactgaaatggcagaccgagttgcaggttgaagactcctgcaaggatttatggaaatggactactgagaatccttttggttaccagttaaggggtgtcgaggccagattttccgctgaagatatgcgttatgacgcaagatttgtgactattggtgccggcaccagatttcaagccacgtttgccaatttgggcgccagcattgttgacctgaaagtgaacggacaatcagttgttcttggctatgaaaatgaggaagggtatttgaatcctgatagtgcttatataggcgccacgatcggcaggtatgctaatcgtatttcgaagggtaagtttagtttatgcaacaaagactatcagttaaccgttaataacggcgttaatgcgaatcatagtagtatcggttctttccacagaaaaagatttttgggacccatcattcaaaatccttcaaaggatgtttttaccgccgagtacatgctgatagataatgagaaggacaccgaatttccaggtgatctattggtaaccatacagtatactgtgaacgttgcccaaaaaagtttggaaatggtatataaaggtaaattgactgctggtgaagcgacgccaataaatttaacaaatcatagttatttcaatctgaacaagccatatggagacactattgagggtacggagattatggtgcgttcaaaaaaatctgttgatgtcgacaaaaacatgattcctacgggtaatatcgtcgatagagaaattgctacctttaactctacaaagccaacggtcttaggccccaaaaatccccagtttgattgttgttttgtggtggatgaaaatgctaagccaagtcaaatcaatactctaaacaatgaattgacgcttattgtcaaggcttttcatcccgattccaatattacattagaagttttaagtacagagccaacttatcaattttataccggtgatttcttgtctgctggttacgaagcaagacaaggttttgcaattgagcctggtagatacattgatgctatcaatcaagagaactggaaagattgtgtaaccttgaaaaacggtgaaacttacgggtccaagattgtctacagattttcctga

SEQ ID NO: 46 - protein, 699 aa, Saccharomyces cerevisiae
MTAQLQSESTSKIVLVTGGAGYIGSHTVVELIENGYDCVVADNLSNSTYDSVARLEVLTKHHIPFYEVDLCDRKGLEKVFKEYKIDSVIHFAGLKAVGESTQIPLRYYHNNILGTVVLLELMQQYNVSKFVFSSSATVYGDATRFPNMIPIPEECPLGPTNPYGHTKYAIENILNDLYNSDKKSWKFAILRYFNPIGAHPSGLIGEDPLGIPNNLLPYMAQVAVGRREKLYIFGDDYDSRDGTPIRDYIHVVDLAKGHIAALQYLEAYNENEGLCREWNLGSGKGSTVFEVYHAFCKASGIDLPYKVTGRRAGDVLNLTAKPDRAKRELKWQTELQVEDSCKDLWKWTTENPFGYQLRGVEARFSAEDMRYDARFVTIGAGTRFQATFANLGASIVDLKVNGQSVVLGYENEEGYLNPDSAYIGATIGRYANRISKGKFSLCNKDYQLTVNNGVNANHSSIGSFHRKRFLGPIIQNPSKDVFTAEYMLIDNEKDTEFPGDLLVTIQYTVNVAQKSLEMVYKGKLTAGEATPINLTNHSYFNLNKPYGDTIEGTEIMVRSKKSVDVDKNMIPTGNIVDREIATFNSTKPTVLGPKNPQFDCCFVVDENAKPSQINTLNNELTLIVKAFHPDSNITLEVLSTEPTYQFYTGDFLSAGYEARQGFAIEPGRYIDAINQENWKDCVTLKNGETYGSKIVYRFS

SEQ ID NO: 47 - DNA, 1047 bp, Homo sapiens
  CDS (1)...(1044): DNA encodes human GalE
atggcagagaaggtgctggtaacaggtggggctggctacattggcagccacacggtgctggagctgctggaggctggctacttgcctgtggtcatcgataacttccataatgccttccgtggagggggctccctgcctgagagcctgcggcgggtccaggagctgacaggccgctctgtggagtttgaggagatggacattttggaccagggagccctacagcgtctcttcaaaaagtacagctttatggcggtcatccactttgcggggctcaaggccgtgggcgagtcggtgcagaagcctctggattattacagagttaacctgaccgggaccatccagcttctggagatcatgaaggcccacggggtgaagaacctggtgttcagcagctcagccactgtgtacgggaacccccagtacctgccccttgatgaggcccaccccacgggtggttgtaccaacccttacggcaagtccaagttcttcatcgaggaaatgatccgggacctgtgccaggcagacaagacttggaacgcagtgctgctgcgctatttcaaccccacaggtgcccatgcctctggctgcattggtgaggatccccagggcatacccaacaacctcatgccttatgtctcccaggtggcgatcgggcgacgggaggccctgaatgtctttggcaatgactatgacacagaggatggcacaggtgtccgggattacatccatgtcgtggatctggccaagggccacattgcagccttaaggaagctgaaagaacagtgtggctgccggatctacaacctgggcacgggcacaggctattcagtgctgcagatggtccaggctatggagaaggcctctgggaagaagatcccgtacaaggtggtggcacggcgggaaggtgatgtggcagcctgttacgccaaccccagcctggcccaagaggagctggggtggacagcagccttagggctggacaggatgtgtgaggatctctggcgctggcagaagcagaatccttcaggctttggcacgcaagcctga

SEQ ID NO: 48 - protein, 348 aa, Homo sapiens
MAEKVLVTGGAGYIGSHTVLELLEAGYLPVVIDNFHNAFRGGGSLPESLRRVQELTGRSVEFEEMDILDQGALQRLFKKYSFMAVIHFAGLKAVGESVQKPLDYYRVNLTGTIQLLEIMKAHGVKNLVFSSSATVYGNPQYLPLDEAHPTGGCTNPYGKSKFFIEEMIRDLCQADKTWNAVLLRYFNPTGAHASGCIGEDPQGIPNNLMPYVSQVAIGRREALNVFGNDYDTEDGTGVRDYIHVVDLAKGHIAALRKLKEQCGCRIYNLGTGTGYSVLQMVQAMEKASGKKIPYKVVARREGDVAACYANPSLAQEELGWTAALGLDRMCEDLWRWQKQNPSGFGTQA

SEQ ID NO: 49 - DNA, 1065 bp, Artificial Sequence
  DNA encodes hGalT I catalytic domain
ggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagc

SEQ ID NO: 50 - protein, 355 aa, Artificial Sequence
  hGalT I catalytic domain
GRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 51 - DNA, 1224 bp, Artificial Sequence
  DNA encodes hGnT I catalytic domain codon-optimized
tcagtcagtgctcttgatggtgacccagcaagtttgaccagagaagtgattagattggcccaagacgcagaggtggagttggagagacaacgtggactgctgcagcaaatcggagatgcattgtctagtcaaagaggtagggtgcctaccgcagctcctccagcacagcctagagtgcatgtgacccctgcaccagctgtgattcctatcttggtcatcgcctgtgacagatctactgttagaagatgtctggacaagctgttgcattacagaccatctgctgagttgttccctatcatcgttagtcaagactgtggtcacgaggagactgcccaagccatcgcctcctacggatctgctgtcactcacatcagacagcctgacctgtcatctattgctgtgccaccagaccacagaaagttccaaggttactacaagatcgctagacactacagatgggcattgggtcaagtcttcagacagtttagattccctgctgctgtggtggtggaggatgacttggaggtggctcctgacttctttgagtactttagagcaacctatccattgctgaaggcagacccatccctgtggtgtgtctctgcctggaatgacaacggtaaggagcaaatggtggacgcttctaggcctgagctgttgtacagaaccgacttctttcctggtctgggatggttgctgttggctgagttgtgggctgagttggagcctaagtggccaaaggcattctgggacgactggatgagaagacctgagcaaagacagggtagagcctgtatcagacctgagatctcaagaaccatgacctttggtagaaagggagtgtctcacggtcaattctttgaccaacacttgaagtttatcaagctgaaccagcaatttgtgcacttcacccaactggacctgtcttacttgcagagagaggcctatgacagagatttcctagctagagtctacggagctcctcaactgcaagtggagaaagtgaggaccaatgacagaaaggagttgggagaggtgagagtgcagtacactggtagggactcctttaaggctttcgctaaggctctgggtgtcatggatgaccttaagtctggagttcctagagctggttacagaggtattgtcacctttcaattcagaggtagaagagtccacttggctcctccacctacttgggagggttatgatccttcttggaattag

SEQ ID NO: 52 - protein, 407 aa, Artificial Sequence
  Human GnT I catalytic doman
SVSALDGDPASLTREVIRLAQDAEVELERQRGLLQQIGDALSSQRGRVPTAAPPAQPRVHVTPAPAVIPILVIACDRSTVRRCLDKLLHYRPSAELFPIIVSQDCGHEETAQAIASYGSAVTHIRQPDLSSIAVPPDHRKFQGYYKIARHYRWALGQVFRQFRFPAAVVVEDDLEVAPDFFEYFRATYPLLKADPSLWCVSAWNDNGKEQMVDASRPELLYRTDFFPGLGWLLLAELWAELEPKWPKAFWDDWMRRPEQRQGRACIRPEISRTMTFGRKGVSHGQFFDQHLKFIKLNQQFVHFTQLDLSYLQREAYDRDFLARVYGAPQLQVEKVRTNDRKELGEVRVQYTGRDSFKAFAKALGVMDDLKSGVPRAGYRGIVTFQFRGRRVHLAPPPTWEGYDPSWN

SEQ ID NO: 53 - DNA, 1407 bp, Artificial Sequence
  DNA encodes Mm ManI catalytic domain
gagcccgctgacgccaccatccgtgagaagagggcaaagatcaaagagatgatgacccatgcttggaataattataaacgctatgcgtggggcttgaacgaactgaaacctatatcaaaagaaggccattcaagcagtttgtttggcaacatcaaaggagctacaatagtagatgccctggatacccttttcattatgggcatgaagactgaatttcaagaagctaaatcgtggattaaaaaatatttagattttaatgtgaatgctgaagtttctgtttttgaagtcaacatacgcttcgtcggtggactgctgtcagcctactatttgtccggagaggagatatttcgaaagaaagcagtggaacttggggtaaaattgctacctgcatttcatactccctctggaataccttgggcattgctgaatatgaaaagtgggatcgggcggaactggccctgggcctctggaggcagcagtatcctggccgaatttggaactctgcatttagagtttatgcacttgtcccacttatcaggagacccagtctttgccgaaaaggttatgaaaattcgaacagtgttgaacaaactggacaaaccagaaggcctttatcctaactatctgaaccccagtagtggacagtggggtcaacatcatgtgtcggttggaggacttggagacagcttttatgaatatttgcttaaggcgtggttaatgtctgacaagacagatctcgaagccaagaagatgtattttgatgctgttcaggccatcgagactcacttgatccgcaagtcaagtgggggactaacgtacatcgcagagtggaaggggggcctcctggaacacaagatgggccacctgacgtgctttgcaggaggcatgtttgcacttggggcagatggagctccggaagcccgggcccaacactaccttgaactcggagctgaaattgcccgcacttgtcatgaatcttataatcgtacatatgtgaagttgggaccggaagcgtttcgatttgatggcggtgtggaagctattgccacgaggcaaaatgaaaagtattacatcttacggcccgaggtcatcgagacatacatgtacatgtggcgactgactcacgaccccaagtacaggacctgggcctgggaagccgtggaggctctagaaagtcactgcagagtgaacggaggctactcaggcttacgggatgtttacattgcccgtgagagttatgacgatgtccagcaaagtttcttcctggcagagacactgaagtatttgtacttgatattttccgatgatgaccttcttccactagaacactggatcttcaacaccgaggctcatcctttccctatactccgtgaacagaagaaggaaattgatggcaaagagaaatga

SEQ ID NO: 54 - protein, 468 aa, Artificial Sequence
  Mm ManI catalytic doman
EPADATIREKRAKIKEMMTHAWNNYKRYAWGLNELKPISKEGHSSSLFGNIKGATIVDALDTLFIMGMKTEFQEAKSWIKKYLDFNVNAEVSVFEVNIRFVGGLLSAYYLSGEEIFRKKAVELGVKLLPAFHTPSGIPWALLNMKSGIGRNWPWASGGSSILAEFGTLHLEFMHLSHLSGDPVFAEKVMKIRTVLNKLDKPEGLYPNYLNPSSGQWGQHHVSVGGLGDSFYEYLLKAWLMSDKTDLEAKKMYFDAVQAIETHLIRKSSGGLTYIAEWKGGLLEHKMGHLTCFAGGMFALGADGAPEARAQHYLELGAEIARTCHESYNRTYVKLGPEAFRFDGGVEAIATRQNEKYYILRPEVIETYMYMWRLTHDPKYRTWAWEAVEALESHCRVNGGYSGLRDVYIARESYDDVQQSFFLAETLKYLYLIFSDDDLLPLEHWIFNTEAHPFPILREQKKEIDGKEK

SEQ ID NO: 55 - DNA, 1494 bp, Artificial Sequence
  DNA encodes Tr ManI catalytic domain
cgcgccggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgcttaa

SEQ ID NO: 56 - protein, 497 aa, Artificial Sequence
  Tr Man I catalytic doman
RAGSPNPTRAAAVKAAFQTSWNAYHHFAFPHDDLHPVSNSFDDERNGWGSSAIDGLDTAILMGDADIVNTILQYVPQINFTTTAVANQGISVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLAADSTIAHLASHPSTRKDLTFLSSYNGQSTSPNSGHLASFAGGNFILGGILLNEQKYIDFGIKLASSYFATYNQTASGIGPEGFAWVDSVTGAGGSPPSSQSGFYSSAGFWVTAPYYILRPETLESLYYAYRVTGDSKWQDLAWEAFSAIEDACRAGSAYSSINDVTQANGGGASDDMESFWFAEALKYAYLIFAEESDVQVQANGGNKFVFNTEAHPFSIRSSSRRGGHLA

SEQ ID NO: 57 - DNA, 1068 bp, Artificial Sequence
  DNA encodes rat GnTII catalytic domain (TC)
tccctagtgtaccagttgaactttgatcagatgctgaggaatgtcgataaagacggcacctggagtccgggggagctggtgctggtggtccaagtgcataacaggccggaatacctcaggctgctgatagactcgcttcgaaaagcccagggtattcgcgaagtcctagtcatctttagccatgacttctggtcggcagagatcaacagtctgatctctagtgtggacttctgtccggttctgcaagtgttctttccgttcagcattcagctgtacccgagtgagtttccgggtagtgatcccagagattgccccagagacctgaagaagaatgcagctctcaagttggggtgcatcaatgccgaatacccagactccttcggccattacagagaggccaaattctcgcaaaccaaacatcactggtggtggaagctgcattttgtatgggaaagagtcaaagttcttcaagattacactggccttatacttttcctggaagaggaccactacttagccccagacttttaccatgtcttcaaaaagatgtggaaattgaagcagcaggagtgtcctgggtgtgacgtcctctctctagggacctacaccaccattcggagtttctatggtattgctgacaaagtagatgtgaaaacttggaaatcgacagagcacaatatggggctagccttgacccgagatgcatatcagaagcttatcgagtgcacggacactttctgtacttacgatgattataactgggactggactcttcaatatttgactctagcttgtcttcctaaagtctggaaagtcttagttcctcaagctcctaggatttttcatgctggagactgtggtatgcatcacaagaaaacatgtaggccatccacccagagtgcccaaattgagtcattattaaataataataaacagtacctgtttccagaaactctagttatcggtgagaagtttcctatggcagccatttccccacctaggaaaaatggagggtggggagatattagggaccatgaactctgtaaaagttatagaagactgcagtga

SEQ ID NO: 58 - protein, 355 aa, Artificial Sequence
  Rat GnTII (TC)
SLVYQLNFDQMLRNVDKDGTWSPGELVLVVQVHNRPEYLRLLIDSLRKAQGIREVLVIFSHDFWSAEINSLISSVDFCPVLQVFFPFSIQLYPSEFPGSDPRDCPRDLKKNAALKLGCINAEYPDSFGHYREAKFSQTKHHWWWKLHFVWERVKVLQDYTGLILFLEEDHYLAPDFYHVFKKMWKLKQQECPGCDVLSLGTYTTIRSFYGIADKVDVKTWKSTEHNMGLALTRDAYQKLIECTDTFCTYDDYNWDWTLQYLTLACLPKVWKVLVPQAPRIFHAGDCGMHHKKTCRPSTQSAQIESLLNNNKQYLFPETLVIGEKFPMAAISPPRKNGGWGDIRDHELCKSYRRLQ

SEQ ID NO: 59 - DNA, 1068 bp, Artificial Sequence
  DNA encodes rat GnTII catalytic domain (TC) codon-optimized
tccttggtttaccaattgaacttcgaccagatgttgagaaacgttgacaaggacggtacttggtctcctggtgagttggttttggttgttcaggttcacaacagaccagagtacttgagattgttgatcgactccttgagaaaggctcaaggtatcagagaggttttggttatcttctcccacgatttctggtctgctgagatcaactccttgatctcctccgttgacttctgtccagttttgcaggttttcttcccattctccatccaattgtacccatctgagttcccaggttctgatccaagagactgtccaagagacttgaagaagaacgctgctttgaagttgggttgtatcaacgctgaatacccagattctttcggtcactacagagaggctaagttctcccaaactaagcatcattggtggtggaagttgcactttgtttgggagagagttaaggttttgcaggactacactggattgatcttgttcttggaggaggatcattacttggctccagacttctaccacgttttcaagaagatgtggaagttgaagcaacaagagtgtccaggttgtgacgttttgtccttgggaacttacactactatcagatccttctacggtatcgctgacaaggttgacgttaagacttggaagtccactgaacacaacatgggattggctttgactagagatgcttaccagaagttgatcgagtgtactgacactttctgtacttacgacgactacaactgggactggactttgcagtacttgactttggcttgtttgccaaaagtttggaaggttttggttccacaggctccaagaattttccacgctggtgactgtggaatgcaccacaagaaaacttgtagaccatccactcagtccgctcaaattgagtccttgttgaacaacaacaagcagtacttgttcccagagactttggttatcggagagaagtttccaatggctgctatttccccaccaagaaagaatggtggatggggtgatattagagaccacgagttgtgtaaatcctacagaagattgcagtag

SEQ ID NO: 60 - DNA, 1240 bp, Artificial Sequence
  DNA encodes rat GnTII catalytic domain (TA)
aggaagaacgacgcccttgccccgccgctgctggactcggagcccctacggggtgcgggccatttcgccgcgtccgtaggcatccgcagggtttctaacgactcggccgctcctctggttcccgcggtcccgcggccggaggtggacaacctaacgctgcggtaccggtccctagtgtaccagttgaactttgatcagatgctgaggaatgtcgataaagacggcacctggagtccgggggagctggtgctggtggtccaagtgcataacaggccggaatacctcaggctgctgatagactcgcttcgaaaagcccagggtattcgcgaagtcctagtcatctttagccatgacttctggtcggcagagatcaacagtctgatctctagtgtggacttctgtccggttctgcaagtgttctttccgttcagcattcagctgtacccgagtgagtttccgggtagtgatcccagagattgccccagagacctgaagaagaatgcagctctcaagttggggtgcatcaatgccgaatacccagactccttcggccattacagagaggccaaattctcgcaaaccaaacatcactggtggtggaagctgcattttgtatgggaaagagtcaaagttcttcaagattacactggccttatacttttcctggaagaggaccactacttagccccagacttttaccatgtcttcaaaaagatgtggaaattgaagcagcaggagtgtcctgggtgtgacgtcctctctctagggacctacaccaccattcggagtttctatggtattgctgacaaagtagatgtgaaaacttggaaatcgacagagcacaatatggggctagccttgacccgagatgcatatcagaagcttatcgagtgcacggacactttctgtacttacgatgattataactgggactggactcttcaatatttgactctagcttgtcttcctaaagtctggaaagtcttagttcctcaagctcctaggatttttcatgctggagactgtggtatgcatcacaagaaaacatgtaggccatccacccagagtgcccaaattgagtcattattaaataataataaacagtacctgtttccagaaactctagttatcggtgagaagtttcctatggcagccatttccccacctaggaaaaatggagggtggggagatattagggaccatgaactctgtaaaagttatagaagactgcagtgagtta

SEQ ID NO: 61 - protein, 411 aa, Artificial Sequence
  rat GnTII catalytic domain (TA)
RKNDALAPPLLDSEPLRGAGHFAASVGIRRVSNDSAAPLVPAVPRPEVDNLTLRYRSLVYQLNFDQMLRNVDKDGTWSPGELVLVVQVHNRPEYLRLLIDSLRKAQGIREVLVIFSHDFWSAEINSLISSVDFCPVLQVFFPFSIQLYPSEFPGSDPRDCPRDLKKNAALKLGCINAEYPDSFGHYREAKFSQTKHHWWWKLHFVWERVKVLQDYTGLILFLEEDHYLAPDFYHVFKKMWKLKQQECPGCDVLSLGTYTTIRSFYGIADKVDVKTWKSTEHNMGLALTRDAYQKLIECTDTFCTYDDYNWDWTLQYLTLACLPKVWKVLVPQAPRIFHAGDCGMHHKKTCRPSTQSAQIESLLNNNKQYLFPETLVIGEKFPMAAISPPRKNGGWGDIRDHELCKSYRRLQ

SEQ ID NO: 62 - DNA, 3105 bp, Artificial Sequence
  DNA encodes Dm ManII catalytic domain (KD)
cgcgacgatccaataagacctccacttaaagtggctcgttccccgaggccagggcaatgccaagatgtggtccaagacgtgcccaatgtggatgtacagatgctggagctatacgatcgcatgtccttcaaggacatagatggaggcgtgtggaaacagggctggaacattaagtacgatccactgaagtacaacgcccatcacaaactaaaagtcttcgttgtgccgcactcgcacaacgatcctggatggattcagacgtttgaggaatactaccagcacgacaccaagcacatcctgtccaatgcactacggcatctgcacgacaatcccgagatgaagttcatctgggcggaaatctcctactttgctcggttctatcacgatttgggagagaacaaaaagctgcagatgaagtccattgtaaagaatggacagttggaatttgtgactggaggatgggtaatgccggacgaggccaactcccactggcgaaacgtactgctgcagctgaccgaagggcaaacatggttgaagcaattcatgaatgtcacacccactgcttcctgggccatcgatcccttcggacacagtcccactatgccgtacattttgcagaagagtggtttcaagaatatgcttatccaaaggacgcactattcggttaagaaggaactggcccaacagcgacagcttgagttcctgtggcgccagatctgggacaacaaaggggacacagctctcttcacccacatgatgcccttctactcgtacgacattcctcatacctgtggtccagatcccaaggtttgctgtcagttcgatttcaaacgaatgggctccttcggtttgagttgtccatggaaggtgccgccgcgtacaatcagtgatcaaaatgtggcagcacgctcagatctgctggttgatcagtggaagaagaaggccgagctgtatcgcacaaacgtgctgctgattccgttgggtgacgacttccgcttcaagcagaacaccgagtgggatgtgcagcgcgtgaactacgaaaggctgttcgaacacatcaacagccaggcccacttcaatgtccaggcgcagttcggcacactgcaggaatactttgatgcagtgcaccaggcggaaagggcgggacaagccgagtttcccacgctaagcggtgactttttcacatacgccgatcgatcggataactattggagtggctactacacatcccgcccgtatcataagcgcatggaccgcgtcctgatgcactatgtacgtgcagcagaaatgctttccgcctggcactcctgggacggtatggcccgcatcgaggaacgtctggagcaggcccgcagggagctgtcattgttccagcaccacgacggtataactggcacagcaaaaacgcacgtagtcgtcgactacgagcaacgcatgcaggaagctttaaaagcctgtcaaatggtaatgcaacagtcggtctaccgattgctgacaaagccctccatctacagtccggacttcagtttctcgtactttacgctcgacgactcccgctggccaggatctggtgtggaggacagtcgaaccaccataatactgggcgaggatatactgccctccaagcatgtggtgatgcacaacaccctgccccactggcgggagcagctggtggacttttatgtatccagtccgtttgtaagcgttaccgacttggcaaacaatccggtggaggctcaggtgtccccggtgtggagctggcaccacgacacactcacaaagactatccacccacaaggctccaccaccaagtaccgcatcatcttcaaggctcgggtgccgcccatgggcttggccacctacgttttaaccatctccgattccaagccagagcacacctcgtatgcatcgaatctcttgctccgtaaaaacccgacttcgttaccattgggccaatatccggaggatgtgaagtttggcgatcctcgagagatctcattgcgggttggtaacggacccaccttggccttttcggagcagggtctccttaagtccattcagcttactcaggatagcccacatgtaccggtgcacttcaagttcctcaagtatggcgttcgatcgcatggcgatagatccggtgcctatctgttcctgcccaatggaccagcttcgccagtcgagcttggccagccagtggtcctggtgactaagggcaaactggagtcgtccgtgagcgtgggacttccgagcgtggtgcaccagacgataatgcgcggtggtgcacctgagattcgcaatctggtggatataggctcactggacaacacggagatcgtgatgcgcttggagacgcatatcgacagcggcgatatcttctacacggatctcaatggattgcaatttatcaagaggcggcgtttggacaaattacctttgcaggccaactattatcccataccttctggtatgttcattgaggatgccaatacgcgactcactctcctcacgggtcaaccgctgggtggatcttctctggcctcgggcgagctagagattatgcaagatcgtcgcctggccagcgatgatgaacgcggcctgggacagggtgttttggacaacaagccggtgctgcatatttatcggctggtgctggagaaggttaacaactgtgtccgaccgtcaaagcttcatcctgccggctatttgacaagtgccgcacacaaagcatcgcagtcactgctggatccactggacaagtttatattcgctgaaaatgagtggatcggggcacaggggcaatttggtggcgatcatccttcggctcgtgaggatctcgatgtgtcggtgatgagacgcttaaccaagagctcggccaaaacccagcgagtaggctacgttctgcaccgcaccaatctgatgcaatgcggcactccagaggagcatacacagaagctggatgtgtgccacctactgccgaatgtggcgagatgcgagcgcacgacgctgactttcctgcagaatttggagcacttggatggcatggtggcgccggaagtgtgccccatggaaaccgccgcttatgtgagcagtcactcaagctga

SEQ ID NO: 63 - protein, 1034 aa, Artificial Sequence
  Dm ManII catalytic doman (KD)
RDDPIRPPLKVARSPRPGQCQDVVQDVPNVDVQMLELYDRMSFKDIDGGVWKQGWNIKYDPLKYNAHHKLKVFVVPHSHNDPGWIQTFEEYYQHDTKHILSNALRHLHDNPEMKFIWAEISYFARFYHDLGENKKLQMKSIVKNGQLEFVTGGWVMPDEANSHWRNVLLQLTEGQTWLKQFMNVTPTASWAIDPFGHSPTMPYILQKSGFKNMLIQRTHYSVKKELAQQRQLEFLWRQIWDNKGDTALFTHMMPFYSYDIPHTCGPDPKVCCQFDFKRMGSFGLSCPWKVPPRTISDQNVAARSDLLVDQWKKKAELYRTNVLLIPLGDDFRFKQNTEWDVQRVNYERLFEHINSQAHFNVQAQFGTLQEYFDAVHQAERAGQAEFPTLSGDFFTYADRSDNYWSGYYTSRPYHKRMDRVLMHYVRAAEMLSAWHSWDGMARIEERLEQARRELSLFQHHDGITGTAKTHVVVDYEQRMQEALKACQMVMQQSVYRLLTKPSIYSPDFSFSYFTLDDSRWPGSGVEDSRTTIILGEDILPSKHVVMHNTLPHWREQLVDFYVSSPFVSVTDLANNPVEAQVSPVWSWHHDTLTKTIHPQGSTTKYRIIFKARVPPMGLATYVLTISDSKPEHTSYASNLLLRKNPTSLPLGQYPEDVKFGDPREISLRVGNGPTLAFSEQGLLKSIQLTQDSPHVPVHFKFLKYGVRSHGDRSGAYLFLPNGPASPVELGQPVVLVTKGKLESSVSVGLPSVVHQTIMRGGAPEIRNLVDIGSLDNTEIVMRLETHIDSGDIFYTDLNGLQFIKRRRLDKLPLQANYYPIPSGMFIEDANTRLTLLTGQPLGGSSLASGELEIMQDRRLASDDERGLGQGVLDNKPVLHIYRLVLEKVNNCVRPSKLHPAGYLTSAAHKASQSLLDPLDKFIFAENEWIGAQGQFGGDHPSAREDLDVSVMRRLTKSSAKTQRVGYVLHRTNLMQCGTPEEHTQKLDVCHLLPNVARCERTTLTFLQNLEHLDGMVAPEVCPMETAAYVSSHSS

SEQ ID NO: 64 - DNA, 3105 bp, Artificial Sequence
  DNA encodes Dm ManII catalytic domain (KD) codon-optimized
agagacgatccaattagacctccattgaaggttgctagatccccaagaccaggtcaatgtcaagatgttgttcaggacgtcccaaacgttgatgtccagatgttggagttgtacgatagaatgtccttcaaggacattgatggtggtgtttggaagcagggttggaacattaagtacgatccattgaagtacaacgctcatcacaagttgaaggtcttcgttgtcccacactcccacaacgatcctggttggattcagaccttcgaggaatactaccagcacgacaccaagcacatcttgtccaacgctttgagacatttgcacgacaacccagagatgaagttcatctgggctgaaatctcctacttcgctagattctaccacgatttgggtgagaacaagaagttgcagatgaagtccatcgtcaagaacggtcagttggaattcgtcactggtggatgggtcatgccagacgaggctaactcccactggagaaacgttttgttgcagttgaccgaaggtcaaacttggttgaagcaattcatgaacgtcactccaactgcttcctgggctatcgatccattcggacactctccaactatgccatacattttgcagaagtctggtttcaagaatatgttgatccagagaacccactactccgttaagaaggagttggctcaacagagacagttggagttcttgtggagacagatctgggacaacaaaggtgacactgctttgttcacccacatgatgccattctactcttacgacattcctcatacctgtggtccagatccaaaggtttgttgtcagttcgatttcaaaagaatgggttccttcggtttgtcttgtccatggaaggttccacctagaactatctctgatcaaaatgttgctgctagatccgatttgttggttgatcagtggaagaagaaggctgagttgtacagaaccaacgtcttgttgattccattgggtgacgacttcagattcaagcagaacaccgagtgggatgttcagagagtcaactacgaaagattgttcgaacacatcaactctcaggctcacttcaatgtccaggctcagttcggtactttgcaggaatacttcgatgctgttcaccaggctgaaagagctggacaagctgagttcccaaccttgtctggtgacttcttcacttacgctgatagatctgataactactggtctggttactacacttccagaccataccataagagaatggacagagtcttgatgcactacgttagagctgctgaaatgttgtccgcttggcactcctgggacggtatggctagaatcgaggaaagattggagcaggctagaagagagttgtccttgttccagcaccacgacggtattactggtactgctaaaactcacgttgtcgtcgactacgagcaaagaatgcaggaagctttgaaagcttgtcaaatggtcatgcaacagtctgtctacagattgttgactaagccatccatctactctccagacttctccttctcctacttcactttggacgactccagatggccaggttctggtgttgaggactctagaactaccatcatcttgggtgaggatatcttgccatccaagcatgttgtcatgcacaacaccttgccacactggagagagcagttggttgacttctacgtctcctctccattcgtttctgttaccgacttggctaacaatccagttgaggctcaggtttctccagtttggtcttggcaccacgacactttgactaagactatccacccacaaggttccaccaccaagtacagaatcatcttcaaggctagagttccaccaatgggtttggctacctacgttttgaccatctccgattccaagccagagcacacctcctacgcttccaatttgttgcttagaaagaacccaacttccttgccattgggtcaatacccagaggatgtcaagttcggtgatccaagagagatctccttgagagttggtaacggtccaaccttggctttctctgagcagggtttgttgaagtccattcagttgactcaggattctccacatgttccagttcacttcaagttcttgaagtacggtgttagatctcatggtgatagatctggtgcttacttgttcttgccaaatggtccagcttctccagtcgagttgggtcagccagttgtcttggtcactaagggtaaattggagtcttccgtttctgttggtttgccatctgtcgttcaccagaccatcatgagaggtggtgctccagagattagaaatttggtcgatattggttctttggacaacactgagatcgtcatgagattggagactcatatcgactctggtgatatcttctacactgatttgaatggattgcaattcatcaagaggagaagattggacaagttgccattgcaggctaactactacccaattccatctggtatgttcattgaggatgctaataccagattgactttgttgaccggtcaaccattgggtggatcttctttggcttctggtgagttggagattatgcaagatagaagattggcttctgatgatgaaagaggtttgggtcagggtgttttggacaacaagccagttttgcatatttacagattggtcttggagaaggttaacaactgtgtcagaccatctaagttgcatccagctggttacttgacttctgctgctcacaaagcttctcagtctttgttggatccattggacaagttcatcttcgctgaaaatgagtggatcggtgctcagggtcaattcggtggtgatcatccatctgctagagaggatttggatgtctctgtcatgagaagattgaccaagtcttctgctaaaacccagagagttggttacgttttgcacagaaccaatttgatgcaatgtggtactccagaggagcatactcagaagttggatgtctgtcacttgttgccaaatgttgctagatgtgagagaactaccttgactttcttgcagaatttggagcacttggatggtatggttgctccagaagtttgtccaatggaaaccgctgcttacgtctcttctcactcttcttga

SEQ ID NO: 65 - DNA, 702 bp, Artificial Sequence
  DNA encodes human Fc
gctgaaccaaaatcttgtgataaaactcatacatgtccaccatgtccagctcctgaacttctgggtggaccatcagttttcttgttcccaccaaaaccaaaggatacccttatgatttctagaactcctgaagtcacatgtgttgttgttgatgtttctcatgaagatcctgaagtcaagttcaactggtacgttgatggtgttgaagttcataatgctaagacaaagccaagagaagaacaatacaactctacttacagagttgtctctgttcttactgttctgcatcaagattggctgaatggtaaggaatacaagtgtaaggtctccaacaaagctcttccagctccaattgagaaaaccatttccaaagctaaaggtcaaccaagagaaccacaagtttacaccttgccaccatccagagatgaactgactaagaaccaagtctctctgacttgtctggttaaaggtttctatccatctgatattgctgttgaatgggagtctaatggtcaaccagaaaacaactacaagactactcctcctgttctggattctgatggttccttcttcctttactctaagcttactgttgataagtccagatggcaacaaggtaacgtcttctcatgttccgttatgcatgaagctttgcataaccattacactcagaagtctctttccctgtctccaggtaaataa

SEQ ID NO: 66 - protein, 233 aa, Artificial Sequence
  Human Fc
AEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 1353 bp, Artificial Sequence
  DNA encodes anti-Her2 HC
gaggtccaattggttgaatctggtggaggtttggtccaaccaggtggatctctgagactttcttgtgctgcctctggtttcaacattaaggatacttacatccactgggttagacaggctccaggtaagggtttggagtgggttgctagaatctacccaaccaacggttacaccagatacgctgattccgttaagggtagattcaccatttccgctgacacttccaagaacactgcttacttgcaaatgaactctttgagagctgaggacactgccgtctactactgttccagatggggtggtgacggtttctacgccatggactactggggtcaaggtaccttggttactgtctcttccgcttctactaagggaccatccgtttttccattggctccatcctctaagtctacttccggtggtactgctgctttgggatgtttggttaaggactacttcccagagcctgttactgtttcttggaactccggtgctttgacttctggtgttcacactttcccagctgttttgcaatcttccggtttgtactccttgtcctccgttgttactgttccatcctcttccttgggtactcagacttacatctgtaacgttaaccacaagccatccaacactaaggttgacaagaaggttgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaaaggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgcacaaccactacactcaaaagtccttgtctttgtccccaggtaagtaa

SEQ ID NO: 68 - protein, 450 aa, Artificial Sequence
  Anti-Her2 HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 645 bp, Artificial Sequence
  DNA encodes anti-Her2 LC
gacattcagatgacacagtctccatcttctttgtccgcttccgtcggtgatagagttactatcacctgtagagcttcccaagacgtcaacaccgctgtcgcctggtaccaacagaagccaggtaaggctccaaaacttttgatctactctgcctctttcttgtactccggtgttccatccagattttctggttctagatccggtaccgacttcaccttgaccatctcttccttgcaaccagaagacttcgctacctactactgtcaacaacactacactactcctccaactttcggtcaaggaactaaggttgagattaagagaactgttgctgctccatccgttttcattttcccaccatccgacgaacaattgaagtctggtacagcttccgttgtttgtttgttgaacaacttctacccaagagaggctaaggttcagtggaaggttgacaacgctttgcaatccggtaactcccaagaatccgttactgagcaggattctaaggattccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 70 - protein, 214 aa, Artificial Sequence
  Anti-Her2 LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC